(19) United States
(12) Patent Application Publication
(10) Pub. No.: US 2026/0226192 A1
(43) Pub. Date: Aug. 6, 2026

(54) AGENTS, METHODS AND USES THEREOF

(71) Applicant: CREASALLIS LTD, Cambridge (GB)

(72) Inventor: Zahra JAWAD, Cambridge (GB)

(21) Appl. No.: 19/152,082

(22) PCT Filed: Feb. 14, 2024
(86) PCT No.: PCT/GB2024/050397

§ 371 Date: Jul. 30, 2025

(30) Foreign Application Priority Data
Feb. 14, 2023 (GB) 2302074.6

(51) Int. Cl. C07K 16/32; A61K 39/00; C07K 16/22; C07K 16/28; C07K 16/30

(52) U.S. Cl. CPC C07K 16/32; C07K 16/22; C07K 16/2818; C07K 16/2827; C07K 16/2863; C07K 16/3007; A61K 2039/505; C07K 2317/31; C07K 2317/53; C07K 2317/54; C07K 2317/569; C07K 2317/622; C07K 2317/92; C07K 2317/94; C07K 2319/50

(57) ABSTRACT

The present invention relates to methods and conjugates to improve the penetrability of biological substances into the tumour microenvironment (TME) for therapeutic purposes, suitably the agent comprises at least: i. a therapeutic domain; ii. a cleavable domain; and iii. a stabilisation domain.

### FIELD OF THE INVENTION

The present invention relates to methods and conjugates to improve the penetrability of biological substances into the tumour microenvironment (TME) for therapeutic purposes.

### BACKGROUND OF THE INVENTION

Cancers are a major contributor to disease burden worldwide, and projections forecast that global cancer burden will continue to grow. Cancer is a generic term for a large group of diseases that can affect any part of the body. A defining feature of cancer is the rapid creation of unwanted cells that grow beyond their usual boundaries, and which can then invade adjoining parts of the body and spread to other organs.

Antibody-based therapies have revolutionised therapies, particularly in the field of oncology. Engineered antigen-binding domains can bind to specific tumour markers and can activate or inactivate signalling pathways. This results in the slowing down or stopping of cell proliferation and tumour growth, or the shrinkage of tumours (Zahavi D, Weiner L (2020). Monoclonal Antibodies in Cancer Therapy. Antibodies (Basel). 9(3):34). Engineered antigen-binding domains are also used in immunotherapy, where they can activate or deactivate immune cells within the tumour to stop or slow down tumour proliferation and growth.

The success of these antibody-based therapies is due to the high specificity of the antigen-binding domains to their oncological targets. This mitigates any non-specific binding to similar antigens giving rise to better targeting. Antibody-based therapies can be multivalent, so a single antibody can engage its target multiple times which gives rise to additional efficacy that small molecules do not give (Imai K, Takaoka A (2006). Comparing antibody and small-molecule therapies for cancer. Nat Rev Cancer. 6(9):714-27).

Antibodies also contain a fragment crystallisable (Fc) region. This region has several functions, one of which is to extend the antibody's half-life. This is in part achieved by the Fc region increasing the size of the antibody taking it past the glomerular filtration barrier (GFB) threshold.

A main problem in the field remains the penetration of the antibody-based therapies into the TME. It is estimated that only 0.001-7% of the injected antibody makes it into the heart of the tumour (Khongorzul et al. (2020). Antibody-Drug Conjugates: A Comprehensive Review. Mol Cancer Res. 18(1):3-19). A lower penetration of a biologic into the TME is associated with a sub-optimal efficacy of the biologic. This sub-optimal efficacy leads to the use of higher doses of the biologic being administered to achieve a therapeutic effect; however, higher doses can be detrimental and/or toxic to the patient.

A contributing factor of the lower penetration is thought to be the large sizes of antibodies and thus, much effort has been focused on generating small antibody fragments for better efficacy. Modelling experiments also suggest that smaller antibody fragments have better penetration into tissues (Thurber et al. (2008). Antibody tumour penetration: transport opposed by systemic and antigen-mediated clearance. Adv Drug Deliv Rev. 60(12):1421-34).

The small antibody fragments often a lack of an Fc region, which lowers the half-life (Li et al., 2019) of the antibody and results in a lower efficacy.

In heathy vascularised tissue, cells are only a few micrometres (μm) away from a blood vessel, so organ/cell penetration is not of concern. However, the large cell mass acquired in tumour formation causes an increase in the distance between the nearest blood vessel and the cells. This makes accessibility and delivery of therapies to the cells an issue.

This also gives marked characteristics within the tumour that are unusual. For example, the distance from blood vessels lead to hypoxia within the tumour; low concentrations of oxygen, elevated levels of carbon dioxide resulting in lower pH levels. In addition, the environment promotes carcinogenesis. To achieve this, specific proteases are expressed which allow for rogue cells to escape into the blood stream, invade local tissues and eventually metastasise. Thus, during tumour development, aberrantly expressed proteases are found in the TME. The expression of different protease combinations varies depending on the tumour type and location. Such proteases have been used to specifically target antibody activity in vivo for examples WO2013192546A1 and WO2015066279A2.

Some protease cleavage sites are well known in the scientific literature, and cleavable domains comprising such cleavage sites can be readily constructed using established genetic engineering techniques and/or by chemical synthesis techniques known in the art, while other protease cleavage sites are not well known so designing such cleavable domain requires more experimentation.

Although these have been used to improve specificity of antibodies, the problem remains that the antibodies have poor penetration and thus poor efficacy. The present invention seeks to solve this problem.

### SUMMARY OF THE INVENTION

The invention is based on the use of tumour-specific proteases to enhance antibody penetration by specific cleavage of the antibody at specific engineered sites when the antibody has reached the TME. The invention is based on the discovery that such sequences around the hinge region of antibodies does not affect the expression or function of the antibody.

The invention is also based on cleavage of the therapeutic agent and thus reduction of its size at the tumour or in the vicinity of the unwanted cells or the tumour significantly improving specificity, penetrability and therefore efficacy of the therapeutic agent.

Accordingly, in one aspect, the invention provides an engineered agent comprising at least: i. a therapeutic domain; ii. a cleavable domain; and iii. a stabilisation domain wherein the cleavable domain is positioned between the therapeutic domain and the stabilising domain.

The invention provides introduction of a cleavable domain between a therapeutic domain and a stabilisation domain, which cleavage site is selectively cleaved in the vicinity of the unwanted cells, the therapeutic domain can be released from a stabilisation domain in the vicinity of the unwanted cells and can bind to an antigen on or in the unwanted cells.

The invention provides means to improve the penetrability of an agent into a tumour by using size and diffusion to penetrate deeper into the hypoxic parts of the tumour to achieve enhanced agent efficacy.

### BRIEF DESCRIPTION OF THE FIGURES

FIG. 1—Illustrative schematic of where the therapeutic domain is Fab and the cleavage site is within the upper or lower the hinge region of an antibody. Upon entering the TME, conditions allow for the cleavage of the antibody into 3 domains, the Fab domains (x2) and Fc domain (x1).

FIG. 2—Illustrative schematic of where the therapeutic domain is Fab and the cleavable domain is present after the hinge region of an antibody. Upon entering the TME, conditions allow for the cleavage of the antibody into 2 domains, the F(ab′)2 domains (x1) and Fc domain (x1).

FIG. 3—Illustrative schematic of where the therapeutic domain is not Fab and the cleavable domain is present before the hinge region of an Fc containing entity. Upon entering the TME, conditions allow for the cleavage of the agent into 3 domains, the therapeutic domains (x2) and Fc domain (x1).

FIG. 4—Illustrative schematic of where the therapeutic domain is not Fab and the cleavable domain is present after the hinge region of an Fc containing entity. Upon entering the TME, conditions allow for the cleavage of the agent into 2 domains, the therapeutic domain (x1) and Fc domain (x1).

FIG. 5—Example of where the cleavable domain is present at a linker domain that separates the therapeutic domain from the stabilisation domain of a biologic.

FIG. 6—Illustrative schematic of different positions of the cleavable domain within the hinge region. (A) D1-D4 designs—the cleavable site is within the upper hinge region; (B) D6-D7 designs—the cleavable site is within the lower hinge region; (C) D5 design was engineered with a glycosylation site and gives a negative control. The position and length of the linkers differ from design to design. The cleavable site may also be within the middle hinge (not shown).

FIG. 7—Assessment of antibody concentration by Protein A Octet.

FIG. 8—Gel image showing Protein Quality Analysis (SDS-PAGE).

FIG. 9—Repeat assessment of antibody concentration by Protein A Octet.

FIG. 10—Repeat gel image showing Protein Quality Analysis (SDS-PAGE). The box shows the antibody heavy chains (~50 kDa).

FIG. 11—Chromatographic assessment of candidate expression and purification. (A) trastuzumab (WT also known as mAb) Control and (B) D1.

FIG. 12—Gel image showing (A) Non-Reduced Protein Quality Analysis (SDS-PAGE) and (B) Reduced Protein Quality Analysis (SDS-PAGE). Lanes: 1=Molecular Weight Marker in kilodaltons (KDa); 2=Trastuzumab mAb; 3=Trastuzumab D1; 4=Trastuzumab D2; 5=Trastuzumab D3; 6=Trastuzumab D4; 7=Trastuzumab D5; 8=Trastuzumab D6; 9=Trastuzumab D7.

FIG. 13—Binding of trastuzumab mAb and engineered designs to hHER2.

FIG. 14—12-point dilution kinetics measurement. (A) trastuzumab mAb and (B) D1.

FIG. 15—Gel image showing liberation of F(ab) for D1-D5 and liberation of F(ab′)2 for D6-D7 fragments from control mAb, D1 and D2 antibodies by uPA Protease. 1=Molecular Weight Marker; 2=mAb+0 nM Protease; 3=mAb+23.5 nM Protease; 4=mAb+235 nM Protease; 5=D1+0 nM Protease; 6=D1+23.5 nM Protease; 7=D1+235 nM Protease; 8=D2 0 nM Protease; 9=D2+23.5 nM Protease; 10=D2+235 nM Protease; 11=D3+0 nM Protease; 12=D3+23.5 nM Protease; 13=mAb+235 nM Protease; 14=D4+0 nM Protease; 15=D4+23.5 nM Protease; 16=D4+235 nM Protease; 17=D5+0 nM Protease; 18=D5+23.5 nM Protease; 19=D5+235 nM Protease; 20=D6+0 nM Protease; 21=D6+23.5 nM Protease; 22=D6+235 nM Protease; 23=D7+0 nM Protease; 24=D7+23.5 nM Protease; 25=D7+235 nM Protease. Fc is the middle row of bands, and F(ab) and F(ab′)2+/Light chain is the lower row of bands. F(ab) and F(ab′)2 appear as the same size on the gel because DTT reduces the liberated F(ab)′2 into 2 Fabs.

FIG. 16—Binding of trastuzumab and engineered antibodies to HER2 after uPA treatment—Fab detection.

FIG. 17—Binding of Trastuzumab and engineered antibodies to FC Gamma Receptors. (A) FC Gamma Receptor Ia; (B) FC Gamma Receptor IIa; (C) FC Gamma Receptor IIb; (D) FC Gamma Receptor IIIa.

FIG. 18—Larger Scale 20 mg Expression of (A) Trastuzumab mAb 98.5% monomer and (B) Trastuzumab D3 94.3% monomer.

FIG. 19—SDS PAGE gel showing expression and purity of Trastuzumab-mAb and D3. 1=Trastuzumab-mAb and 2=Trastuzumab D3. NR=non-reduced and R=reduced. A PageRuler Plus prestained ladder was used.

FIG. 20—Binding of Larger Scale Preps of trastuzumab mAb and D3 to hHER2 by ELISA.

FIG. 21—Expression of Trastuzumab-mAb and Trastuzumab D1 in CHO-K1 Cell Lines.

FIG. 22—Comparison of expressions from CHO-K1 and Expi-CHO.

FIG. 23—SDS-PAGE gel showing expression and purity of trastuzumab engineered antibodies with different cleavage sites 1. Molecular Weight Marker, 2. Empty Lane, 3. Trastuzumab D9 (with ADAM10 site)

FIG. 24—Binding of Trastuzumab mAb and Trastuzumab D3 and D9 to hHER2.

FIG. 25—Non reduced SDS-PAGE gel showing the expression and purity of 1. Molecular Weight Marker, 2. Bevacizumab mAb, 3. Bevacizumab D13, 4. Cetuximab mAb, 5. Cetuximab D12, 6. M5A mAb, 7. M5A D15, 8. Pembrolizumab mAb, 9. Pembrolizumab D14a, 10. Pembrolizumab D14b, 11., Ipilimumab mAb, 12. Ipilimumab D16.

FIG. 26—(A) Binding of Cetuximab mAb and Cetuximab D12 to hEGFR; (B) Binding of Bevacizumab mAb and Bevacizumab D13 to hVEGF.

FIG. 27—SDS-PAGE gel showing protease digestion of (A) 1. Molecular Weight Marker, 2. Bevacizumab mAb+0 nM Protease, 3. Bevacizumab mAb+23.5 nM Protease, 4. Bevacizumab mAb+235 nM Protease, 5. Bevacizumab D13+0 nM Protease, 6. Bevacizumab D13+23.5 nM Protease, 7. Bevacizumab D13+235 nM Protease, 8. Cetuximab mAb+0 nM Protease, 9. Cetuximab mAb+23.5 nM Protease, 10. Cetuximab mAb+235 nM Protease, 11. Cetuximab D12+0 nM Protease, 12. Cetuximab D12+23.5 nM Protease, 13. Cetuximab D12+235 nM Protease; (B) 1. Molecular Weight Marker, 2. M5A mAb+0 nM Protease, 3. M5A mAb+23.5 nM Protease, 4. M5A mAb+235 nM Protease, 5. M5A D15+0 nM Protease, 6. M5A D15+23.5 nM Protease, 7. M5A D15+235 nM Protease (C) 1. Molecular Weight Marker, 2. Pembrolizumab mAb, 3. Pembrolizumab D14a+500 μM Protease, 4. Pembrolizumab D14b+500 μM; (D) 1. Molecular Weight Marker, 2. Trastuzumab D3 3. Trastuzumab D9. Protease was added at a 1:1 molar concentration of antibody to protease.

FIG. 28—Binding of different antibody-derived fragments to their respective targets (A) Pembrolizumab, D14a and D14b to hPD1-HIS; (B) M5A and D15 to hCEACAM-5; (C) Ipililmumab and D16 to hCTLA-4. Insertion of the cleavage side does not significantly affect the ability of the engineered antibody to bind to its target.

FIG. 29—Illustrative schematic of different types of experimentally-tested therapeutic domains. (A) An agent wherein the therapeutic domain is Fab; (B) An agent wherein the therapeutic domain is VHH; (C) An agent wherein the therapeutic domain is ScFv. In (A), (B) and (C) the cleavable domain is located in the upper hinge and the stabilisation domain is an Fc region.

FIG. 30—SDS PAGE gel showing (A) expression and purity of various engineered agents comprising VHH or ScFv as a therapeutic domain. Lanes: 1=Molecular Weight Marker; 2=anti-EGFR VHH-FC (D17); 3=anti-EGFR VHH Control (7D12-FC); 4=anti-Her2 ScFv-FC (D18); 5=anti-Her2 ScFv-FC control (Trastuzumab VI-Vh long linker); (B) protease digestion of VHH and ScFv-FC constructs with uPA. Lanes: 1. Molecular Weight Marker, Lane 2. D17 with 250 μM uPA; 3=anti-EGFR VHH Control with 250 μM uPA; 4=D18 with 250 μM uPA; 5=anti-Her2 ScFv-FC control with 250 μM uPA. All proteases were added with a 1:1 molar concentration of antibody to protease; (C) Assessment of binding of anti-EGFR VHH-FC (D17) to hEGFR by ELISA; (D) Assessment of binding of anti-Her2 ScFv-FC (D18) to hHER2 by ELISA.

FIG. 31—Binding of tumour samples to human Her2 with Vk light chain detection (A) In a mouse study, 5 “Group 2” (G2) mice were injected with Trastuzumab mAb and 5 “Group 3” (G3) mice were injected with Trastuzumab D3. G3 mice showed an increased accumulation of Trastuzumab D3 in the tumour in comparison to G2 mice's accumulation of Trastuzumab mAb. (B) Amalgamation of data from (A) mouse study showing that there 15.6 μM Trastuzumab D3 in G3 mice and 7.8 μM Trastuzumab mAb in G2.

### DETAILED DESCRIPTION OF THE INVENTION

The agent may be a therapeutic agent. By a “therapeutic agent”, we include the meaning of it having a beneficial or desired result including and preferably a beneficial or desired clinical result, i.e. a treatment (curative) agent or a “prophylactic” agent administered to treat and/or prevent disease. By agent, we include the meaning of biological agent which include proteins, oligopeptides, polypeptides, enzymes, antibodies and parts thereof, vaccines, nucleotides and the like, antibody analogues, antibody mimetics, immunoglobulins, immunomodulators, blood, blood components, cells, allergens, genes, viruses, toxins, venoms or combinations thereof.

In one embodiment, the agent is an engineered agent. By “engineered agent” we include the meaning that the agent is not a naturally occurring agent and may be synthesised. In one embodiment, the stabilisation domain and therapeutic domain are naturally occurring together (such as in an antibody); whereas the cleavable domain is engineered into the hinge region.

By ‘therapeutic domain’, we include the meaning of any domain or entity which has therapeutically beneficial effect on a subject. The therapeutic domain may bind to an endogenous target, for example may bind a tumour antigen that is endogenous to the subject.

By ‘cleavable domain’, we include the meaning of any cleavable domain or entity which is susceptible to cleavage at the tumour by another domain or entity. Cleavage can occur at one or more locations within the cleavable domain. Such a site within the cleavable domain is known as cleavage site. Preferably, the cleavable domain is a peptide that includes a substrate for an enzyme.

In antibodies, there are naturally occurring cleavage sites for non-tumour specific proteases and or bacterial enzymes (Brezski and Jordan. 2010. Cleavage of IgGs by proteases associated with invasive diseases. mAbs 2:3, 212-220). In one embodiment the agent of the invention does not include a naturally occurring cleavage site. Accordingly, in one embodiment the agent of the invention does not comprise any cleavable domains that are susceptible to following enzymes: papain, pepsin, glutamyl endopeptidase I (GluV8), immunoglobulin-degrading enzyme of Streptococcus pyogenes (IdeS), Streptopain (SpeB), pseudolysin, mirabilysin, trepolisin.

In one embodiment, the cleavable domain is artificially introduced into a naturally occurring agent. By “the cleavable domain is artificially introduced” we include the meaning that the cleavable domain is not found in the naturally occurring agent. The agent is engineered such that a cleavable domain is artificially introduced.

As used herein, the term “antibody” includes but is not limited to polyclonal, monoclonal, chimeric, single chain, Fab fragments, fragments produced by a Fab expression library and multispecific (e.g. bispecific) antibodies. Such fragments include fragments of whole antibodies which retain their binding activity for a target substance, Fv, F(ab′) and F(ab′)2 fragments, as well as single chain antibodies (scFv), fusion proteins and other synthetic proteins which comprise the antigen-binding site of the antibody. A targeting moiety comprising only part of an antibody may be advantageous by virtue of optimising the rate of clearance from the blood and may be less likely to undergo non-specific binding due to the Fc region. Also included are domain antibodies (dAbs), diabodies, nanobodies (such as camelid antibodies, engineered camelid antibodies, shark antibodies or llama antibodies). The advantages of using antibody fragments, rather than whole antibodies, are several-fold. The smaller size of the fragments may lead to improved pharmacological properties, such as better penetration of solid tissue. Moreover, antigen-binding fragments such as Fab, Fv, ScFv and dAb antibody fragments can be expressed in and secreted from E. coli or yeast, thus allowing convenient production in the laboratory and economical production on a commercial scale.

The antibody may be of any of the IgG, IgE, IgA, IgM and IgD classes and may be derived from any species. If the antibody is an IgG, it may be any of IgG1, IgG2, IgG3 or IgG4. It is preferred, however, that when the agent is for administration to a particular host, that the antibody, or at least the constant regions thereof, are derived from that host. The antibodies may be human antibodies in the sense that they have the amino acid sequence of human antibodies with specificity for the selected antigen. Alternatively, they may be mouse, chimeric or humanized antibodies. For example, when the agent is to be administered to a human, the antibody is preferably a human antibody or a humanized antibody, and so on.

Suitable antibodies that bind to particular antigens expressed by unwanted cells can be made by the skilled person using technology long-established in the art. Methods of preparation of monoclonal antibodies and antibody fragments are well known in the art and include hybridoma technology.

By ‘stabilisation domain’, we include the meaning of any domain or entity that is able to stabilise or extend the half-life of the agent in a biological system by reducing or inhibiting degradation and/or reducing or inhibiting clearance of the whole or part(s) of the agent.

In an embodiment, the agent comprises amino acids. Preferably, the agent is a protein, peptide, bicyclic peptide, tricyclic peptide or a polypeptide. The term “protein” as used herein takes its conventional meaning, namely a plurality of amino acids that are linked together via a peptide bond, to form a polypeptide polymer chain. In an additional embodiment, the agent comprises non-natural isomers or amino acids.

Polynucleotides which encode suitable therapeutic domains are known in the art or can be readily designed from known sequences such as from sequences of proteins known to interact with surface markers expressed on unwanted cells or contained in nucleotide sequence databases such as the GenBank, EMBL and dbEST databases. Polynucleotides which encode suitable stabilising domains are known in the art or can readily be designed from known sequences and made. Polynucleotides which encode suitable cleavable domains are known in the art or can readily be designed from known sequences and made. Those skilled in the art would be capable of making such agents, which are typically established based on known approaches, such as chemical synthesis techniques and/or genetic engineering techniques.

In genetic engineering techniques, the nucleic acid is expressed in a suitable host to produce an engineered agent of the invention. Thus, the nucleic acid encoding the agent of the invention may be used in accordance with known techniques, appropriately modified in view of the teachings contained herein, to construct an expression vector, which is then used to transform an appropriate host cell for the expression and production of the agent of the invention of the invention.

It is appreciated that the nucleic acid encoding the agent of the invention may be joined to a wide variety of other nucleic acid sequences for introduction into an appropriate host. The companion nucleic acid will depend upon the nature of the host, the manner of the introduction of the nucleic acid into the host, and whether episomal maintenance or integration is desired, as is well known in the art.

Amino acid residues described herein are generally in the natural “L” isomeric form. However, residues in the “D” isomeric form can be substituted for L-amino acid residues in certain situations, provided that the agent of the invention still retains its function. The definition also includes, unless otherwise specifically indicated, chemically modified amino acids, including amino acid analogues (such as penicillamine, 3-mercapto-D-valine), naturally occurring non-proteogenic amino acids (such as norleucine), beta-amino acids, azapeptides, N-methylated amino acids and chemically synthesised compounds that have properties known in the art to be characteristic of an amino acid. The term “proteogenic” indicates that the amino acid can be incorporated into a protein in a cell through well-known metabolic pathways. The definition also includes amino acids in which the functional side group has been chemically derivatised. Such derivatised molecules include, for example, those molecules in which free amino groups have been derivatised to form amine hydrochlorides, p-toluene sulfonyl groups, carbobenzoxy groups, t-butyloxycarbonyl groups, chloroacetyl groups or formyl groups. Free carboxyl groups may be derivatised to form salts, methyl and ethyl esters or other types of esters or hydrazides. Free hydroxyl groups may be derivatised to form O-acyl or O-alkyl derivatives. Also included as derivatives are those peptide portions that contain one or more naturally occurring amino acid derivatives of the twenty standard amino acids.

It is appreciated that the peptide portions of the agent of the invention can be peptide “mimetics”, i.e. peptidomimetics which mimic the structural features of peptides comprising or consisting of the amino acid sequence as described herein. Peptidomimetics can be even more advantageous in therapeutic use, in the resistance to degradation, in permeability or in possible oral administration.

Non protein entities (such as PEG molecules) can be fused using well known methods in the art. Covalent chemical conjugation techniques may include but are not limited to: (i) adding a C-terminal cysteine residue, and conjugating through this via maleimide chemistry, (ii) conjugation through lysines; (iii) using His tags to conjugate.

As is well known, the glomerular filtration barrier (GFB) is a highly specialised blood filtration interface that displays a high conductance to small and midsized solutes in plasma but retains relative impermeability to macromolecules. Therefore, the barrier enables the renal elimination of small and midsized solutes but stops renal elimination of macromolecules, functioning as a sieve that typically only lets water and small solutes pass through to be cleared by the kidneys. In one embodiment, the agent is a size which prevents it from penetrating the glomerular filtration barrier. Suitable agents above a certain size will be unable to pass through the GFB and therefore will not be eliminated by the kidney. It will be appreciated by those skilled in the art, that the GFB threshold size will vary from one species of animal to the next and may even vary from subject to subject. Moreover, in disease, there can be a change in glomerular permselectivity, thereby making the GFB “leakier” such that macromolecules (e.g. albumin) can be eliminated by the kidney. Other properties of the agent may also have an effect on preventing the agent from penetrating the GFB, for example, the charge, composition, or surface modifications.

In some embodiments, the agent has a size of at least 6 nanometres (nm), at least 6.5 nm, at least 7 nm, at least 7.5 nm, at least 8 nm, at least 8.5 nm, at least 9 nm, at least 9.5 nm, at least 10 nm. By “size”, we include the meaning of the hydrodynamic diameter of the agent. Those skilled in the art would be capable of selecting an appropriate assay to measure the size of the agent. For example, the hydrodynamic diameter may be measured using Dynamic Light Scattering (DLS) or any standard technique in the art.

In another embodiment, the agent has a molecular weight of at least 40 kilodaltons (kDa), at least 41 kDa, at least 42 kDa, at least 43 kDa, at least 44 kDa, at least 45 kDa, at least 46 kDa, at least 47 kDa, at least 48 kDa, at least 49 kDa, at least 50 kDa, at least 51 kDa, at least 52 kDa, at least 53 kDa, at least 54 kDa, at least 55 kDa, at least 56 kDa, at least 57 kDa, at least 58 kDa, at least 59 kDa, at least 60 kDa, at least 61 kDa, at least 62 kDa, at least 63 kDa, at least 64 kDa, at least 65 kDa, at least 66 kDa, at least 67 kDa, at least 68 kDa, at least 69 kDa, at least 70 kDa, at least 71 kDa, at least 72 kDa, at least 73 kDa, at least 74 kDa, at least 75 kDa, at least 76 kDa, at least 77 kDa, at least 78 kDa, at least 79 kDa, at least 80 kDa, at least 90 kDa, at least 100 kDa, at least 150 kDa.

The molecular weight of an agent substance, also called the molar mass, M, is the mass of 1 mole of that substance, given in M gram. The molecular weight of an agent can be measured or calculated by standard techniques known in the art, such as mass spectrometry, methods based on viscosity and light-scattering or sodium dodecyl sulfate-polyacrylamide gel electrophoresis (SDS-PAGE).

In some embodiments, the half-life of the agent in a biological system is at least 10 hours, at least 12 hours, at least 14 hours, at least 16 hours, at least 18 hours, at least 20 hours, at least 22 hours, at least 24 hours, at least 26 hours, at least 28 hours, at least 30 hours, at least 32 hours, at least 36 hours, at least 38 hours, at least 40 hours, at least 42 hours, at least 44 hours, at least 46 hours, at least 48 hours, at least one week, at least two weeks, at least three weeks, at least four weeks, at least a month, at least two months.

In some embodiments, the half-life in a biological system is measured between about 35° C. and about 40° C., between about 36° C. and about 39° C., between about 36.5° C. and about 37.5° C. “Half-life” (“t_{1/2}”, “pharmacokinetic (PK)”) is well-known in the art to mean the time taken for the amount of the active agent in the body to decrease by 50%. Those skilled in the art would be capable of selecting an appropriate assay (for example, an enzyme-linked immunosorbent assay (ELISA)) to measure the amount of the agent in the serum at regular intervals over time.

An agent with a longer half-life will take longer to eliminate from the biological system and thus increase the amount of agent that is exposed to the TME. In some embodiments, the half-life in a biological system of the agent is at least 1.2, 1.4, 1.6, 1.8, 2, 2.2, 2.4, 2.6, 2.8, 3, 4, 5, 6, 7, 8, 9, 10, 11, 12, 13, 14, or at least 15 times longer, or at least 48, 50, 100, 200, 250, 265, 275, 300 times longer than the half-life of the therapeutic domain when present in a biological system in isolation from (i.e., when not conjugated to) the cleavable domain and/or the stabilisation domain.

### Therapeutic Domain

In some embodiments, the therapeutic domain is a domain that, when present in isolation from the cleavable domain and the stabilisation domain, in a biological system, has a half-life of less than 10 hours, less than 9.5 hours, less than 9 hours, less than 8.5 hours, less than 8 hours, less than 7.5 hours, less than 7 hours, less than 6.5 hours, less than 6 hours, less than 5.5 hours, less than 5 hours, less than 4.5 hours, less than 4 hours, less than 3.5 hours, less than 3 hours, less than 2.5 hours, less than 2 hours, less than 1.5 hours, less than 1 hour, less than 60 minutes, less than 40 minutes, less than 20 minutes, less than 10 minutes, less than 5 minutes, less than 2 minutes.

By “penetrate a solid tumour”, we include the meaning that the agent or therapeutic domains are taken up by a solid tumour e.g., by diffusion, intracellular transport (e.g. transcytosis), or paracellular transport. Preferably, the agent or therapeutic domains are taken up by a solid tumour by diffusion. Those skilled in the art would be capable of selecting an appropriate method to measure the ability of the agent to penetrate a solid tumour, for example by immunostaining or immunofluorescence of the agent in an in vivo tumour, organoid or tumoroid analysis, a xenograft mouse model or an in an in vitro tumour model.

By “solid tumour”, we include the meaning of heterotypic aggregates of different cell types, including for example, cancer cells, cancer stem cells, connective-tissue cells, and immune cells. In an embodiment, the solid tumour is malignant. In some embodiments, the solid tumour can be a carcinoma, a sarcoma, a lymphoma, or a melanoma.

In some embodiments, the ability of the therapeutic domain to penetrate a solid tumour when present in isolation from (i.e., when not conjugated to) the cleavable domain and/or the stabilisation domain, is increased relative to the ability of the agent.

In some embodiments, the increase is at least 1.5-fold, at least 2-fold, at least 2.5-fold, at least 2-fold, at least 2.5-fold, at least 3-fold, at least 3.5-fold, at least 4-fold, at least 4.5-fold, at least a 5-fold increase, at least 6-fold, at least 7-fold, at least 8-fold, at least 9-fold, at least a 10-fold increase, at least a 15-fold increase, at least a 20-fold increase, at least 25-fold, at least 30-fold, or at least 35-fold.

‘Tumour uptake level’ is the amount of administered therapeutic agent that is actually taken up by the tumour. In some embodiments, the tumour uptake level of the therapeutic domain when cleaved from the agent is at least 8% ID/g, preferably at least 9% ID/g, more preferably at 10% ID/g. By “% ID/g”, we include the meaning of average concentration of total antibody (bound+free) in the tumour (Schmidt and Wittrup, 2009). Those skilled in the art would be capable of selecting an appropriate method to measure the tumour uptake level, for example by immunostaining, immunofluorescence or radioactive labelling of the agent in an in vivo tumour, a xenograft mouse model or an in an in vitro tumour model.

In some embodiments, the increased penetrability and/or tumour uptake levels are sustained. In another embodiment, the increased penetrability and/or tumour uptake levels are transient. By “sustained”, we include the meaning that the increased levels of the agent are maintained over time and/or continue to penetrate the solid tumour. By “maintained over time”, we include the meaning of the increased levels being maintained for a time of at least 30 minutes, at least 40 minutes, at least 50 minutes, at least 60 minutes, at least 1 hour, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 24 hours, at least 48 hours, at least 36 hours. By “transient”, we include the meaning that the increased levels of the agent are not maintained and/or decrease over time, for example after 20 minutes or less, 15 minutes or less, 10 minutes or less, 5 minutes or less, 4 minutes or less, 3 minutes or less, 2 minutes or less, 1 minute or less, 0.5 minutes or less.

In some embodiments, after cleavage of the therapeutic domain from the agent, the tumour uptake level of the therapeutic domain is increased relative to the tumour uptake level of the agent. In some embodiments, the increase is at least at least 5-fold, at least 6-fold, at least 7-fold, at least 8-fold, at least 9-fold, at least 10-fold, at least 15-fold, at least a 20-fold, at least 25-fold, at least 30-fold, or at least 35-fold.

Preferably, the therapeutic domain is selected from any one or more of: i. an antigen-binding domain; ii. a Fab region; iii. a F(ab′)2 region; iv. a scFv region; v. a tandem scFv region; vi. a domain antibody, preferably a single domain antibody (sdAb); vii. a nanobody; viii. a monoclonal antibody; ix. a polyclonal antibody; x. a diabody; xi. a triabody; xii. A tetrabody; xiii. A pentabody; xiv. A hexabody; xv. an antibody drug conjugate; xvi. a bispecific peptide, such as a bispecific antibody; xvii. a multispecific peptide, such as a multispecific antibody; xx. a bicyclic peptide; xxi. a tricyclic peptide. Most preferably, the therapeutic domain is any one of i. an antigen-binding domain; ii. a Fab region; iii. a F(ab′)2 region; iv. a scFv region; v. a tandem scFv region. In one embodiment, the therapeutic domain is not a single domain antibody (sdAb).

Preferably, the therapeutic domain is or is derived from a Fab region, a F(ab′)2 region or a scFv region.

In one embodiment, the therapeutic domain comprises no more than one binding domain. In a further embodiment, the therapeutic domain does act as a cross-linker between the tumour and T cells.

In some embodiments, the therapeutic region further comprises a hinge region.

By “antigen-binding domains”, we include the meaning of domains that can bind to an antigen. Such domains include antibody parts and non-antibody parts. By “antibody parts thereof”, we include the meaning of an antibody fragments (such as Fab, Fv, ScFv, dAb, nanobodies). By “non-antibody parts”, we include the meaning of non-antibody type scaffolds such as affibodies, affilins, anticalins, atrimers, DARPins, FN3 scaffolds (such as adnectins and centyrins), fynomers, kunitz domains, pronectins, obodies, inhibitor cystine knots (also known as cys-knots or knottins), thyrodoxin repeats, fibronectin domains, lipocalin.

Fragment antigen-binding (Fab) regions are regions of an antibody that bind to antigens. A Fab region is composed of one constant domain and one variable domain of each of the heavy chain and the light chain (FIG. 1). Fab regions have a monovalent epitope binding site.

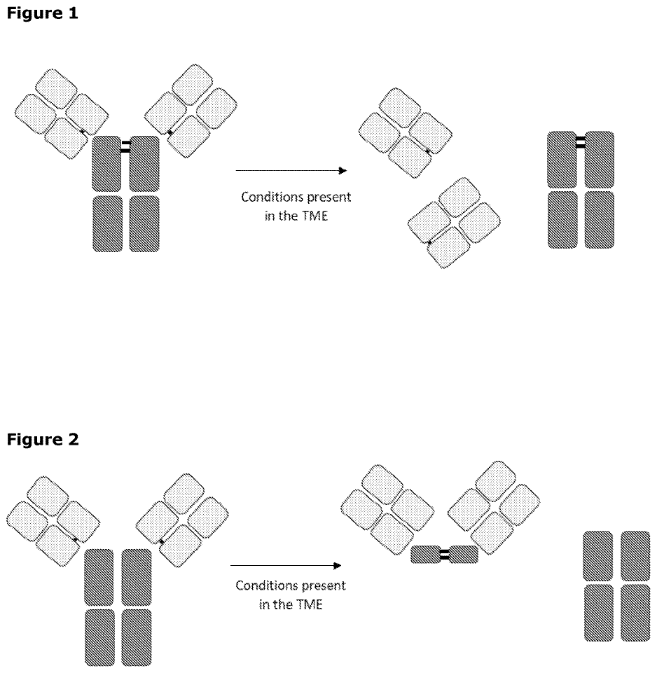

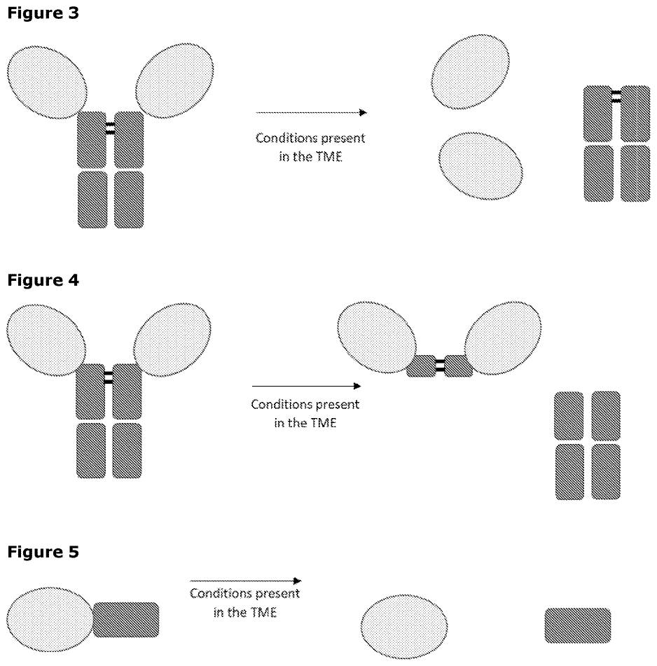

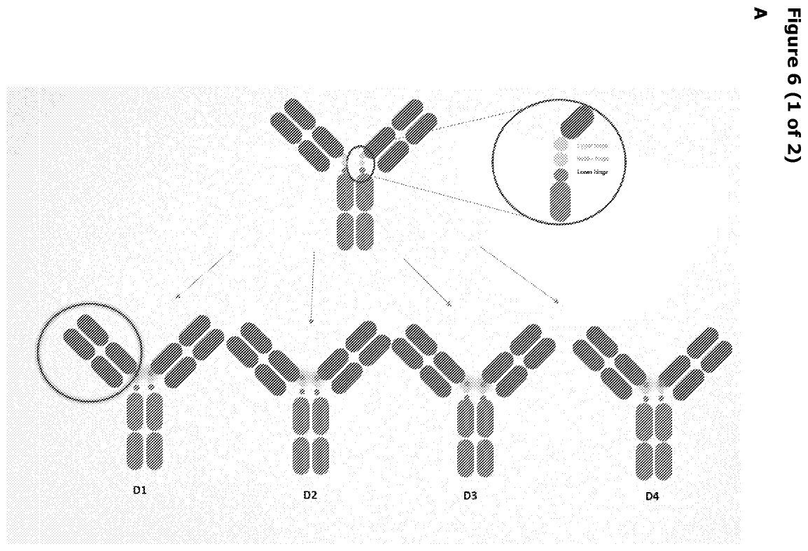

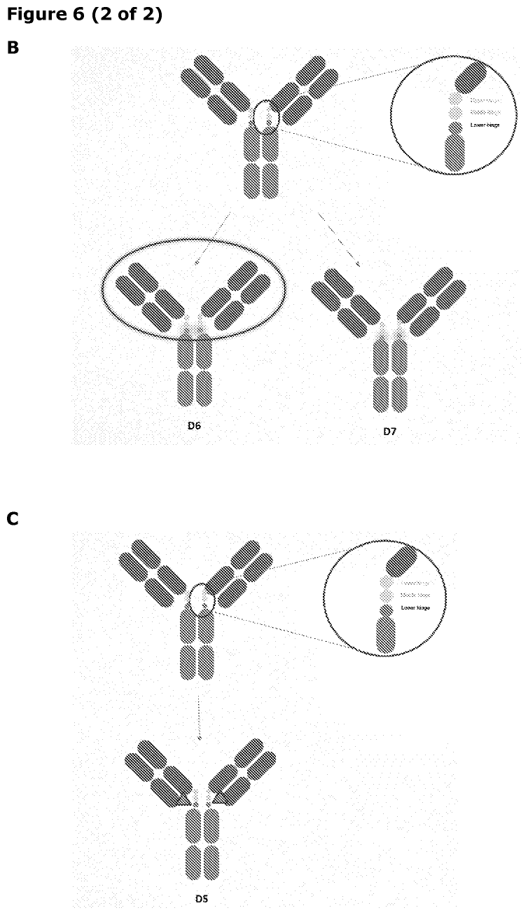

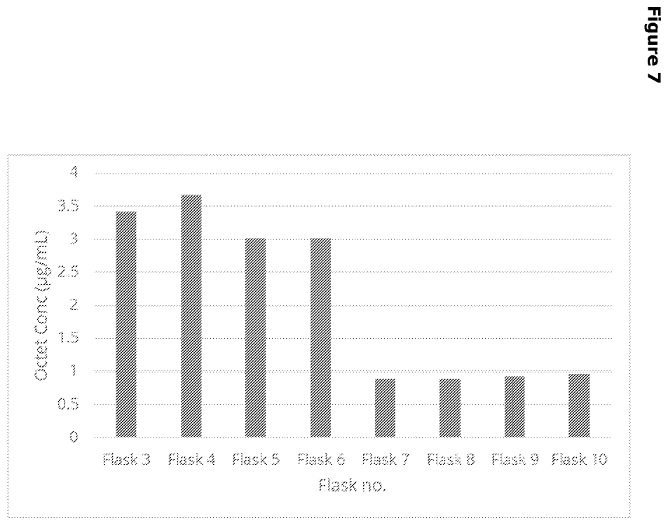

Divalent antibody (F(ab′)2) regions are Fab regions with additional amino acids which are linked to each other (FIG. 2). F(ab′)2 regions may include the entire hinge region that holds the two heavy chains together.

A single-chain variable (scFv) region is a fusion protein of the variable regions of the heavy chain (V_{H}) and light chain (V_{L}) of an antibody. The two chains are connected with a short linker peptide. scFv regions can be in the V_{L}-V_{H} or V_{H}-V_{L} orientation. scFv regions can be monovalent (scFv), or multivalent such as bivalent (e.g. tandem scFv, di-scFv or bi-scFv, diabody), trivalent (e.g. triabody, tribody or tri-scFv), or tetravalent (e.g. tetrabody), pentavalent (e.g. pentabody), hexavalent (e.g. hexabody).

Antibody drug conjugates (ADCs) are molecules comprising an antibody linked to a biologically active cytotoxic drug. For use in preventing or treating conditions characterised by the presence of unwanted cells (e.g. cancer), the antibody is specific to antigens expressed on the unwanted cells and guides the cytotoxic drug to the required location in the body. Examples of ADCs may include but are not limited to trastuzumab emtansine (also called Kadcyla—made by Genentech or Roche), enfortumab vedotin (also called Padcev—made by Astellas or Seattle Genetics), trastuzumab deruxtecan (also called Enhertu—made by AstraZeneca/Daiichi Sankyo), Sacituzumab govitecan (also called Trodelvy—made by Immunomedics), belantamab mafodotin (also called Blenrep—made by GlaxoSmithKline), Tisotumab vedotin-tftv (also called Tivdak—made by Seagen Inc).

A single-domain antibody (sdAb), also known as a nanobody, is an antibody fragment consisting of a single monomeric variable antibody domain. They can be derived from V_{H} domains (e.g. V_{H}H (or V_{H}H) fragments from camelids such as llamas, V_{NAR} fragments from cartilaginous fish), V_{L} domains, or human domain antibodies.

A monoclonal antibody (mAb) is an antibody produced from a cell lineage made by cloning a unique cell, or through a humanised mouse, or recombinant in vitro technology (such as phage display, yeast display or mammalian display), or from in vitro displays (such as ribosome display, mRNA display, cis display). Monoclonal antibodies can have monovalent affinity, binding only to the same epitope (monospecific). In contrast, polyclonal antibodies bind to multiple epitopes (multispecific) and are usually made by several different antibody-secreting plasma cell lineages. In some embodiments, the monoclonal antibodies can be engineered to be multispecific in order to increase the number of epitopes that it binds to.

The therapeutic domain may be monospecific or multispecific, for example bispecific or trispecific. By “multispecific” we include the meaning that the peptide (such as an antibody or antibody fragment) is specific for two or more antigens (for example, bispecific tandem di-scFvs). In one embodiment, the therapeutic domain is not multispecific.

Bicyclic and tricyclic peptides are synthetic short peptides constrained to form two or three loops respectively using a chemical connector compound known as a scaffold, which stabilises their structural geometry. Examples of bicyclic peptides include but are not limited to BT8009 (anti-Nectin 4), BT1718 (anti-MT1-MMP) and BT5528 (anti-EphA2).

In some embodiments, the therapeutic domain has a molecular weight of less than or equal to 1 kDa, less than or equal to 2 kDa, less than or equal to 3 kDa, less than or equal to 4 kDa, less than or equal to 5 kDa, less than or equal to 6 kDa, less than or equal to 7 kDa, less than or equal to 8 kDa, less than or equal to 9 kDa, less than or equal to 10 kDa, less than or equal to 15 kDa, less than or equal to 20 kDa, less than or equal to 25 kDa, less than or equal to 27 kDa, less than or equal to 30 kDa, less than or equal to 35 kDa, less than or equal to 40 kDa, less than or equal to 45 kDa, less than or equal to 50 kDa, less than or equal to 55 kDa, less than or equal to 60 kDa, less than or equal to 65 kDa, less than or equal to 70 kDa, less than or equal to 75 kDa, less than or equal to 80 kDa, less than or equal to 85 kDa, less than or equal to 90 kDa, less than or equal to 95 kDa, less than or equal to 100 kDa.

In one embodiment, the cleaved therapeutic domain has a molecular weight of no more than 60 kDa, no more than 55 kDa, no more than 50 kDa, no more than 40 kDa, no more than 30 kDa, no more than 25 kDa, no more than 20 kDa, no more than 20 kDa, no more than 15 kDa.

Preferably, the agent is selected from the group: i. a monoclonal antibody; ii. a polyclonal antibody; iii. diabody; iv. triabody; v. tetrabody; vi. pentabody; vii. hexabody; viii. an antibody drug conjugate; ix. a bispecific peptide, such as a bispecific antibody; and/or x. a multispecific peptide, such as a multispecific antibody.

Particularly preferred agents and/or therapeutic domains include antibodies that are derived from anti-epidermal growth factor receptor (EGFR) antibodies such as Cetuximab-derived fragments, Panitumumab-derived fragments, Zalutumumab-derived fragments; from anti-Her2 antibodies such as Trastuzumab-derived fragments, Pertuzumab-derived fragments; from anti-CD20 antibodies such as Rituximab-derived fragments; from anti-CD22 antibodies such as Inotuzumab-derived fragments; from anti-CD70 antibodies; from anti-CD33 antibodies such as hp67.6-derived fragments, Gemtuzumab-derived fragment; from anti-MUC1 antibodies such as GP1.4-derived fragments, SM3-derived fragments; from anti-CD40 antibodies, from anti-CD74 antibodies, from anti-P-cadherin antibodies, from anti-EpCAM antibodies; from anti-CD138 antibodies; from anti-E-cadherin antibodies; from anti-CEA antibodies such as M5A; fromanti-FGFR3 antibodies; from anti-PSMA antibodies such as J591; from anti-CTLA4 antibodies, such as Ipilimumab-derived fragments, Tremelimumab-derived fragments; from anti-PD1 antibodies, such as Pembrolizumab-derived fragments, Nivolumab-derived fragments; and from anti-PDL1 antibodies, such as Atezolizumab-derived fragments; from anti-VEGF (vascular endothelial growth factor) antibodies, such as Bevacizumab.

By “antibodies that are derived from” and “antibody-derived fragment”, we include the meaning of antibodies or fragments of antibodies that comprise parts or modified parts of the parent antibody from which they are derived. For example, an antibody-derived fragment may have the same of similar sequences as parts of the parent antibody. In some embodiments, an antibody-derived fragment has at least 70%, 71%, 72%, 73%, 74%, 75%, 76%, 77%, 78% or 79% sequence identity to a part of the antibody sequence.

In some embodiments, the agents provided herein in an uncleaved state comprise a Trastuzumab-derived fragment that is at least 90%, 91%, 92%, 93%, 94%, 95%, 96%, 97%, 98% or 99% identical to a sequence selected from the group consisting of:

The cleavable domains and the cleavage sites of the below described engineered designs D1-D9 and D12-D19 have been emboldened and underlined, respectively.

D1: Engineered trastuzumab antibody design 1
(SEQ ID NO: 1)
EVQLVESGGGLVQPGGSLRLSCAASGFNIKDTYIHWVRQAPGKGLEWVARIYPTNGYTRYADSVKGRFTI
SADTSKNTAYLQMNSLRAEDTAVYYCSRWGGDGFYAMDYWGQGTLVTVSSASTKGPSVFPLAPSSKSTS
GGTAALGCLVKDYFPEPVTVSWNSGALTSGVHTFPAVLQSSGLYSLSSVVTVPSSSLGTQTYICNVNHKP
SNTKVDKKVEPKSCGSLSGRSDNHDKTHTCPPCPAPELLGGPSVFLFPPKPKDTLMISRTPEVTCVVV
DVSHEDPEVKFNWYVDGVEVHNAKTKPREEQYNSTYRVVSVLTVLHQDWLNGKEYKCKVSNKALPAPIE
KTISKAKGQPREPQVYTLPPSREEMTKNQVSLTCLVKGFYPSDIAVEWESNGQPENNYKTTPPVLDSDGSF
FLYSKLTVDKSRWQQGNVFSCSVMHEALHNHYTQKSLSLSPGK
D2: Engineered trastuzumab antibody design 2
(SEQ ID NO: 2)
EVQLVESGGGLVQPGGSLRLSCAASGFNIKDTYIHWVRQAPGKGLEWVARIYPTNGYTRYADSVKGRFTI
SADTSKNTAYLQMNSLRAEDTAVYYCSRWGGDGFYAMDYWGQGTLVTVSSASTKGPSVFPLAPSSKSTS
GGTAALGCLVKDYFPEPVTVSWNSGALTSGVHTFPAVLQSSGLYSLSSVVTVPSSSLGTQTYICNVNHKP
SNTKVDKKVEPKSCLSGRSDNHDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGKEYKCKVSNKALPAPIEKTIS
KAKGQPREPQVYTLPPSREEMTKNQVSLTCLVKGFYPSDIAVEWESNGQPENNYKTTPPVLDSDGSFFLYS
KLTVDKSRWQQGNVFSCSVMHEALHNHYTQKSLSLSPGK
D3: Engineered trastuzumab antibody design 3
(SEQ ID NO: 3)
EVQLVESGGGLVQPGGSLRLSCAASGFNIKDTYIHWVRQAPGKGLEWVARIYPTNGYTRYADSVKGRFTI
SADTSKNTAYLQMNSLRAEDTAVYYCSRWGGDGFYAMDYWGQGTLVTVSSASTKGPSVFPLAPSSKSTS
GGTAALGCLVKDYFPEPVTVSWNSGALTSGVHTFPAVLQSSGLYSLSSVVTVPSSSLGTQTYICNVNHKP
SNTKVDKKVEPKSCGSLSGRSDNHGSDKTHTCPPCPAPELLGGPSVFLFPPKPKDTLMISRTPEVTCV
VVDVSHEDPEVKFNWYVDGVEVHNAKTKPREEQYNSTYRVVSVLTVLHQDWLNGKEYKCKVSNKALPAP
IEKTISKAKGQPREPQVYTLPPSREEMTKNQVSLTCLVKGFYPSDIAVEWESNGQPENNYKTTPPVLDSDG
SFFLYSKLTVDKSRWQQGNVFSCSVMHEALHNHYTQKSLSLSPGK
D4: Engineered trastuzumab antibody design 4
(SEQ ID NO: 4)
EVQLVESGGGLVQPGGSLRLSCAASGFNIKDTYIHWVRQAPGKGLEWVARIYPTNGYTRYADSVKGRFTI
SADTSKNTAYLQMNSLRAEDTAVYYCSRWGGDGFYAMDYWGQGTLVTVSSASTKGPSVFPLAPSSKSTS
GGTAALGCLVKDYFPEPVTVSWNSGALTSGVHTFPAVLQSSGLYSLSSVVTVPSSSLGTQTYICNVNHKP
SNTKVDKKVEPKSCLSGRSDNHCPPCPAPELLGGPSVFLFPPKPKDTLMISRTPEVTCVVVDVSHEDPE
VKFNWYVDGVEVHNAKTKPREEQYNSTYRVVSVLTVLHQDWLNGKEYKCKVSNKALPAPIEKTISKAKG
QPREPQVYTLPPSREEMTKNQVSLTCLVKGFYPSDIAVEWESNGQPENNYKTTPPVLDSDGSFFLYSKLTV
DKSRWQQGNVFSCSVMHEALHNHYTQKSLSLSPGK
D5: Engineered trastuzumab antibody design 5
(SEQ ID NO: 5)
EVQLVESGGGLVQPGGSLRLSCAASGFNIKDTYIHWVRQAPGKGLEWVARIYPTNGYTRYADSVKGRFTI
SADTSKNTAYLQMNSLRAEDTAVYYCSRWGGDGFYAMDYWGQGTLVTVSSASTKGPSVFPLAPSSKSTS
GGTAALGCLVKDYFPEPVTVSWNSGALTSGVHTFPAVLQSSGLYSLSSVVTVPSSSLGTQTYICNVNHKP
SNTKVDKKVEPKSCLSGRSDNHTCPPCPAPELLGGPSVFLFPPKPKDTLMISRTPEVTCVVVDVSHEDP
EVKFNWYVDGVEVHNAKTKPREEQYNSTYRVVSVLTVLHQDWLNGKEYKCKVSNKALPAPIEKTISKAKG
QPREPQVYTLPPSREEMTKNQVSLTCLVKGFYPSDIAVEWESNGQPENNYKTTPPVLDSDGSFFLYSKLTV
DKSRWQQGNVFSCSVMHEALHNHYTQKSLSLSPGK
D6: Engineered trastuzumab antibody design 6
(SEQ ID NO: 6)
EVQLVESGGGLVQPGGSLRLSCAASGFNIKDTYIHWVRQAPGKGLEWVARIYPTNGYTRYADSVKGRFTI
SADTSKNTAYLQMNSLRAEDTAVYYCSRWGGDGFYAMDYWGQGTLVTVSSASTKGPSVFPLAPSSKSTS
GGTAALGCLVKDYFPEPVTVSWNSGALTSGVHTFPAVLQSSGLYSLSSVVTVPSSSLGTQTYICNVNHKP
SNTKVDKKVEPKSCDKTHTCPPCPAPLSGRSDNHELLGGPSVFLFPPKPKDTLMISRTPEVTCVVVDVS
HEDPEVKFNWYVDGVEVHNAKTKPREEQYNSTYRVVSVLTVLHQDWLNGKEYKCKVSNKALPAPIEKTIS
KAKGQPREPQVYTLPPSREEMTKNQVSLTCLVKGFYPSDIAVEWESNGQPENNYKTTPPVLDSDGSFFLYS
KLTVDKSRWQQGNVFSCSVMHEALHNHYTQKSLSLSPGK
D7: Engineered trastuzumab antibody design 7
(SEQ ID NO: 7)
EVQLVESGGGLVQPGGSLRLSCAASGFNIKDTYIHWVRQAPGKGLEWVARIYPTNGYTRYADSVKGRFTI
SADTSKNTAYLQMNSLRAEDTAVYYCSRWGGDGFYAMDYWGQGTLVTVSSASTKGPSVFPLAPSSKSTS
GGTAALGCLVKDYFPEPVTVSWNSGALTSGVHTFPAVLQSSGLYSLSSVVTVPSSSLGTQTYICNVNHKP
SNTKVDKKVEPKSCDKTHTCPPCPAPLSGRSDNHFLFPPKPKDTLMISRTPEVTCVVVDVSHEDPEVKF
NWYVDGVEVHNAKTKPREEQYNSTYRVVSVLTVLHQDWLNGKEYKCKVSNKALPAPIEKTISKAKGQPRE
PQVYTLPPSREEMTKNQVSLTCLVKGFYPSDIAVEWESNGQPENNYKTTPPVLDSDGSFFLYSKLTVDKSR
WQQGNVFSCSVMHEALHNHYTQKSLSLSPGK
D9: Engineered trastuzumab antibody design 9 [ADAM10]
(SEQ ID NO: 8)
EVQLVESGGGLVQPGGSLRLSCAASGFNIKDTYIHWVRQAPGKGLEWVARIYPTNGYTRYADSVKGRFTI
SADTSKNTAYLQMNSLRAEDTAVYYCSRWGGDGFYAMDYWGQGTLVTVSSASTKGPSVFPLAPSSKSTS
GGTAALGCLVKDYFPEPVTVSWNSGALTSGVHTFPAVLQSSGLYSLSSVVTVPSSSLGTQTYICNVNHKP
SNTKVDKKVEPKSCGSPRAEALKGGGSASDKTHTCPPCPAPELLGGPSVFLFPPKPKDTLMISRTPEV
TCVVVDVSHEDPEVKFNWYVDGVEVHNAKTKPREEQYNSTYRVVSVLTVLHQDWLNGKEYKCKVSNKAL
PAPIEKTISKAKGQPREPQVYTLPPSREEMTKNQVSLTCLVKGFYPSDIAVEWESNGQPENNYKTTPPVLD
SDGSFFLYSKLTVDKSRWQQGNVFSCSVMHEALHNHYTQKSLSLSPGK
D17: Engineered anti-EGFR antibody design 17 [VHH 7D12-FC]
(SEQ ID NO: 9)
QVQLQESGGGLVQPGGSLRLSCAASGRTFSSYAMGWFRQAPGKQREFVAAIRWSGGYTYYTDSVKGRF
TISRDNAKTTVYLQMNSLKPEDTAVYYCAATYLSSDYSRYALPQRPLDYDYWGQGTQVTVSSSLSGRSD
NHGSASDKTHTCPPCPAPELLGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQGNV
FSCSVMHEALHNHYTQKSLSLSPGK
D18: Engineered trastuzumab antibody design 18 [anti-Her2 ScFv-FC]
(SEQ ID NO: 10)
DIQMTQSPSSLSASVGDRVTITCRASQDVNTAVAWYQQKPGKAPKLLIYSASFLYSGVPSRFSGSRSGTD
FTLTISSLQPEDFATYYCQQHYTTPPTFGQGTKVEIKGGGGSGGGGSGGGGSEVQLVESGGGLVQPGGS
LRLSCAASGFNIKDTYIHWVRQAPGKGLEWVARIYPTNGYTRYADSVKGRFTISADTSKNTAYLQMNSLR
AEDTAVYYCSRWGGDGFYAMDYWGQGTLVTVSSGGGSGGGSLSGRSDNHGSASDKTHTCPPCPA
PELLGGPSVFLFPPKPKDTLMISRTPEVTCVVVDVSHEDPEVKFNWYVDGVEVHNAKTKPREEQYNSTYR
VVSVLTVLHQDWLNGKEYKCKVSNKALPAPIEKTISKAKGQPREPQVYTLPPSREEMTKNQVSLTCLVKGF
YPSDIAVEWESNGQPENNYKTTPPVLDSDGSFFLYSKLTVDKSRWQQGNVFSCSVMHEALHNHYTQKSL
SLSPGK

In some embodiments, the agents provided herein in an uncleaved state comprise a Cetuximab-derived fragment that is at least 90%, 91%, 92%, 93%, 94%, 95%, 96%, 97%, 98% or 99% identical to the sequence:

D12: Engineered cetuximab antibody design 12 (SEQ ID NO: 11):
QVQLKQSGPGLVQPSQSLSITCTVSGFSLTNYGVHWVRQSPGKGLEWLGVIWSGGNTDYNTPFTSRLSI
NKDNSKSQVFFKMNSLQSNDTAIYYCARALTYYDYEFAYWGQGTLVTVSAASTKGPSVFPLAPSSKSTSG
GTAALGCLVKDYFPEPVTVSWNSGALTSGVHTFPAVLQSSGLYSLSSVVTVPSSSLGTQTYICNVNHKPS
NTKVDKKVEPKSCGSLSGRSDNHGSASDKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKALPA
PIEKTISKAKGQPREPQVYTLPPSREEMTKNQVSLTCLVKGFYPSDIAVEWESNGQPENNYKTTPPVLDSD
GSFFLYSKLTVDKSRWQQGNVFSCSVMHEALHNHYTQKSLSLSPGK

In some embodiments, the agents provided herein in an uncleaved state comprise a Bevacizumab-derived fragment that is at least 90%, 91%, 92%, 93%, 94%, 95%, 96%, 97%, 98% or 99% identical to the sequence:

D13: Engineered bevacizumab antibody design 13 (SEQ ID NO: 12):
EVQLVESGGGLVQPGGSLRLSCAASGYTFTNYGMNWVRQAPGKGLEWVGWINTYTGEPTYAADFKRRFT
FSLDTSKSTAYLQMNSLRAEDTAVYYCAKYPHYYGSSHWYFDVWGQGTLVTVSSASTKGPSVFPLAPSSK
STSGGTAALGCLVKDYFPEPVTVSWNSGALTSGVHTFPAVLQSSGLYSLSSVVTVPSSSLGTQTYICNVN
HKPSNTKVDKKVEPKSCGSLSGRSDNHGSASDKTHTCPPCPAPELLGGPSVFLFPPKPKDTLMISRTP
EVTCVVVDVSHEDPEVKFNWYVDGVEVHNAKTKPREEQYNSTYRVVSVLTVLHQDWLNGKEYKCKVSNK
ALPAPIEKTISKAKGQPREPQVYTLPPSREEMTKNQVSLTCLVKGFYPSDIAVEWESNGQPENNYKTTPPVL
DSDGSFFLYSKLTVDKSRWQQGNVFSCSVMHEALHNHYTQKSLSLSPGK

In some embodiments, the agents provided herein in an uncleaved state comprise a Pembrolizumab-derived fragment that is at least 90%, 91%, 92%, 93%, 94%, 95%, 96%, 97%, 98% or 99% identical to a sequence selected from the group consisting of:

D14a: Engineered pembrolizumab antibody design 14a (SEQ ID NO: 13):
QVQLVQSGVEVKKPGASVKVSCKASGYTFTNYYMYWVRQAPGQGLEWMGGINPSNGGTNFNEKFKNRV
TLTTDSSTTTAYMELKSLQFDDTAVYYCARRDYRFDMGFDYWGQGTTVTVSSASTKGPSVFPLAPCSRST
SESTAALGCLVKDYFPEPVTVSWNSGALTSGVHTFPAVLQSSGLYSLSSVVTVPSSSLGTKTYTCNVDHKP
SNTKVDKRVESKYGSLSGRSDNHGSGPPCPPCPAPEFLGGPSVFLFPPKPKDTLMISRTPEVTCVVVD
VSQEDPEVQFNWYVDGVEVHNAKTKPREEQFNSTYRVVSVLTVLHQDWLNGKEYKCKVSNKGLPSSIEK
TISKAKGQPREPQVYTLPPSQEEMTKNQVSLTCLVKGFYPSDIAVEWESNGQPENNYKTTPPVLDSDGSFF
LYSRLTVDKSRWQEGNVFSCSVMHEALHNHYTQKSLSLSLGK

D14b: Engineered pembrolizumab antibody design 14b (SEQ ID NO: 14):

QVQLVQSGVEVKKPGASVKVSCKASGYTFTNYYMYWVRQAPGQGLEWMGGINPSNGGTNFNEKFKNRV
TLTTDSSTTTAYMELKSLQFDDTAVYYCARRDYRFDMGFDYWGQGTTVTVSSASTKGPSVFPLAPCSRST
SESTAALGCLVKDYFPEPVTVSWNSGALTSGVHTFPAVLQSSGLYSLSSVVTVPSSSLGTKTYTCNVDHKP
SNTKVDKRVESKYGPPGSLSGRSDNHGSCPPCPAPEFLGGPSVFLFPPKPKDTLMISRTPEVTCVVVD
VSQEDPEVQFNWYVDGVEVHNAKTKPREEQFNSTYRVVSVLTVLHQDWLNGKEYKCKVSNKGLPSSIEK
TISKAKGQPREPQVYTLPPSQEEMTKNQVSLTCLVKGFYPSDIAVEWESNGQPENNYKTTPPVLDSDGSFF
LYSRLTVDKSRWQEGNVFSCSVMHEALHNHYTQKSLSLSLGK

In some embodiments, the agents provided herein in an uncleaved state comprise a M5A-derived fragment that is at least 90%, 91%, 92%, 93%, 94%, 95%, 96%, 97%, 98% or 99% identical to the sequence:

D15: Engineered M5A antibody design 15 (SEQ ID NO: 15):
EVQLVESGGGLVQPGGSLRLSCAASGFNIKDTYMHWVRQAPGKGLEWVARIDPANGNSKYADSVKGRF
TISADTSKNTAYLQMNSLRAEDTAVYYCAPFGYYVSDYAMAYWGQGTLVTVSSASTKGPSVFPLAPSSKS
TSGGTAALGCLVKDYFPEPVTVSWNSGALTSGVHTFPAVLQSSGLYSLSSVVTVPSSSLGTQTYICNVNH
KPSNTKVDKKVEPKSCGSLSGRSDNHGSASDKTHTCPPCPAPELLGGPSVFLFPPKPKDTLMISRTPE
VTCVVVDVSHEDPEVKFNWYVDGVEVHNAKTKPREEQYNSTYRVVSVLTVLHQDWLNGKEYKCKVSNK
ALPAPIEKTISKAKGQPREPQVYTLPPSREEMTKNQVSLTCLVKGFYPSDIAVEWESNGQPENNYKTTPPVL
DSDGSFFLYSKLTVDKSRWQQGNVFSCSVMHEALHNHYTQKSLSLSPGK

In some embodiments, the agents provided herein in an uncleaved state comprise an Ipilimumab-derived fragment that is at least 90%, 91%, 92%, 93%, 94%, 95%, 96%, 97%, 98% or 99% identical to the sequence:

D16: Engineered Ipilimumab antibody design 16 (SEQ ID NO: 16):
QVQLVESGGGVVQPGRSLRLSCAASGFTFSSYTMHWVRQAPGKGLEWVTFISYDGNNKYYADSVKGRFT
ISRDNSKNTLYLQMNSLRAEDTAIYYCARTGWLGPFDYWGQGTLVTVSSASTKGPSVFPLAPSSKSTSGG
TAALGCLVKDYFPEPVTVSWNSGALTSGVHTFPAVLQSSGLYSLSSVVTVPSSSLGTQTYICNVNHKPSNT
KVDKRVEPKSCGSLSGRSDNHGSASDKTHTCPPCPAPELLGGPSVFLFPPKPKDTLMISRTPEVTCVV
VDVSHEDPEVKFNWYVDGVEVHNAKTKPREEQYNSTYRVVSVLTVLHQDWLNGKEYKCKVSNKALPAPI
EKTISKAKGQPREPQVYTLPPSREEMTKNQVSLTCLVKGFYPSDIAVEWESNGQPENNYKTTPPVLDSDG
SFFLYSKLTVDKSRWQQGNVFSCSVMHEALHNHYTQKSLSLSPGK

In some embodiments, the therapeutic domain is monovalent, bivalent, trivalent or multivalent (e.g. tetravalent). A monovalent therapeutic domain may have an affinity for one epitope, antigen, or strain of microorganism; whereas, a multivalent (e.g. bivalent, trivalent, tetravalent) therapeutic domain may have an affinity for various epitopes, antigens, or strains of microorganisms. In one embodiment, the therapeutic domain is not multivalent.

In one embodiment, the therapeutic domain targets or guides the agent to unwanted cells. By “therapeutic domain targeting the agent”, we include the meaning that that therapeutic domain may by a specific binding partner of an entity expressed by or associated with unwanted cells.

In one embodiment, the therapeutic domain is a specific binding partner of an entity expressed by or associated with a target cell or a target tissue. Typically, the expressed entity is expressed selectively on the unwanted cell. For example, the abundance of the expressed entity is typically 10 or 100 or 500 or 1,000 or 5,000 or 10,000 higher on the unwanted cell than on other cells within the body to be treated. However, as mentioned below, the cleavage site provides additional specificity on where the therapeutic domain is released and so the binding partner may bind an entity that is similarly or under-expressed on unwanted cells relative to other cells within the body.

Most preferably, however, the therapeutic domain is a specific binding partner of an entity expressed by or associated with unwanted cells, as opposed to any other cells.

By “binding partner” we include the meaning of a molecule that binds to a target entity expressed by a particular cell. Preferably, the binding partner binds selectively to that entity. Antibodies that bind specifically to a target (which can be an epitope) are antibodies which bind to that target with greater affinity, avidity, more readily, and/or with greater duration than to other unrelated targets or molecules.

As will be appreciated, the specificity of an antibody for its target can be determined and/or defined based on affinity measurements. Affinity (K_{D}), expressed by the equilibrium constant for dissociation between antigen and antibody, is a measure of the strength of binding between the epitope and the antigen binding site on the antibody: a smaller K_{D} value indicates that the binding strength between antigen binding molecules is stronger (alternatively, affinity can also be expressed as an affinity constant (K_{A}), which is 1/K_{D}). As will be apparent to those skilled in the art, affinity can be determined by any method known in the art and described herein. Any K_{D} value greater than 1×10⁻⁶ M is generally considered to indicate non-specific binding.

For example, it is preferred if the binding partner has a K_{D} value in respect of the target which is at least five or ten times lower (i.e. higher affinity) than for at least one other entity expressed by another cell (e.g. a normal cell type), and preferably more than 100 or 500 times lower. More preferably, the binding partner of that entity has a K_{D} value more than 1000 or 5000 times lower than for at least one other entity expressed by another cell (e.g. normal cell type). Binding specificity of the binding molecule can be determined experimentally by methods known in the art. Such methods comprise but are not limited to Biophysical Biolayer interferometry (BLI), isothermal titration calorimetry (ITC), Western blots, enzyme-linked immunosorbent assay (ELISA), radioimmunoassay (RIA), electrochemiluminescence (ECL), immunoradiometric assay (IRMA), Enzyme immunoassay (EIA), and surface plasmon resonance (SPR).

Typically, the binding partner is one that binds to an entity that is present or accessible to the binding partner in significantly greater concentrations in or on or around unwanted cells than in any normal cells of the host. For example, the binding partner may bind to a tumour associated antigen which is expressed on the cell membrane. In another embodiment, the binding partner may bind an entity that is similarly or under-expressed on unwanted cells relative to other cells within the body. In one embodiment, the entity is naturally occurring or endogenously expressed.

Preferably, the target entity is an antigen at the unwanted cell surface, i.e. a cell surface marker. The diameter of solid tumours can vary from small (such as 1 centimetre (cm)) to large (such as bigger than 10 cm). Solid tumours are 3D structures, with periphery and interior parts. Target entities may be located at the periphery (e.g. surface) and/or at the interior (e.g. epicentre) of the tumour. The epicentre is considered the inner part of the interior of tumour and may be at least 0.5 cm, at least 1 cm, at least 2 cm, at least 3 cm, at least 4 cm, at least 5 cm, at least 6 cm, at least 7 cm away from the surface of the tumour (Sopik and Narod, 2018. The relationship between tumour size, nodal status and distant metastases: on the origins of breast cancer. Breast Cancer Res Treat. 2018; 170(3): 647-656).

“Unwanted cells” include cells whose presence in a host or patient is undesired, such as tumour cells or other disease-causing cells.

In some embodiments, the therapeutic domain may be any compound or part that specifically binds (in a non-immune sense) to an entity expressed by unwanted cells or otherwise becomes associated with unwanted cells. Thus, the therapeutic domain may be any of: i. a T-cell receptor (TCR) domain; or ii. a receptor domain; or iii. a receptor mimic domain; or iv. a cytokine; or v. a hormone; or vi. a growth factor; or vii. a peptide; or viii. a derivative of a peptide.

In one embodiment, the therapeutic domain may bind an intracellular target. In another embodiment, the therapeutic domain may bind an extracellular target. In some embodiments, the therapeutic domain may bind both intracellular and extracellular targets.

In another embodiment, the therapeutic domain does not target the agent to the unwanted cells.

Particularly useful therapeutic domains could include peptides or derivatives of peptides such as MYC (also known as c-Myc) inhibitors (e.g. H1 peptide, OmoMYC), HOX (homeobox) inhibitors (e.g. HRX9, HTL-001 (HOX Therapeutics Ltd.)).

Particularly useful therapeutic domains could include cytokines such as IGF (insulin-like growth factor), EGF (epidermal growth factor), VEGF (vascular endothelial growth factor), IL (interleukin)-2, IL-6, IL-4, or HGF (hepatocyte growth factor, scatter factor, SF, hepatopoeitin A).

Insulin like growth factors (IGF-1 and IGF-11) are preferentially taken up by malignant cells and so may be used to target tumour cells. Similarly, EGF can be used to target malignant cells which upregulate the EGF receptor. Also, tumour associated blood vessels overexpress VEGF receptor and so can be targeted by the family of VEGF growth factors. Myeloma cells express IL-6 receptor and also secrete IL-6 which acts in an autocrine fashion to stimulate cell proliferation. Thus IL-6 may be used as a therapeutic domain for myeloma. Myeloma cells express IL-6 receptor and also secrete IL-6 which acts in an autocrine fashion to stimulate cell proliferation. Thus IL-6 may be used as a therapeutic domain for myeloma. In another example, the therapeutic domain is melanoma stimulating hormone (MSH) which binds to the MSH receptor which is expressed in high numbers in melanoma cells.

In an embodiment, the therapeutic domain binds to an antigen expressed by the unwanted cell selected from a list comprising: CEA (anticarcinoembryonic antigen); Her2/Neu; CD22 (sialic acid binding Ig-like lectin 2, SIGLEC2, SIGLEC-2, B-lymphocyte cell adhesion molecule, BL-CAM, Leu-14); EPCAM (epithelial cell adhesion molecule, tumour-associated calcium signal transducer 1, TACSTD1, gastrointestinal tumour-associated protein 2, GA733-2, epithelial glycoprotein 2, EGP-2, epithelial cell adhesion molecule, Ep-CAM, KSA, KS1/4 antigen, M4S, tumour antigen 17-1A, EpCAM, CD326); EGFR (epidermal growth factor receptor, receptor tyrosine-protein kinase erbB-1, ERBB1, HER1, HER-1, ERBB); PMSA; CTLA-4 (cytotoxic T lymphocyte-associated antigen 4, CTLA4, CD152) CD30; CD20; CD33 (sialic acid binding Ig-like lectin 3, SIGLEC3, SIGLEC-3, gpG7, p67); CD80 (B7-1, CD28LG1); CD86 (B7-2, CD28LG2); CD2; CA125; Carbonic Anhydrase IX; CD70 (tumour necrosis factor superfamily member 7, TNFSF7, CD27LG, CD27L); CD74 (major histocompatibility class II invariant chain, MH2); CD56; CD40 (tumour necrosis factor receptor superfamily member 5, TNFRSF5, p50); CD19; c-met/HGFR; TRAIL-R1; DR5; PD-1; PD1L; IGF-1R; VEGF; VEGF-R2; Prostate stem cell antigen (PSCA); MUC1 sialylated carbohydrate, tumour-associated (CA242, cancer antigen 242); CanAg; Mesothelin; P-cadherin; Myostatin (GDF8); Cripto (TDGF1); ACVRLi/ALK1; MUC5AC; CEACAM ((carcinoembryonic antigen-related cell adhesion molecules); CD137; CXCR4; Neuropilin; Glypicans; HER3/EGFR; PDGFRa (platelet-derived growth factor receptor alpha subunit, PDGFR2, CD140a); EphA2; CD138. Preferably, therapeutic domain binds to an antigen selected from a list comprising Her2, VEGF, PD-1, PMSA, CEA, CTLA-4 or EGFR.

In some embodiments, the therapeutic domain provided herein comprise a Trastuzumab-derived fragment that is at least 90%, 91%, 92%, 93%, 94%, 95%, 96%, 97%, 98% or 99% identical to a sequence selected from the group consisting of:

Therapeutic domain of Engineered trastuzumab designs 1-5 and 8-10
(SEQ ID NO: 17)
EVQLVESGGGLVQPGGSLRLSCAASGFNIKDTYIHWVRQAPGKGLEWVARIYPTNGYTRYADSVKGRFTI
SADTSKNTAYLQMNSLRAEDTAVYYCSRWGGDGFYAMDYWGQGTLVTVSSASTKGPSVFPLAPSSKSTS
GGTAALGCLVKDYFPEPVTVSWNSGALTSGVHTFPAVLQSSGLYSLSSVVTVPSSSLGTQTYICNVNHKP
SNTKVDKKVEPKSC
Therapeutic domain of Engineered trastuzumab designs 6 & 7
(SEQ ID NO: 18)
EVQLVESGGGLVQPGGSLRLSCAASGFNIKDTYIHWVRQAPGKGLEWVARIYPTNGYTRYADSVKGRFTI
SADTSKNTAYLQMNSLRAEDTAVYYCSRWGGDGFYAMDYWGQGTLVTVSSASTKGPSVFPLAPSSKSTS
GGTAALGCLVKDYFPEPVTVSWNSGALTSGVHTFPAVLQSSGLYSLSSVVTVPSSSLGTQTYICNVNHKP
SNTKVDKKVEPKSCDKTHTCPPCPAP
Therapeutic domain of Engineered trastuzumab design 17
(SEQ ID NO: 19)
QVQLQESGGGLVQPGGSLRLSCAASGRTFSSYAMGWFRQAPGKQREFVAAIRWSGGYTYYTDSVKGRF
TISRDNAKTTVYLQMNSLKPEDTAVYYCAATYLSSDYSRYALPQRPLDYDYWGQGTQVTVSSS
Therapeutic domain of Engineered trastuzumab design 18
(SEQ ID NO: 20)
DIQMTQSPSSLSASVGDRVTITCRASQDVNTAVAWYQQKPGKAPKLLIYSASFLYSGVPSRFSGSRSGTD
FTLTISSLQPEDFATYYCQQHYTTPPTFGQGTKVEIKGGGGSGGGGSGGGGSEVQLVESGGGLVQPGGS
LRLSCAASGFNIKDTYIHWVRQAPGKGLEWVARIYPTNGYTRYADSVKGRFTISADTSKNTAYLQMNSLR
AEDTAVYYCSRWGGDGFYAMDYWGQGTLVTVSS

In some embodiments, the therapeutic domain provided herein comprise a Cetuximab-derived fragment that is at least 90%, 91%, 92%, 93%, 94%, 95%, 96%, 97%, 98% or 99% identical to the sequence:

Therapeutic domain of Engineered cetuximab design 12
(SEQ ID NO: 21)
QVQLKQSGPGLVQPSQSLSITCTVSGFSLTNYGVHWVRQSPGKGLEWLGVIWSGGNTDYNTPFTSRLSI
NKDNSKSQVFFKMNSLQSNDTAIYYCARALTYYDYEFAYWGQGTLVTVSAASTKGPSVFPLAPSSKSTSG
GTAALGCLVKDYFPEPVTVSWNSGALTSGVHTFPAVLQSSGLYSLSSVVTVPSSSLGTQTYICNVNHKPS
NTKVDKKVEPKSC

In some embodiments, the therapeutic domain provided herein comprise a Bevacizumab-derived fragment that is at least 90%, 91%, 92%, 93%, 94%, 95%, 96%, 97%, 98% or 99% identical to the sequence:

Therapeutic domain of Engineered bevacizumab design 13
(SEQ ID NO: 22)
EVQLVESGGGLVQPGGSLRLSCAASGYTFTNYGMNWVRQAPGKGLEWVGWINTYTGEPTYAADFKRRFT
FSLDTSKSTAYLQMNSLRAEDTAVYYCAKYPHYYGSSHWYFDVWGQGTLVTVSSASTKGPSVFPLAPSSK
STSGGTAALGCLVKDYFPEPVTVSWNSGALTSGVHTFPAVLQSSGLYSLSSVVTVPSSSLGTQTYICNVN
HKPSNTKVDKKVEPKSC

In some embodiments, the therapeutic domain provided herein comprise a Pembrolizumab-derived fragment that is at least 90%, 91%, 92%, 93%, 94%, 95%, 96%, 97%, 98% or 99% identical to sequence:

Therapeutic domain of Engineered pembrolizumab designs 14a and 14b
(SEQ ID NO: 23)
QVQLVQSGVEVKKPGASVKVSCKASGYTFTNYYMYWVRQAPGQGLEWMGGINPSNGGTNFNEKFKNRV
TLTTDSSTTTAYMELKSLQFDDTAVYYCARRDYRFDMGFDYWGQGTTVTVSSASTKGPSVFPLAPCSRST
SESTAALGCLVKDYFPEPVTVSWNSGALTSGVHTFPAVLQSSGLYSLSSVVTVPSSSLGTKTYTCNVDHKP
SNTKVDKRVESKY

In some embodiments, the therapeutic domain provided herein comprise a M5A-derived fragment that is at least 90%, 91%, 92%, 93%, 94%, 95%, 96%, 97%, 98% or 99% identical to the sequence:

Therapeutic domain of Engineered M5A design 15
(SEQ ID NO: 24)
EVQLVESGGGLVQPGGSLRLSCAASGFNIKDTYMHWVRQAPGKGLEWVARIDPANGNSKYADSVKGRF
TISADTSKNTAYLQMNSLRAEDTAVYYCAPFGYYVSDYAMAYWGQGTLVTVSSASTKGPSVFPLAPSSKS
TSGGTAALGCLVKDYFPEPVTVSWNSGALTSGVHTFPAVLQSSGLYSLSSVVTVPSSSLGTQTYICNVNH
KPSNTKVDKKVEPKSC

In some embodiments, the therapeutic domain provided herein comprise an ipilimumab-derived fragment that is at least 90%, 91%, 92%, 93%, 94%, 95%, 96%, 97%, 98% or 99% identical to the sequence:

Therapeutic domain of Engineered ipilimumab design 16
(SEQ ID NO: 25)
QVQLVESGGGVVQPGRSLRLSCAASGFTFSSYTMHWVRQAPGKGLEWVTFISYDGNNKYYADSVKGRFT
ISRDNSKNTLYLQMNSLRAEDTAIYYCARTGWLGPFDYWGQGTLVTVSSASTKGPSVFPLAPSSKSTSGG
TAALGCLVKDYFPEPVTVSWNSGALTSGVHTFPAVLQSSGLYSLSSVVTVPSSSLGTQTYICNVNHKPSNT
KVDKRVEPKSC

Suitably, the cleavable domain does not interfere with the binding of the agent to its binding partner. It will be appreciated that the cleavable domain is positioned in the agent such that, in a cleaved state (therapeutic domain) or uncleaved state (agent), the ability of the agent to bind to its binding partner is not affected by the cleavable domain. Those skilled in the art would be capable of selecting an appropriate method to measure the ability of the agent to bind to its binding partner, for example by an ELISA or BIAcore.

In another embodiment the therapeutic domain binds to a binding partner when the cleavable domain has not been cleaved. In other words, it is not necessary for the therapeutic domain to be released from the agent for it to bind to its binding partner. In one embodiment, the therapeutic domain is not masked. By ‘masked’, we include the meaning that the therapeutic domain is blocked from binding to its binding partner. Upon cleavage of a cleavable domain, a masked therapeutic domain would become unmasked. In another embodiment, the agent does not comprise a masking domain.

In a further embodiment, the therapeutic domain retains its ability to bind to its binding partner once the cleavable domain has been cleaved. It will be appreciated that the release of the stability domain from the therapeutic domain does not negatively decrease the binding of the therapeutic domain to its target.

In one embodiment, the therapeutic domain binds its target with a half maximal inhibitory concentration (IC₅₀) of from 0.2 to 1.4 nanomolar (nM) relative to its target, such as from about 0.2, 0.205, 0.210, 0.215, 0.220, 0.225, 0.230, 0.235, 0.240, 0.245, 0.250, 0.255, 0.260, 0.265, 0.270, 0.275, 0.276, 0.277, 0.278, 0.279 or 0.280 nM to about 0.260, 0.265, 0.270, 0.275, 0.280, 0.285, 0.290, 0.295, 0.3, 0.4, 0.5, 0.6, 0.7, 0.8, 0.9, 1, 1.1, 1.2, 1.325, 1.350, 1.375, 1.380, 1.385, 1.39, 1.395 or 1.4. The IC₅₀ indicates the potency of the therapeutic domain in inhibiting a specific biological or biochemical function. It is a quantitative measure that indicates how much of a particular inhibitory substance is needed to inhibit a given biological process or biological component by 50%. Any suitable means can be used to measure IC₅₀ for example, by functional assays or with competition binding assay (such as an enzyme-linked immunosorbent assay (ELISA)).

In one embodiment, the therapeutic domain has a dissociation constant (K_{D}) of from 1.5 to 2.25 nM, such as from about 1.50, 1.51, 1.52, 1.53, 1.54, 1.55, 1.56, 1.57, 1.58, 1.59, 1.60, 1.61, 1.62, 1.63, 1.64, 1.65, 1.66, 1.67, 1.68, 1.69, 1.70, 1.71, 1.72, 1.73, 1.74, 1.75, 1.76, 1.77, 1.78, 1.79, 1.80, 1.81, 1.82, 1.83, 1.84, 1.85, 1.86, 1.87, 1.88, 1.89 or 1.90 to about 1.70, 1.71, 1.72, 1.73, 1.74, 1.75, 1.76, 1.77, 1.78, 1.79, 1.80, 1.81, 1.82, 1.83, 1.84, 1.85, 1.86, 1.87, 1.88, 1.89, 1.90, 1.91, 1.92, 1.93, 1.94, 1.95, 1.96, 1.97, 1.98, 1.99, 2.00, 2.01, 2.02, 2.03, 2.04, 2.05, 2.06, 2.07, 2.08, 2.09, 2.10, 2.11, 2.12, 2.13, 2.14, 2.15, 2.16, 2.17, 2.18, 2.19, 2.20, 2.21, 2.22, 2.23, 2.24 or 2.25. The K_{D} indicates the equilibrium constant that measures the propensity of one binding domain to dissociate from its target i.e., the binding domain of the therapeutic domain to its binding partner (e.g., a tumour antigen). The smaller the dissociation constant, the more tightly bound the binding domain is, or the higher the affinity between binding domain and its target. Any suitable means can be used to measure K_{D} for example, by functional assays, with competition binding assay or with live or real time binding (such as an BIAcore).

In one embodiment, the cleavable domain does not interfere with the thermal stability of the agent. It will be appreciated that the cleavable domain is positioned in the agent such that, in a cleaved state (therapeutic domain) or uncleaved state (agent), the thermal stability of the agent is not significantly affected by the cleavable domain. Those skilled in the art would be capable of selecting an appropriate method to measure thermal stability, for example fluorescence, static light scattering (SLS) and dynamic light scattering (DLS).

In one embodiment, the agent has a melting temperature (Tₘ) of from 65 degrees centigrade (° C.) to 75° C., such as from 65.1, 65.2, 65.3, 65.4, 65.5, 65.6, 65.7, 65.8, 65.9, 66.0, 66.1, 66.2, 66.3, 66.4, 66.5, 66.6, 66.7, 66.8, 66.9, 67.0, 67.1, 67.2, 67.3, 67.4, 67.5, 67.6, 67.7, 67.8, 67.9, 68.0, 68.1, 68.2, 68.3, 68.4, 68.5, 68.6, 68.7, 68.8, 68.9, 69.0, 69.1, 69.2, 69.3, 69.4, 69.5, 69.6, 69.7, 69.8, 69.9 or 70.0° C. to about 68.0, 68.1, 68.2, 68.3, 68.4, 68.5, 68.6, 68.7, 68.8, 68.9, 69.0, 69.1, 69.2, 69.3, 69.4, 69.5, 69.6, 69.7, 69.8, 69.9, 70.0, 70.1, 70.2, 70.3, 70.4, 70.5, 70.6, 70.7, 70.8, 70.9, 71.0, 71.1, 71.2, 71.3, 71.4, 71.5, 71.6, 71.7, 71.8, 71.9, 72.0, 72.1, 72.2, 72.3, 72.4, 72.5, 72.6, 72.7, 72.8, 72.9, 73.0, 73.1, 73.2, 73.3, 73.4, 73.5, 73.6, 73.7, 73.8, 73.9, 74.0, 74.1, 74.2, 74.3, 74.4, 74.5, 74.6, 74.7, 74.8, 74.9, 75.0° C. The Tₘ of a protein relates to the result of denaturation of the protein. Methods of measuring melting temperatures are well known in the art and include differential scanning calorimetry (DSC), differential scanning fluorometry (DSF) and other well-known thermal shift assays.

In one embodiment, the agent has an aggregation temperature (T_{agg}) of from 65° C. to 80° C., such as from 65.1, 65.2, 65.3, 65.4, 65.5, 65.6, 65.7, 65.8, 65.9, 66.0, 66.1, 66.2, 66.3, 66.4, 66.5, 66.6, 66.7, 66.8, 66.9, 67.0, 67.1, 67.2, 67.3, 67.4, 67.5, 67.6, 67.7, 67.8, 67.9, 68.0, 68.1, 68.2, 68.3, 68.4, 68.5, 68.6, 68.7, 68.8, 68.9, 69.0, 69.1, 69.2, 69.3, 69.4, 69.5, 69.6, 69.7, 69.8, 69.9, 70.0, 70.1, 70.2, 70.3, 70.4, 70.5, 70.6, 70.7, 70.8, 70.9, 71.0, 71.1, 71.2, 71.3, 71.4, 71.5, 72.0, 72.5, 73.0, 73.5, 74.0, 74.5 or 75.0, 75.5, 76.0, 76.5 or 77.0° C. to about 73.0, 73.5, 74.0, 74.5, 75.0, 75.5, 76.0, 76.5, 77.0, 77.5, 78.0, 78.5, 79.0, 79.1, 79.2, 79.3, 79.4, 79.5, 79.6, 79.7, 79.8, 79.9 or 80.0° C. By “T_{agg}”, we include the meaning of the temperature at which the onset of aggregation occurs or the temperature at which molecules have a tendency to aggregate together. Methods of measuring thermal stability are well known in the art and include dynamic light scattering (DLS), static light scattering (SLS) and/or fluorescence.

DLS measures the hydrodynamic size and size distribution of particles in solution and can be plotted over time and temperature. In general, at low temperatures a protein may be stable and show repeatable size (and scattering intensity) measurements, whereas typically at more elevated temperatures (T_{agg}), protein molecules will show a tendency to aggregate.

In one embodiment, the cleavable domain does not interfere with the binding of the stabilisation domain to Fc gamma receptors (FcRs), optionally wherein the Fc gamma receptors is Fc gamma receptor Ia and/or Fc gamma receptor IIa. FcRs are membrane proteins expressed by several hematopoietic cells that recognise the Fc region of several immunoglobulin classes and subclasses. The Fc region of an antibody can bind to Fc receptors (FcγRI, FcγRII, FcγRIII) expressed on the surface of immune cells, complement (C1q) and FcRn (neonatal FcR) in the blood, thereby activating the immune system. The interaction mediated by the antibody Fc domain can strongly influence the functional outcome of antibody therapy including antibody-dependent cell-mediated cytotoxicity (ADCC), antibody-dependent cell-mediated phagocytosis (ADCP) and complement-dependent cytotoxicity (CDC) through the interaction of the Fc domain with Fc receptors on different cell types. Of note, the combination of IgG and neonatal FcR (FcRn) can protect antibodies from being degraded, thereby prolonging its half-life.

In another embodiment, the cleavable domain may interfere with the binding of the stabilisation domain to Fc gamma receptors.

It will be appreciated that the cleavable domain is positioned in the agent such that, in a cleaved state (stability domain) or uncleaved state (agent), the binding of Fc gamma receptors is not significantly affected by the cleavable domain. Those skilled in the art would be capable of selecting an appropriate method to measure binding, for example by functional assays or with competition binding assay (such as an enzyme-linked immunosorbent assay (ELISA)).

In one embodiment, the therapeutic domain reduces or inhibits the proliferation of unwanted cells or the growth of a tumour.

In one embodiment, the therapeutic domain stimulates immune cells, for example immune checkpoint inhibitors (ICIs), immunomodulators, cytokines.

It will be appreciated that an immune cell may comprise any of the following immune cell: lymphocytes (B and T cells), antigen presenting cells (APC), natural killer (NK) cells, macrophages, monocytes, dendritic cells. T cells recognise peptide antigens, derived from proteins degraded intracellularly, that are loaded onto cell surface MHC molecules, a process called antigen presentation (APC).

By “stimulation”, we include the meaning that an immune cell, such as a T cell, is activated and proliferates. In some embodiments, stimulation also includes co-stimulation, preventing recruitment of inhibitory effectors and preventing T cell exhaustion.

In another embodiment, the therapeutic domain blocks immune cells. The terms “blocks” and “inhibits” are used interchangeably and encompass both partial and complete inhibition/blocking. In one embodiment the blocked immune cells are regulatory T cells (Tregs). Tregs are a specialised subpopulation of T cells that play a critical role in preventing autoimmunity, by inhibiting T cell proliferation and cytokine production.

As discussed herein, the primary function of the therapeutic domain is to exert a clinical or therapeutically beneficial effect on the target tissue. Optionally, the therapeutic domain may have a secondary function wherein the therapeutic domain targeting the agent to the target tissue. For example, wherein the therapeutic domain according to the claimed invention is an antibody drug conjugate (ADC), it will comprise a Fab domain (which targets the ADC to the unwanted cells), and a cytotoxic drug (also known as the payload) chemically linked to a Fab region. Therefore, the therapeutic domain may function to (i) exert a clinical or therapeutically beneficial effect and (ii) target itself to the target tissue.

### Stabilisation Domain

In some embodiments, the stabilisation domain is a protein-based domain or a polymer. For example, the protein-based domain may comprise a structured polypeptide (e.g. an IgG Fc region, HSA), an elastin-like peptide (ELPylationn), an inert polypeptide, e.g., XTEN (also known as recombinant PEG or “rPEG”), a homoamino acid polymer (HAP; HAPylation) proline-alanine-serine polymer (PAS; PASylation). In another embodiment, the stabilisation domain is not a protein, such as a chemical moiety (e.g. PEGylation or hyaluronic acid).

In one embodiment, the stabilisation domain targets the therapeutic domain to unwanted cells. By “stabilisation domain targeting the therapeutic domain”, we include the meaning that that stabilisation domain may by a specific binding partner of an entity expressed by or associated with unwanted cells. In another embodiment, the stabilisation domain does not target the therapeutic domain to the unwanted cells.

In some embodiments, the stabilisation domain is: i. a Fc region, optionally wherein the Fc region is an IgG, IgE, IgM, IgD or IgA family Fc region or a bispecific Fc region; or; or ii. a PEGylated domain; or iii. a PASylation domain; or iv. a XTENylated domain; or v. a HESylated domain; or v. a lipidated domain; or vii. a glycosylated domain; or viii. the Human Serum Albumin (HSA) protein or fragment thereof; or ix. a HSA binding protein; or x. a protein binding to a blood circulating cell or xi. a Fab domain, optionally wherein the Fab domain is directed HSA or directed to the same antigen as the antigen-binding domain. Preferably, the stabilisation domain is: i. a Fc region, optionally wherein the Fc region is an IgG, IgE, IgM, IgD or IgA family Fc region or a bispecific Fc region. In one embodiment, the stabilisation domain does not comprise a Fab domain.

The Fc region and/or the hinge region may be selected from any type of immunoglobulins which includes IgM, IgG, IgA, IgD, IgE. Each isotype also includes different subtypes. For example, subtypes of IgG include IgG1, IgG2, IgG3, IgG4 and subtypes of IgA include IgA1 and IgA2. It will be appreciated that there are different isotypes and subtypes in different species.

Other serum proteins that may act as stabilisation domain may include albumin, fibrinogen, fibronectin, haemoglobin, transferrin, an immunoglobulin domain.

Suitable stabilisation domain may display one or more N-glycosylation motifs. N-glycosylation the process of attachment of a glycan oligosaccharide to an amide nitrogen of an asparagine (N) residue of a protein. N-glycosylation motifs can be found in the YNSTY (SEQ ID NO: 26) sequence of the CH2 of the Fc region, for example N297.

The Fc region contains the constant regions (CH) CH2, and CH3 from the heavy chains. The general shape of an antibody is a Y, with a flexible hinge (interdomain) region at the centre of the Y. The flexibility of the interdomain hinge region is important for the bivalent binding of an antibody, allowing the two binding pockets to interact with antigenic sites at variable distances.

The Fc region contains the constant regions (CH) CH2, and CH3 from the heavy chains. The Fc region may be composed of homo-immunoglobulin molecules, preferably homo-IgG molecules. In one embodiment, the stabilisation domain is not a hetero-IgG molecule.

In some embodiments, the stabilisation domain comprises a sequence that is at least 90%, 91%, 92%, 93%, 94%, 95%, 96%, 97%, 98% or 99% identical to sequences selected from the group consisting of:

Stabilisation domain of Engineered trastuzumab designs 1-6, D9, D12, D13,
D15 and D18-
(SEQ ID NO: 27)
ELLGGPSVFLFPPKPKDTLMISRTPEVTCVVVDVSHEDPEVKFNWYVDGVEVHNAKTKPREEQYNSTYRVV
SVLTVLHQDWLNGKEYKCKVSNKALPAPIEKTISKAKGQPREPQVYTLPPSREEMTKNQVSLTCLVKGFYP
SDIAVEWESNGQPENNYKTTPPVLDSDGSFFLYSKLTVDKSRWQQGNVFSCSVMHEALHNHYTQKSLSL
SPGK
Stabilisation domain of Engineered trastuzumab design 7
(SEQ ID NO: 28)
FLFPPKPKDTLMISRTPEVTCVVVDVSHEDPEVKFNWYVDGVEVHNAKTKPREEQYNSTYRVVSVLTVLH
QDWLNGKEYKCKVSNKALPAPIEKTISKAKGQPREPQVYTLPPSREEMTKNQVSLTCLVKGFYPSDIAVEW
ESNGQPENNYKTTPPVLDSDGSFFLYSKLTVDKSRWQQGNVFSCSVMHEALHNHYTQKSLSLSPGK
Stabilisation domain of Engineered pembrolizumab designs D14a and D14b
(SEQ ID NO: 29)
EFLGGPSVFLFPPKPKDTLMISRTPEVTCVVVDVSQEDPEVQFNWYVDGVEVHNAKTKPREEQFNSTYRV
VSVLTVLHQDWLNGKEYKCKVSNKGLPSSIEKTISKAKGQPREPQVYTLPPSQEEMTKNQVSLTCLVKGF
YPSDIAVEWESNGQPENNYKTTPPVLDSDGSFFLYSRLTVDKSRWQEGNVFSCSVMHEALHNHYTQKSL
SLSLGK

As discussed herein, the primary function of the stabilisation domain is to stabilise or to extend the half-life of the agent in a biological system. Optionally, the stabilisation domain may have a secondary function wherein the stabilisation domain has a clinical or therapeutically beneficial effect. For example, wherein the agent according to the claimed invention is an antibody drug conjugate (ADC), it will comprise a therapeutic domain (which targets the ADC to the unwanted cells), a cleavable domain and a stabilisation domain. The stabilisation domain may comprise a cytotoxic drug (also known as the payload) chemically linked to an Fc region. Therefore, the stabilisation may function to (i) stabilise or increase the half-life of the agent and (ii) exert a clinical or therapeutically beneficial effect.

### Cleavable Domain

The cleavable domain is positioned between the therapeutic domain and the stabilisation domain and connects the therapeutic and stabilisation domains in the agent. Selective cleavage of the cleavable domain releases the therapeutic domain from the stabilisation domain enabling the therapeutic domain to carry out its therapeutic function. Typically, the stabilisation domain will then be degraded or released and cleared by GFB. It will be appreciated that when the agent is cleaved, part of the cleavable domain may remain attached to the therapeutic domain, and part of the cleavable domain may remain attached to the stabilisation domain.

The cleavable domain comprises at least one cleavage site. “Cleavage site” refers to a site of an amino acid sequence that is a substrate for an enzyme, such as an extracellular enzyme. For example, the cleavage site for the enzyme u plasminogen activator (uPA), fibroblast activation protein (FAP), legumain or MT-SP1. The cleavage site may be one that is cleavable by an enzyme such as any of a protease, a nuclease, a lipase, a lyase, a phosphatase or a carbohydrase, which may or may not be membrane-bound. By “protease”, we include the meaning of an enzyme that catalyses proteolysis (i.e. the breaking down of a protein into smaller polypeptides or single amino acids). In one embodiment, the cleavage site is not the amino acid sequence DEVD.

It will be appreciated that the cleavable domain may or may not comprise a hinge region or part thereof. For example, the cleavable domain may comprise a cleavage site and linker sequences which connect the therapeutic domain (such as OcoMYC) to a stabilisation domain, wherein the stabilisation domain is not an Fc region or part thereof (such as HSA).

In some embodiments, the cleavage site is located in a hinge region. By “a hinge region”, we include the meaning of a hydrophilic sequence of the heavy chains of an antibody. In general, the hinge is responsible for linking a Fab region to an Fc region in a flexible manner. The hinge can be divided into three parts: the upper hinge, middle hinge, and lower hinge. The middle hinge is where the two heavy chains meet, and this is the part that holds the antibody together. In some embodiments, the cleavable domain is located in the upper hinge, middle hinge, or lower hinge, preferably in the upper hinge or in the lower hinge. Preferably, the cleavable domain is located in the upper hinge. Advantages of the cleavable domain in the hinge region include providing the ability of fully separating the therapeutic and stabilisation domains after cleavage.

In some embodiments, the cleavable domain comprises a sequence that is at least 90%, 91%, 92%, 93%, 94%, 95%, 96%, 97%, 98% or 99% identical to sequences selected from the group consisting of:

Cleavable domain of Engineered trastuzumab design 1
(SEQ ID NO: 30)
GSLSGRSDNHDKTHTCPPCPAP
Cleavable domain of Engineered trastuzumab design 2
(SEQ ID NO: 31)
LSGRSDNHDKTHTCPPCPAP
Cleavable domain of Engineered trastuzumab design 3
(SEQ ID NO: 32)
GSLSGRSDNHGSDKTHTCPPCPAP
Cleavable domain of Engineered trastuzumab design 4
(SEQ ID NO: 33)
LSGRSDNHCPPCPAP
Cleavable domain of Engineered trastuzumab design 5
(SEQ ID NO: 34)
LSGRSDNHTCPPCPAP
Cleavable domain of Engineered trastuzumab designs 6 and 7
(SEQ ID NO: 35)
DKTHTCPPCPAPLSGRSDNH
Cleavable domain of Engineered trastuzumab design 9
(SEQ ID NO: 36)
GSPRAEALKGGGSASDKTHTCPPCPAP
Cleavable domain of Engineered designs 12, 13, 15 and 16
(SEQ ID NO: 37)
GSLSGRSDNHGSASDKTHTCPPCPAP
Cleavable domain of Engineered pembrolizumab design 14a
(SEQ ID NO: 38)
GSLSGRSDNHGSGPPCPPCPAP
Cleavable domain of Engineered pembrolizumab design 14b
(SEQ ID NO: 39)
GPPGSLSGRSDNHGSCPPCPAP
Cleavable domain of Engineered trastuzumab design 17
(SEQ ID NO: 40)
LSGRSDNHGSASDKTHTCPPCPAP
Cleavable domain of Engineered trastuzumab design 18
(SEQ ID NO: 41)
GGGSGGGSLSGRSDNHGSASDKTHTCPPCPAP

In one embodiment, the protease that acts on the cleavage site is expressed by and/or accumulates in the vicinity of unwanted cells or a tumour. Preferably, the protease is a tumour-specific protease. Thus, when the unwanted cells are tumour cells, the cleavage site may be cleavable selectively by proteases that are found in the vicinity of the tumour cells.

Specificity is increased by virtue of the cleavage site only being cleaved in the vicinity of the unwanted cells. For example, tumour cells secrete proteases that are required by tumours for invasion of local tissues and metastasis, and so by including a tumour-specific protease cleavage site in the agent, the specificity of the agent for the tumour is increased.

“Vicinity” or “tumour microenvironment” herein refers to the area at and/or near to the surface of the cells, such as the environment that immediately surrounds the cells e.g. blood vessels, immune cells, fibroblasts, signalling molecules and/or extracellular matrix (ECM), blood, lymph, and other body fluids. In one embodiment, the vicinity starts at the tumour site and extends at least 10 micrometres (μm), at least 20 μm, at least 30 μm, at least 40 μm, at least 50 μm, at least 60 μm, at least 70 μm, at least 80 μm, at least 90 μm, at least 100 μm, at least 120 μm, at least 140 μm, at least 160 μm, at least 180 μm, at least 200 μm away from the tumour.

In some embodiments, the concentration of enzymes may be low in some tissue where the invention does not cleave and does not penetrate deeper, for example in non-disease tissue. In some embodiments, the enzymes are not active or is significantly less active in healthy (e.g. non-diseased) tissue or tissues not intended for therapy.

It is also appreciated that since the cleavage site in the agent confers specificity on where the therapeutic domain is released, binding of therapeutic domain to non-disease cells, in the vicinity of which the cleavage site is not cleaved, may also be tolerated.

The proteases may include any of a cysteine protease (including the Cathepsin family B, L, S etc), an aspartyl or aspartic protease (including Cathepsin D and E, or Napsin A) and a serine protease (including Cathepsin A and G, Thrombin, Plasmin, uPA, tissue Plasminogen Activator (tPA), matriptase, MT-SP1, fibroblast activation protein (FAP)).

The protease may be a metalloproteinase (also known as a metallopeptidase or metalloproteinase) (MMP1-28) including both membrane-bound (MMP14-17 and MMP24-25) and secreted forms (MMP1-13 and MMP18-23 and MMP26-28). The protease may belong to the A Disintegrin and Metalloproteinase (ADAM) and A Disintegrin, or Metalloproteinase with Thrombospondin Motifs (ADAMTS) families of proteases. Other examples include CD10 (CALLA), prostate specific antigen (PSA) and coagulation factors. It is appreciated that the proteases may or may not be membrane-bound. The proteases may include any of them in combination thereof.

In order to treat a particular tumour type, the skilled person will typically select a protease cleavage site that is selectively cleaved by a protease known to be highly expressed in that tumour type. For example, to treat breast cancer, it is preferred to use a protease cleavage site cleavable by any of uPA, Napsin A, tPA, legumain, matriptase, matriptase 2, Cathepsin K, Cathepsin 0, MMP1, MMP2, MMP3, MMP11, MMP12, MMP17, ADAM9, ADAM10, ADAM12, ADAM15, ADAM17, ADAM28 or ADAMTS15, and so on.

In some embodiments, the cleavable domain comprises one or more linker domains present between the therapeutic domain and the stabilisation domain.

Such linker domains can allow for greater accessibility of the enzyme and be selected from the group consisting of amino acid sequences represented by (GₘSₙ)ₓ or (GGNGT)ₓ (SEQ ID NO: 42) or (YGNGT)ₓ (SEQ ID NO: 43) wherein m and n are each independently selected from the group consisting of integers from 1 to 8 (e.g., 1, 2, 3, 4, 5, 6, 7 or 8), and x is independently selected from the group consisting of integers from 1 to 20 (e.g., 1, 2, 3, 4, 5, 6, 7, 8, 9, 10, 11, 12, 13, 14, 15, 16, 17, 18, 19 or 20). In some specific embodiments, the linker has an amino acid sequence represented by (G₄S)ₓ (SEQ ID NO: 44), wherein x is independently selected from the group consisting of integers from 1 to 6 (e.g., x is 4 or 5) such as (G₄S)₂ (SEQ ID NO: 45). The linker may also be another linker having similar flexibility and length to the amino acid sequence represented by (GₘSₙ)ₓ or (GGNGT)ₓ (SEQ ID NO: 42) or (YGNGT)ₓ (SEQ ID NO: 43) described above; the linker may also be selected from the group consisting of GPPGS (SEQ ID NO: 46), GSGPP (SEQ ID NO: 47), AKTTPKLEEGEFSEAR (SEQ ID NO: 48), AKTTPKLEEGEFSEARV (SEQ ID NO: 49), AKTTPKLGG (SEQ ID NO: 50), SAKTTPKLGG (SEQ ID NO: 51), AKTTPKLEEGEFSEARV (SEQ ID NO: 52), SAKTTP (SEQ ID NO: 53), SAKTTPKLGG (SEQ ID NO: 54), RADAAP (SEQ ID NO: 55), RADAAPTVS (SEQ ID NO: 56), RADAAAAGGPGS (SEQ ID NO: 57), RADAAAA(G₄S)₄ (SEQ ID NO: 58), SAKTTP (SEQ ID NO: 59), SAKTTPKLGG (SEQ ID NO: 60), SAKTTPKLEEGEFSEARV (SEQ ID NO: 61), ADAAP (SEQ ID NO: 62), ADAAPTVSIFPP (SEQ ID NO: 63), TVAAP (SEQ ID NO: 64), TVAAPSVFIFPP (SEQ ID NO: 65), QPKAAP (SEQ ID NO: 66), QPKAAPSVTLFPP (SEQ ID NO: 67), AKTTPP (SEQ ID NO: 68), AKTTPPSVTPLAP (SEQ ID NO: 69), AKTTAP (SEQ ID NO: 70), AKTTAPSVYPLAP (SEQ ID NO: 71), ASTKGP (SEQ ID NO: 72), ASTKGPSVFPLAP (SEQ ID NO: 73), GENKVEYAPALMALS (SEQ ID NO: 74), GPAKELTPLKEAKVS (SEQ ID NO: 75) and GHEAAAVMQVQYPAS (SEQ ID NO: 76).

In some embodiments, the cleavable domain overlaps with the therapeutic domain and/or the stabilisation domain.

In one embodiment, the cleavable domain may be an additional sequence (which adds further amino acid to the biologic). In another embodiment, the cleavable domain is generated by creating one or more mutations (e.g. addition, substitution or deletion) to the endogenous sequence (which does not add any further amino acids to the biologic). In yet another embodiment, it could be a combination of the addition of a sequence and mutagenesis.

In some embodiments, the cleavable domain overlaps with at least 2%, at least 5%, at least 10%, at least 15%, at least 20%, at least 25%, at least 30%, at least 35%, at least 40%, at least 50%, at least 60%, at least 70%, at least 80%, at least 90%, at least 99%, at least 100% with the therapeutic domain and/or stabilisation domain.

Typically, small cleavage sites (including any linkers if present) are favourable because the risk of immunogenicity (to foreign/non-host sequences) is lower.

In some embodiments, the at least one cleavage site is 4 to 20 amino acids in length. For example, the at least one cleavage site has a length of 4 to 6 amino acids, 4 to 8 amino acids, 4 to 10 amino acids, 4 to 12 amino acids, 4 to 14 amino acids, 4 to 16 amino acids, 4 to 18 amino acids, 6 to 8 amino acids, 6 to 10 amino acids, 6 to 12 amino acids, 6 to 14 amino acids, 6 to 16 amino acids, 6 to 18 amino acids, 6 to 20 amino acids, 8 to 10 amino acids, 8 to 12 amino acids, 8 to 14 amino acids, 8 to 16 amino acids, 8 to 18 amino acids, 8 to 20 amino acids, 10 to 12 amino acids, 10 to 14 amino acids, 10 to 16 amino acids, 10 to 18 amino acids, 10 to 20 amino acids, 12 to 14 amino acids, 12 to 16 amino acids, 12 to 18 amino acids, 12 to 20 amino acids, 14 to 16 amino acids, 14 to 18 amino acids, 14 to 20 amino acids, 16 to 18 amino acids, 16 to 20 amino acids, 18 to 20 amino acids.

In a further embodiment, the agent is a protein or polypeptide, the stabilisation or the therapeutic domain comprises a Fc region and a hinge region and the cleavable domain is located: i. N-terminal to the hinge region and after the therapeutic domain; or ii. C-terminal to the hinge region and before the CH2 domain of the Fc region.

In some embodiments, the uncleaved agent has a structural arrangement from N-terminus to C-terminus as follows: i. Therapeutic domain-cleavable domain-Hinge region-Fc region; or ii. Therapeutic domain-hinge region-cleavable domain-Fc region.

The cleavable domain may or may not occur either before or either after the hinge region. This difference in location will give two separate moieties that may yield a monovalent or multivalent (such a bivalent) binding protein. The engineering design will depend on the preferred mode of action for the therapeutic.

In some embodiments, the cleavable domain is not cleavable by a tumour-specific protease from the list consisting of Gelatinase A (MMP-2), Stromelysin 1 (MMP-3), Matrilysin (MMP-7), Gelatinase B (MMP-9), Macrophage metalloelastase (MMP-12), Collagenase-3 (MMP-13), Cathepsin G9). In another embodiment, the cleavable domain is not cleavable by the protease Capsase-3 or by the PreScission protease. By “PreScission protease”, we include the meaning of a fusion of GST and a human rhinovirus protease.

In a preferred embodiment, the therapeutic domain is a Fab, the cleavable domain comprises a uPA cleavage site, and the stabilisation domain is an Fc region.

In a preferred embodiment, the therapeutic domain is a Fab, the cleavable domain comprises a ADAM10 cleavage site, and the stabilisation domain is an Fc region.

In another preferred embodiment, the therapeutic domain is a VHH, the cleavable domain comprises a uPA cleavage site, and the stabilisation domain is an Fc region.

In a preferred embodiment, the therapeutic domain is a ScFv, the cleavable domain comprises a uPA cleavage site, and the stabilisation domain is an Fc region.

### Therapeutic Uses

The agent is suitable for use as a medicament, such as in therapy, such as in the treatment or prevention of a tumour, optionally of a cancer.

By ‘treatment’ we include both therapeutic and prophylactic treatment of the patient. The term ‘prophylactic’ is used to encompass the use of an agent, or formulation thereof, as described herein which either prevents or reduces the likelihood of cancer, or the spread, dissemination, or metastasis of localised cancer in a patient or subject. The term ‘prophylactic’ also encompasses the use of an agent, or formulation thereof, as described herein to prevent recurrence of cancer in a patient who has previously been treated for cancer.

As used herein, the terms “treatment” or “treating” denote an approach for obtaining a beneficial or desired result including and preferably a beneficial or desired clinical result. Such beneficial or desired clinical results include, but are not limited to, one or more of the following: reducing the proliferation of (or destroying) cancerous cells or other diseased, reducing metastasis of cancerous cells found in cancers, shrinking the size of the tumour, decreasing symptoms resulting from the disease, increasing the quality of life of those suffering from the disease, decreasing the dose of other medications required to treat the disease, delaying the progression of the disease, and/or prolonging survival of individuals.

Upon binding, the agent may directly exert a therapeutic effect (e.g. inducing cell death via Antibody Dependent Cellular Cytotoxicity (ADCC), complement dependent cytotoxicity (CDC) or by virtue of carrying a radioisotope or other cytotoxic moiety). Alternatively, the bound agent may serve as a diagnostic (imaging) tool and may guide the choice of therapy or aid surgical removal of the unwanted cells.

In a particularly preferred embodiment, the cleavage site is selectively cleaved outside of the unwanted cell, at or near its surface, so that the therapeutic domain is released on without needing to be internalised.

Suitably the vicinity of unwanted cells or the tumour comprises a high titre and/or a high activity of tumour-specific proteases.

The titre and/or activity of a protease in the vicinity of unwanted cells or the tumour will be considered “high” if the titre and/or activity of the protease is significantly higher than titre and/or activity of the protease at other locations considered further than the vicinity of unwanted cells of the tumour. Those skilled in the art would be capable of selecting an appropriate assay to measure titre and activity of the protease, for example ELISA, activity-based protein profiling (ABPP), mass spectrometry, fluorescent probe imaging.

Suitably, the subject of treatment or prevention is a mammal, preferably a human. In some embodiments, the subject is a non-human mammal, such as a rodent, a non-human primate, companion animal (e.g. cat, dog, horse), farm animal, work animal, zoo animal.

Suitably, the tumour is a solid tumour. In some embodiments, the cancer is selected from the group consisting of: Adenocarcinoma, Adenosarcoma, Adrenal cancer, Adrenocortical carcinoma, Anal cancer, Anaplastic astrocytoma, Angiosarcoma, Appendix cancer, Astrocytoma, Basal cell carcinoma, B-Cell lymphoma, Bile duct cancer, Bladder cancer, Bone cancer, Bowel cancer, Brain cancer, Brain stem glioma, Brain tumour, Breast cancer, Carcinoid tumours, Cervical cancer, Cholangiocarcinoma, Chondrosarcoma, Colon cancer, Colorectal cancer, Craniopharyngioma, Cutaneous melanoma, Diffuse astrocytoma, Ductal carcinoma in situ, Endometrial cancer, Ependymoma, Epithelioid sarcoma, Esophageal cancer, Ewing sarcoma, Extrahepatic bile duct cancer, Eye cancer, Fallopian tube cancer, Fibrosarcoma, Gallbladder cancer, Gastric cancer, Gastrointestinal cancer, Gastrointestinal carcinoid cancer, Gastrointestinal stromal tumours, General, Germ cell tumour, Glioblastoma multiforme, Glioma, Head and neck cancer, Hemangioendothelioma, Hodgkin's disease Hypopharyngeal cancer, Infiltrating ductal carcinoma, Infiltrating lobular carcinoma, Inflammatory breast cancer, Intestinal Cancer, Intrahepatic bile duct cancer, Invasive/infiltrating breast cancer, Islet cell cancer, Jaw cancer, Kaposi sarcoma, Kidney cancer, Laryngeal cancer, Leiomyosarcoma, Leptomeningeal metastases Lip cancer, Liposarcoma, Liver cancer, Lobular carcinoma in situ, Low-grade astrocytoma, Lung cancer, Lymph node cancer, Lymphoma, Male breast cancer, Medullary carcinoma, Medulloblastoma, Melanoma, Meningioma, Merkel cell carcinoma, Mesenchymal chondrosarcoma, Mesenchymous, Mesothelioma, Metastatic breast cancer, Metastatic melanoma, Metastatic squamous neck cancer, Mixed gliomas, Mouth cancer, Mucinous carcinoma, Mucosal melanoma, Multiple myeloma, Nasal cavity cancer, Nasopharyngeal cancer, Neck cancer, Neuroblastoma, Neuroendocrine tumours, Non-small cell lung cancer, Oat cell cancer, Ocular cancer, Ocular melanoma, Oligodendroglioma, Oral cancer, Oral cavity cancer, Oropharyngeal cancer, Osteogenic sarcoma, Osteosarcoma, Ovarian cancer, Ovarian epithelial cancer, Ovarian germ cell tumour, Ovarian primary peritoneal carcinoma, Ovarian sex cord stromal tumour, Paget's disease, Pancreatic cancer, Papillary carcinoma, Paranasal sinus cancer, Parathyroid cancer, Pelvic cancer, Penile cancer, Peripheral nerve cancer, Peritoneal cancer, Pharyngeal cancer, Pheochromocytoma, Pilocytic astrocytoma, Pineal region tumour, Pineoblastoma, Pituitary gland cancer, Primary central nervous system lymphoma, Prostate cancer, Rectal cancer, Renal cell cancer, Renal pelvis cancer, Rhabdomyosarcoma, Salivary gland cancer, Sarcoma, Sarcoma, bone, Sarcoma, soft tissue, Sarcoma, uterine, Sinus cancer, Skin cancer, Small cell lung cancer, Small intestine cancer, Soft tissue sarcoma, Spinal cancer, Spinal column cancer, Spinal cord cancer, Spinal tumour, Squamous cell carcinoma, Stomach cancer, Synovial sarcoma, T-cell lymphoma, Testicular cancer, Throat cancer, Thymoma/thymic carcinoma, Thyroid cancer, Tongue cancer, Tonsil cancer, Transitional cell cancer, Transitional cell cancer, Transitional cell cancer, Triple-negative breast cancer, Tubal cancer, Tubular carcinoma, Ureteral cancer, Ureteral cancer, Urethral cancer, Uterine adenocarcinoma, Uterine cancer, Uterine sarcoma, Vaginal cancer, and Vulvar cancer.

In a further aspect, there is a method for the treatment or prevention of a tumour, optionally of cancer, wherein the method comprises one or more of the following steps: a) obtaining a sample of the tumour and/or the vicinity of the tumour; b) determining one or more proteases expressed by the tumour and/or present in the vicinity of the tumour; c) administering to the subject an agent wherein the cleavable domain is cleavable by the one or more proteases determined to be expressed by said tumour and/or present in said vicinity of the tumour.

The term “sample” includes any biological sample from the individual, to be tested in the methods and uses of the invention. It will be appreciated that the sample may comprise one or more tissue, cell and/or biological fluid taken from (such as isolated from) the individual (e.g., blood; serum; plasma; serum plasma; urine; saliva; intestinal cells; biopsy; stool).

By “determining one or more proteases expressed by the tumour and/or present in the vicinity of the tumour”, we include the meaning of determining whether or not the sample contains one or more proteases expressed by the tumour and/or present in the vicinity of the tumour. Preferably, this comprises exposing the agent to the sample and determining which proteases are expressed and/or present.

In yet a further aspect, the invention provides a method of treating or preventing cancer, wherein the method comprises administering one or more agents according to the invention.

Preferably, the one or more agents are administered by injection or infusion. However, in practice it can be administered by any suitable means. Those skilled in the art would be capable of selecting an appropriate route of administration.

In another aspect, the invention provides a pharmaceutical composition, comprising an agent according to the invention, and optionally a pharmaceutically acceptable carrier, diluent or excipient.

The pharmaceutical composition in accordance with the invention may be administered with suitable pharmaceutically acceptable carriers, excipients, and other agents that are incorporated into formulations to provide improved transfer, delivery, tolerance, and the like. By “pharmaceutically acceptable” we include that the formulation is sterile and pyrogen free. Suitable pharmaceutically acceptable carriers, excipients or diluents are well known in the art of pharmacy. The pharmaceutically acceptable carriers, excipients or diluents must be “acceptable” in the sense of being compatible with the agent of the invention and not deleterious to the recipients thereof. Typically, the pharmaceutically acceptable carriers, excipients or diluents will be water or saline which will be sterile and pyrogen free; however, other pharmaceutically acceptable carriers, excipients or diluents may be used.

In yet another aspect, the invention provides the agent according to the invention for use in preventing or treating a condition characterised by the presence of unwanted cells, optionally wherein the condition is cancer (i.e. the unwanted cells are tumour cells).

By a “condition characterised by the presence of unwanted cells” we include any biological or medical condition or disorder in which at least part of the pathology is mediated by the presence of unwanted cells. The condition may be caused by the presence of the unwanted cells or else the presence of the unwanted cells may be an effect of the condition. Examples of particular conditions include tumours (benign or malignant), autoimmune conditions, cardiovascular diseases, degenerative diseases, diabetes, allergic disease (e.g. asthma), neurodegenerative diseases such as Alzheimer's, transplantation patients and infectious diseases. It will be appreciated that the agent also has utility in regenerative medicine (e.g. laboratory grown organs or tissues). It is particularly preferred if the condition is a tumour (e.g. a malignant disease) and the unwanted cells are tumour cells or tumour-associated tissue.

In another aspect, the invention provides a method of improving the penetrability of a therapeutic domain into a tissue or tumour, wherein the method comprises engineering an agent comprising: i) a therapeutic domain; ii) a cleavable domain; and iii) a stabilisation domain.

By “engineering an agent”, we include the meaning of genetic modification. Such genetic modification could include (i) mutations of nucleotide bases of the agent (such as one or more addition, one or more deletion, one or more substitution, or a combination thereof); and (ii) expression (such as recombinant expression) in a cell culture system (such as mammalian cells, bacteria, yeast or insect cells).

In yet another aspect, the invention provides use of the agent according to the invention in the manufacture of a medicament for the treatment or prevention of a tumour, optionally for the treatment or prevention of cancer.

A subject may be treated with a single dose, or multiple doses, of an effective amount of the agents or pharmaceutical compositions of the invention. Where multiple administrations are made, these may be made at a rate of, for example, once, twice, three times, four times or more often per day, week or month, and may be continued for a period of time necessary and effective obtain a therapeutically or prophylactically beneficial effect. For example, treatment may continue for 1, 2, 3, 4, 5, 6, 7, 8, 9, 10, 11, 12 or more days, weeks, months or years, or even for the rest of the life of the subject. The amount of the agent which is administered to the individual is an amount effective to combat the particular individual's condition.

In some embodiments, the agent is administered in combination with one or more additional agents, such as a chemotherapeutic agent, an immunotherapeutic agent or a radiotherapeutic agent. In some embodiments, the agents of the invention and the additional agent(s) are formulated in a single composition. In an alternative embodiment, the agent of the invention and the additional agent(s) are administered as two or more separate compositions. In yet another embodiment, the agent of the invention and the additional agent(s) are administered simultaneously. In an alternative embodiment, the agent of the invention and the additional agent(s) are administered sequentially.

It may be appropriate to administer a particular protease inhibitor so as to improve the target selectivity of the agent of the invention. For example, if a therapeutic domain is known to bind cells in both the heart and breast tissue, but only those in the breast are to be targeted, it may be desirable to administer an additional agent that selectively inhibits the protease in the heart but not the breast.

In other words, an additional agent is administered to inhibit a protease that resides in the vicinity of wanted cells but not in the vicinity of unwanted cells. This is particularly useful in the event that a cleavable domain of the agent of the invention is cleavable by multiple enzymes, some of which reside in the vicinity of unwanted cells and some of which reside in the vicinity of wanted cells. In this case, targeting specificity may be improved by administering a protease inhibitor that inhibits a protease that resides in the vicinity of wanted cells but nevertheless is capable of cleaving the cleavable domain and therefore releasing the therapeutic domain of the agent of the invention. The effect of administering the inhibitor would be to ensure that the therapeutic domain is preferentially released in the vicinity of the unwanted cells.

In yet another aspect, the invention provides a method of improving the efficacy of an agent, the method comprising engineering the agent to comprise: i) a therapeutic domain; ii) a cleavable domain; and iii) a stabilisation domain wherein the cleavable domain is positioned between the therapeutic domain and the stabilising domain. Any feature described above may be used in this method and/or agent.

Small agents offer advantageous pharmacokinetic properties (such as a better ability to penetrate solid tumours, higher tumour uptake levels, improved specificity and/or efficacy, more predictable, more linear elimination). However, small agents also carry significant disadvantages such as an ability to be rapidly eliminated (via the GFB for example), short half-lives etc.

To mitigate the disadvantages of small agents, the inventor has created engineered agents to contain i) a therapeutic domain; ii) a cleavable domain; and iii) a stabilisation domain. This makes use of the advantages of larger agents (slow elimination and long half-lives) until the agent comes into contact with tumour-specific proteases in the vicinity of solid tumours, where cleavage of the agent causes reduction in the size of the agent. In the TME, the smaller parts of the agent (stabilisation domain and therapeutic domain) benefit from the advantageous pharmacokinetic properties discussed above.

The method according to the aspect above, wherein the improved efficacy of the agent is manifest by achieving the desired clinical result of the agent (such as reducing the proliferation of, destroying, reducing metastasis of cancerous cells or other diseased cells, shrinking the size of the tumour, decreasing symptoms resulting from the disease, increasing the quality of life of the subject, delaying the progression of the disease, and/or prolonging survival of the subject) using the same dosing regimen of the agent. Those skilled in the art would be capable of selecting an appropriate assay to measure the desired clinical result of the agent. For example, surgical removal of the solid tumour from the subject to measure the size of the tumour or fluorescent imaging of an in vivo tumour in a cancer animal model to visualise cell proliferation.

In another aspect, the invention provides a method of improving the specificity of an agent, the method comprising engineering the agent to comprise: i) a therapeutic domain; ii) a cleavable domain; and iii) a stabilisation domain wherein the cleavable domain is positioned between the therapeutic domain and the stabilising domain.

In the method according to the aspect above the improved specificity of the agent may be manifest by a higher titre of the therapeutic domain in diseased tissue compared to the titre of the therapeutic domain in non-diseased tissue. Those skilled in the art would be capable of selecting an appropriate assay to measure titre of the therapeutic domain in tissue. For example, biopsies from diseased tissue and non-diseased tissue could be taken. The titre of the therapeutic domain in each sample could then be assessed by immunostaining using antibodies (such as anti-CH1 antibodies, or anti-CL1 antibodies) or by ELISA.

The method may further comprise: i. applying the agent of the invention; ii. the cleavable domain being cleaved at the tumour; and iii. the therapeutic domain separating from the stabilisation domain. The terms “at the tumour” and “in the vicinity of the tumour” are used interchangeably.

In yet another aspect, the invention provides use of an agent comprising: i. a therapeutic domain; ii. a cleavable domain; and iii. a stabilisation domain to improve the penetrability of the therapeutic domain into a tumour.

In another aspect, the invention provides use of an agent comprising: i. a therapeutic domain; ii. a cleavable domain; and iii. a stabilisation domain to reduce the size of the agent at a tumour site. It will be appreciated that the size of the agent is reduced by specific cleavage of the cleavable domain. Cleavage of the agent at the tumour causes the formation of multiple smaller portions of the agent, which may comprise the stabilisation domain and therapeutic domain.

In yet another aspect, the invention provides use of an agent comprising: i. a therapeutic domain; ii. a cleavable domain; and iii. a stabilisation domain to improve the efficacy of the agent wherein the cleavable domain is positioned between the therapeutic domain and the stabilising domain.

In another aspect, the invention provides use of an agent comprising: i. a therapeutic domain; ii. a cleavable domain; and iii. a stabilisation domain to improve the specificity of the agent wherein the cleavable domain is positioned between the therapeutic domain and the stabilising domain.

In yet another aspect, the invention provides use of a cleavable domain to improve the penetrability of a therapeutic domain into a tumour, the use comprising engineering an agent to comprise: i. a therapeutic domain; ii. a cleavable domain; and iii. a stabilisation domain wherein the cleavable domain is positioned between the therapeutic domain and the stabilising domain.

In another aspect, the invention provides use of a cleavable domain to reduce the size of the agent at a tumour site, the use comprising engineering an agent to comprise: i. a therapeutic domain; ii. a cleavable domain; and iii. a stabilisation domain wherein the cleavable domain is positioned between the therapeutic domain and the stabilising domain.

In yet another aspect, the invention provides use of a cleavable domain to improve the efficacy of an agent, the use comprising engineering the agent to comprise: i. a therapeutic domain; ii. a cleavable domain; and iii. a stabilisation domain wherein the cleavable domain is positioned between the therapeutic domain and the stabilising domain.

In another aspect, the invention provides use of a cleavable domain to improve the specificity of an agent, the use comprising engineering the agent to comprise: i. a therapeutic domain; ii. a cleavable domain; and iii. a stabilisation domain wherein the cleavable domain is positioned between the therapeutic domain and the stabilising domain.

In an embodiment, the improved specificity of the agent is manifest by a higher titre of the therapeutic domain in diseased tissue compared to the titre of the therapeutic domain in non-diseased tissue. Preferably, the agent is the agent according to the first aspect of the invention wherein the cleavable domain is positioned between the therapeutic domain and the stabilising domain.

In yet another aspect, the invention provides the agent according to the first aspect of the invention for use in diagnosing a disease or disorder. By “diagnosis” we include the detection of cancer cells, either in vivo (i.e. within the body of a patient) or ex vivo (i.e. within a tissue or cell sample removed from the body of a patient). Those skilled in the art would be capable of selecting an appropriate assay to detect the cancer cells. As will be appreciated and as discussed herein, the agent, the therapeutic domain, the stabilisation domain may be detected by labelling (such as labelling the Fab region of an antibody with a fluorophore) and subsequent detection of the label (such as detection of the fluorophore using an antibody), by immunostaining using antibodies (such as anti-CH1 antibodies, anti-lambda antibodies, or anti-kappa antibodies) or by ELISA.

In another aspect of the invention the use comprises: i. administering the agent to a subject; ii. the cleavable domain being cleaved at the tumour; and iii. the therapeutic domain separating from the stabilisation domain; iv. increased accessibility to the cells for binding of the therapeutic domain compared to if cleavage and separation according to steps (ii) and (iii) had not occurred. By “accessibility to the cells for binding”, we include the meaning that some cells that previously had a limited ability to be accessed and be bound by parts of the agent (such as therapeutic domain or stabilisation domain) would be more accessible and more able to be bound by the parts of the agent. For example, prior to step (ii), the parts of the agent would bind to a limited extent to the interior parts of a tumour. After step (iii), the parts of the agent would bind to more of the interior parts of a tumour. Those skilled in the art would be capable of selecting an appropriate assay to assess accessibility to the cells for binding of the therapeutic domain. For example, the same methods used for measuring the ability of the agent to penetrate a solid tumour discussed above may be used.

Particularly preferably, selective cleavage of the cleavable domain enables the release of the therapeutic domain at or near to the cell surface of the unwanted cells. The terms “near to” and “in the vicinity of the tumour” are used interchangeably.

The use of the word “a” or “an” when used in conjunction with the term “comprising” in the claims and/or the specification may mean “one,” but it is also consistent with the meaning of “one or more,” “at least one,” and “one or more than one.”

Herein, when “about” is applied to a value or parameter, it includes (and describes) embodiments involving the value or parameter itself. For example, a description referring to “about X” includes a description of “X”. A numerical range includes the numbers defining the range. Although the numerical ranges and parameters setting forth the broad scope of the present disclosure are approximations, the numerical values set forth in the specific examples are reported as precisely as possible. However, any numerical values inherently contain certain errors necessarily resulting from the standard deviation found in their respective tests or measurements. Moreover, all ranges disclosed herein should be understood to encompass any and all subranges subsumed therein.

These, and other, embodiments of the invention will be better appreciated and understood when considered in conjunction with the above description and the accompanying drawings. It should be understood, however, that the above description, while indicating various embodiments of the invention and numerous specific details thereof, is given by way of illustration and not of limitation. Many substitutions, modifications, additions and/or rearrangements may be made within the scope of the invention without departing from the spirit thereof, and the invention includes all such substitutions, modifications, additions and/or rearrangements.

The listing or discussion in this specification of an apparently prior-published document should not necessarily be taken as an acknowledgement that the document is part of the state of the art or is common general knowledge.

### EXAMPLES

### Example 1: Antibody Designs

A cancer specific protease cleavage site (LSGRSDNH (SEQ ID NO: 77), emboldened below) for uPA was engineered in different regions of an exemplary antibody, trastuzumab, to enable physical separation of the therapeutic domain and the stabilisation domain at the tumour (FIG. 7). Seven different designs that included additions or substitutions of different regions of the antibody to the protease cleavage site are as follows:

In the engineered trastuzumab antibody design 1, the cleavage site LSGRSDNH (SEQ ID NO: 77) was added after the cysteine residue that ends the CH1 domain and prior to the DKTHT (SEQ ID NO: 78) hinge region (italicised). GS was added as an additional 2 amino acid linker to allow for greater accessibility.

Engineered trastuzumab antibody design 1
(SEQ ID NO: 1)
EVQLVESGGGLVQPGGSLRLSCAASGFNIKDTYIHWVRQAPGKGLEWVARIYPTNGYTRYADSVKGRFTI
SADTSKNTAYLQMNSLRAEDTAVYYCSRWGGDGFYAMDYWGQGTLVTVSSASTKGPSVFPLAPSSKSTS
GGTAALGCLVKDYFPEPVTVSWNSGALTSGVHTFPAVLQSSGLYSLSSVVTVPSSSLGTQTYICNVNHKP
SNTKVDKKVEPKSCGSLSGRSDNHDKTHTCPPCPAPELLGGPSVFLFPPKPKDTLMISRTPEVTCVVV
DVSHEDPEVKFNWYVDGVEVHNAKTKPREEQYNSTYRVVSVLTVLHQDWLNGKEYKCKVSNKALPAPIE
KTISKAKGQPREPQVYTLPPSREEMTKNQVSLTCLVKGFYPSDIAVEWESNGQPENNYKTTPPVLDSDGSF
FLYSKLTVDKSRWQQGNVFSCSVMHEALHNHYTQKSLSLSPGK

In the engineered trastuzumab antibody design 2, the cleavage site LSGRSDNH (SEQ ID NO: 77) was added after the cysteine residue that ends the CH1 domain and prior to the DKTHT (SEQ ID NO: 78) hinge region (italicised). No linker amino acids were added.

Engineered trastuzumab antibody design 2
(SEQ ID NO: 2)
EVQLVESGGGLVQPGGSLRLSCAASGFNIKDTYIHWVRQAPGKGLEWVARIYPTNGYTRYADSVKGRFTI
SADTSKNTAYLQMNSLRAEDTAVYYCSRWGGDGFYAMDYWGQGTLVTVSSASTKGPSVFPLAPSSKSTS
GGTAALGCLVKDYFPEPVTVSWNSGALTSGVHTFPAVLQSSGLYSLSSVVTVPSSSLGTQTYICNVNHKP
SNTKVDKKVEPKSCLSGRSDNHDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGKEYKCKVSNKALPAPIEKTIS
KAKGQPREPQVYTLPPSREEMTKNQVSLTCLVKGFYPSDIAVEWESNGQPENNYKTTPPVLDSDGSFFLYS
KLTVDKSRWQQGNVFSCSVMHEALHNHYTQKSLSLSPG

In the engineered trastuzumab antibody design 3, the cleavage site LSGRSDNH (SEQ ID NO: 77) was added after the cysteine residue that ends the CH1 domain and prior to the DKTHT (SEQ ID NO: 78) hinge region (italicised). GS was added as an additional 2 amino acid linker C and N terminal to the protease cleavage site for greater accessibility.

Engineered trastuzumab antibody design 3
(SEQ ID NO: 3)
EVQLVESGGGLVQPGGSLRLSCAASGFNIKDTYIHWVRQAPGKGLEWVAR
IYPTNGYTRYADSVKGRFTISADTSKNTAYLQMNSLRAEDTAVYYCSRWG
GDGFYAMDYWGQGTLVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKKVEPKSCGSLSGRSDNHGSDKTHTCPPCPAPELL
GGPSVFLFPPKPKDTLMISRTPEVTCVVVDVSHEDPEVKFNWYVDGVEVH
NAKTKPREEQYNSTYRVVSVLTVLHQDWLNGKEYKCKVSNKALPAPIEKT
ISKAKGQPREPQVYTLPPSREEMTKNQVSLTCLVKGFYPSDIAVEWESNG
QPENNYKTTPPVLDSDGSFFLYSKLTVDKSRWQQGNVFSCSVMHEALHNH
YTQKSLSLSPGK

In the engineered trastuzumab antibody design 4, the DKTHT (SEQ ID NO: 78) hinge region was substituted with LSGRSDNH (SEQ ID NO: 77) after the cysteine residue that ends the CH1 domain and prior to the hinge disulphide bond region (CPPC (SEQ ID NO: 79)). This is the minimal sequence modification needed to insert a protease cleavage site.

Engineered trastuzumab antibody design 4
(SEQ ID NO: 4)
EVQLVESGGGLVQPGGSLRLSCAASGFNIKDTYIHWVRQAPGKGLEWVAR
IYPTNGYTRYADSVKGRFTISADTSKNTAYLQMNSLRAEDTAVYYCSRWG
GDGFYAMDYWGQGTLVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKKVEPKSCLSGRSDNHCPPCPAPELLGGPSVFLFP
PKPKDTLMISRTPEVTCVVVDVSHEDPEVKFNWYVDGVEVHNAKTKPREE
QYNSTYRVVSVLTVLHQDWLNGKEYKCKVSNKALPAPIEKTISKAKGQPR
EPQVYTLPPSREEMTKNQVSLTCLVKGFYPSDIAVEWESNGQPENNYKTT
PPVLDSDGSFFLYSKLTVDKSRWQQGNVFSCSVMHEALHNHYTQKSLSLS
PGK

In the engineered trastuzumab antibody design 5, the DKTHT (SEQ ID NO: 78) hinge region was substituted with LSGRSDNHT (SEQ ID NO: 80) after the cysteine residue that ends the CH1 domain and prior to the hinge disulphide bond region (CPPC (SEQ ID NO: 79)). The additional threonine was thought to provide a similar neighbouring environment to the cysteine of the hinge region to allow that region to fold correctly.

Engineered trastuzumab antibody design 5
(SEQ ID NO: 5)
EVQLVESGGGLVQPGGSLRLSCAASGFNIKDTYIHWVRQAPGKGLEWVAR
IYPTNGYTRYADSVKGRFTISADTSKNTAYLQMNSLRAEDTAVYYCSRWG
GDGFYAMDYWGQGTLVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKKVEPKSCLSGRSDNHTCPPCPAPELLGGPSVFLF
PPKPKDTLMISRTPEVTCVVVDVSHEDPEVKFNWYVDGVEVHNAKTKPRE
EQYNSTYRVVSVLTVLHQDWLNGKEYKCKVSNKALPAPIEKTISKAKGQP
REPQVYTLPPSREEMTKNQVSLTCLVKGFYPSDIAVEWESNGQPENNYKT
TPPVLDSDGSFFLYSKLTVDKSRWQQGNVFSCSVMHEALHNHYTQKSLSL
SPGK

In the engineered trastuzumab antibody design 6, LSGRSDNH (SEQ ID NO: 77) was added to the CH2 domain three amino acids (PAP) after the hinge disulphide bond region (CPPC (SEQ ID NO: 79)) (to give a F(ab′)2 fragment) to allow accessibility of the protease.

Engineered trastuzumab antibody design 6
(SEQ ID NO: 6)
EVQLVESGGGLVQPGGSLRLSCAASGFNIKDTYIHWVRQAPGKGLEWVAR
IYPTNGYTRYADSVKGRFTISADTSKNTAYLQMNSLRAEDTAVYYCSRWG
GDGFYAMDYWGQGTLVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKKVEPKSCDKTHTCPPCPAPLSGRSDNHELLGGPS
VFLFPPKPKDTLMISRTPEVTCVVVDVSHEDPEVKFNWYVDGVEVHNAKT
KPREEQYNSTYRVVSVLTVLHQDWLNGKEYKCKVSNKALPAPIEKTISKA
KGQPREPQVYTLPPSREEMTKNQVSLTCLVKGFYPSDIAVEWESNGQPEN
NYKTTPPVLDSDGSFFLYSKLTVDKSRWQQGNVFSCSVMHEALHNHYTQK
SLSLSPGK

In the engineered trastuzumab antibody design 7, ELLGGPSV (SEQ ID NO: 81) was substituted with LSGRSDNH (SEQ ID NO: 77) three amino acids (PAP) after the hinge disulphide bond region (CPPC (SEQ ID NO: 79)). This is the minimal mutation that could be made to give a F(ab′)2 fragment.

Engineered trastuzumab antibody design 7
(SEQ ID NO: 7)
EVQLVESGGGLVQPGGSLRLSCAASGFNIKDTYIHWVRQAPGKGLEWVAR
IYPTNGYTRYADSVKGRFTISADTSKNTAYLQMNSLRAEDTAVYYCSRWG
GDGFYAMDYWGQGTLVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKKVEPKSCDKTHTCPPCPAPLSGRSDNHFLFPPKP
KDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPV
LDSDGSFFLYSKLTVDKSRWQQGNVFSCSVMHEALHNHYTQKSLSLSPGK

### Example 2: Molecular Biology

### Engineered Trastuzumab Geneblock Designs 1-3

Geneblocks of trastuzumab were synthesised (Integrated DNA Technologies) with Engineered trastuzumab geneblock designs 1 to 3, as given below:

Engineered trastuzumab geneblock design 1
(SEQ ID NO: 82)
attaagctcttccctggccGAGGTTCAACTGGTTGAGAGTGGAGGGGGAT
TGGTGCAGCCAGGCGGCAGTCTGCGGTTGAGCTGCGCAGCCTCTGGATTC
AATATCAAAGATACTTACATACACTGGGTCCGACAGGCACCTGGCAAGGG
GCTTGAGTGGGTTGCCCGTATTTATCCCACTAACGGTTATACACGGTACG
CCGACTCTGTTAAAGGTCGATTCACAATAAGTGCAGATACCTCCAAAAAC
ACAGCTTATCTGCAAATGAACAGCCTTCGCGCAGAAGATACTGCAGTTTA
TTATTGCTCACGCTGGGGTGGGGATGGCTTCTACGCTATGGACTATTGGG
GGCAGGGCACCCTGGTCACTGTATCCTCTGCCTCTACAAAAGGTCCATCC
GTATTCCCATTGGCTCCAAGCTCTAAGAGTACTTCTGGTGGTACAGCCGC
CCTTGGATGTCTCGTAAAAGATTACTTCCCTGAACCTGTGACCGTCTCCT
GGAATTCAGGGGCACTGACAAGCGGCGTTCATACTITTCCTGCCGTTCTT
CAGTCAAGTGGCCTTTACAGTCTGAGTTCCGTCGTAACAGTCCCTAGCTC
AAGTCTTGGGACACAGACTTATATCTGCAACGTAAATCACAAACCCTCTA
ATACTAAGGTAGACAAGAAAGTGGAGCCCAAATCCTGTGGGTCTCTCTCC
GGTCGATCCGACAACCACgctggaagagcgg
Engineered trastuzumab geneblock design 2
(SEQ ID NO: 83)
attaagctcttccctggccGAGGTTCAACTGGTTGAGAGTGGAGGGGGAT
TGGTGCAGCCAGGCGGCAGTCTGCGGTTGAGCTGCGCAGCCTCTGGATTC
AATATCAAAGATACTTACATACACTGGGTCCGACAGGCACCTGGCAAGGG
GCTTGAGTGGGTTGCCCGTATTTATCCCACTAACGGTTATACACGGTACG
CCGACTCTGTTAAAGGTCGATTCACAATAAGTGCAGATACCTCCAAAAAC
ACAGCTTATCTGCAAATGAACAGCCTTCGCGCAGAAGATACTGCAGTTTA
TTATTGCTCACGCTGGGGTGGGGATGGCTTCTACGCTATGGACTATTGGG
GGCAGGGCACCCTGGTCACTGTATCCTCTGCCTCTACAAAAGGTCCATCC
GTATTCCCATTGGCTCCAAGCTCTAAGAGTACTTCTGGTGGTACAGCCGC
CCTTGGATGTCTCGTAAAAGATTACTTCCCTGAACCTGTGACCGTCTCCT
GGAATTCAGGGGCACTGACAAGCGGCGTTCATACTTTTCCTGCCGTTCTT
CAGTCAAGTGGCCTTTACAGTCTGAGTTCCGTCGTAACAGTCCCTAGCTC
AAGTCTTGGGACACAGACTTATATCTGCAACGTAAATCACAAACCCTCTA
ATACTAAGGTAGACAAGAAAGTGGAGCCCAAATCCTGTCTCTCCGGTCGA
TCCGACAACCACgctggaagagcgg
Engineered trastuzumab geneblock design 3
(SEQ ID NO: 84)
attaagctcttccctggccGAGGTTCAACTGGTTGAGAGTGGAGGGGGAT
TGGTGCAGCCAGGCGGCAGTCTGCGGTTGAGCTGCGCAGCCTCTGGATTC
AATATCAAAGATACTTACATACACTGGGTCCGACAGGCACCTGGCAAGGG
GCTTGAGTGGGTTGCCCGTATTTATCCCACTAACGGTTATACACGGTACG
CCGACTCTGTTAAAGGTCGATTCACAATAAGTGCAGATACCTCCAAAAAC
ACAGCTTATCTGCAAATGAACAGCCTTCGCGCAGAAGATACTGCAGTTTA
TTATTGCTCACGCTGGGGTGGGGATGGCTTCTACGCTATGGACTATTGGG
GGCAGGGCACCCTGGTCACTGTATCCTCTGCCTCTACAAAAGGTCCATCC
GTATTCCCATTGGCTCCAAGCTCTAAGAGTACTTCTGGTGGTACAGCCGC
CCTTGGATGTCTCGTAAAAGATTACTTCCCTGAACCTGTGACCGTCTCCT
GGAATTCAGGGGCACTGACAAGCGGCGTTCATACTTTTCCTGCCGTTCTT
CAGTCAAGTGGCCTTTACAGTCTGAGTTCCGTCGTAACAGTCCCTAGCTC
AAGTCTTGGGACACAGACTTATATCTGCAACGTAAATCACAAACCCTCTA
ATACTAAGGTAGACAAGAAAGTGGAGCCCAAATCCTGTGGGTCTCTCTCC
GGTCGATCCGACAACCACGGGTCTgctggaagagcgg

All inserts contained the type II restriction enzyme site SapI to enable high throughput cloning.

Destination vectors were modified with SapI restriction sites to enable VH+/CH1 domains to be cloned in via a high throughput method. The destination vectors are for mammalian expression of antibodies and include a hinge and an Fc region.

100 ng of each geneblock was mixed with 100 ng of pCREA high throughput mammalian Fc vectors along with 5 units of SapI restriction enzyme (New England Biolabs) and 200 units of T4 DNA ligase (New England Biolabs). The samples were left to incubate for 60 minutes at 37° C., then deactivation of the enzyme at 65° C. for 20 minutes. The DNA was transformed into NEB 5 alpha cells (New England Biolabs) and plated on LB agar plates containing 100 μg/ml of carbenicillin. Plates were incubated at 37° C. overnight. 5 colonies were picked the following day and the correct inserts were checked by mini-prep followed by Sanger sequencing. For large scale preparation of DNA for transfection, the correct constructs were prepped using the endotoxin free maxi prep kit (Qiagen).

### Engineered Trastuzumab Geneblock Designs 4-7

As designs 4-7 were of closer proximity to the hinge, these constructs were prepared by making geneblocks (Integrated DNA Technologies) that spanned over CH1 and CH2 and are as follows.

Engineered trastuzumab geneblock
(CH1 CH2) design 4
(SEQ ID NO: 85)
atgccattgtaACCGGTGACGGTGTCGTGGAACTCAGGCGCCCTGACCAG
CGGCGTGCACACCTTCCCGGCTGTCCTACAGTCCTCAGGACTCTACTCCC
TCAGCAGCGTGGTGACCGTGCCCTCCAGCAGCTTGGGCACCCAGACCTAC
ATCTGCAACGTGAATCACAAGCCCAGCAACACCAAGGTGGACAAGAGAGT
TGAGCCCAAATCTTGTCTCTCCGGTCGATCCGACAACCACTGCCCACCGT
GCCCAGCACCTGAACTCCTGGGGGGACCGTCAGTCTTCCTCTTCCCCCCA
AAACCCAAGGACACCCTCATGATCTCCCGGACCCCTGAGGTCACATGCGT
GGTGGTGGACGTGAGCCACGAAGACCCTGAGGTCAAGTTCAACTGGTACG
TGGACGGCGTGGAGGTGCATAATGCCAAGACAAAGCCGCGGGAGGAGCAG
TACAACAGCACGTACCGTGTGGTCAGCGTCCTCACCGTCCTGCACCAGGA
CTGGCTGAATGGCAAGGAGTACAAGTGCAAGGTCTCCAACAAAGCCCTCC
CAGCCCCCATCGAGAAAACCATCTCCAAAGCCAAAGGGCAGCCCCGAGAA
CCACAGGTGTACACCCTGCCCCCATCCCGGGAGGAGATGACCAAGAACCA
GGTattcgcaatc
Engineered trastuzumab geneblock
(CH1 CH2) design 5
(SEQ ID NO: 86)
atgccattgtaACCGGTGACGGTGTCGTGGAACTCAGGCGCCCTGACCAG
CGGCGTGCACACCTTCCCGGCTGTCCTACAGTCCTCAGGACTCTACTCCC
TCAGCAGCGTGGTGACCGTGCCCTCCAGCAGCTTGGGCACCCAGACCTAC
ATCTGCAACGTGAATCACAAGCCCAGCAACACCAAGGTGGACAAGAGAGT
TGAGCCCAAATCTTGTCTCTCCGGTCGATCCGACAACCACacaTGCCCAC
CGTGCCCAGCACCTGAACTCCTGGGGGGACCGTCAGTCTTCCTCTTCCCC
CCAAAACCCAAGGACACCCTCATGATCTCCCGGACCCCTGAGGTCACATG
CGTGGTGGTGGACGTGAGCCACGAAGACCCTGAGGTCAAGTTCAACTGGT
ACGTGGACGGCGTGGAGGTGCATAATGCCAAGACAAAGCCGCGGGAGGAG
CAGTACAACAGCACGTACCGTGTGGTCAGCGTCCTCACCGTCCTGCACCA
GGACTGGCTGAATGGCAAGGAGTACAAGTGCAAGGTCTCCAACAAAGCCC
TCCCAGCCCCCATCGAGAAAACCATCTCCAAAGCCAAAGGGCAGCCCCGA
GAACCACAGGTGTACACCCTGCCCCCATCCCGGGAGGAGATGACCAAGAA
CCAGGTattcgcaatctc
Engineered trastuzumab geneblock
(CH1 CH2) design 6
(SEQ ID NO: 87)
atgccattgtaACCGGTGACGGTGTCGTGGAACTCAGGCGCCCTGACCAG
CGGCGTGCACACCTTCCCGGCTGTCCTACAGTCCTCAGGACTCTACTCCC
TCAGCAGCGTGGTGACCGTGCCCTCCAGCAGCTTGGGCACCCAGACCTAC
ATCTGCAACGTGAATCACAAGCCCAGCAACACCAAGGTGGACAAGAGAGT
TGAGCCCAAATCTTGTGACAAAACTCACACATGCCCACCGTGCCCAGCAC
TCTCCGGTCGATCCGACAACCACCCTGAACTCCTGGGGGGACCGTCAGTC
TTCCTCTTCCCCCCAAAACCCAAGGACACCCTCATGATCTCCCGGACCCC
TGAGGTCACATGCGTGGTGGTGGACGTGAGCCACGAAGACCCTGAGGTCA
AGTTCAACTGGTACGTGGACGGCGTGGAGGTGCATAATGCCAAGACAAAG
CCGCGGGAGGAGCAGTACAACAGCACGTACCGTGTGGTCAGCGTCCTCAC
CGTCCTGCACCAGGACTGGCTGAATGGCAAGGAGTACAAGTGCAAGGTCT
CCAACAAAGCCCTCCCAGCCCCCATCGAGAAAACCATCTCCAAAGCCAAA
GGGCAGCCCCGAGAACCACAGGTGTACACCCTGCCCCCATCCCGGGAGGA
GATGACCAAGAACCAGGTattcgcaatc
Engineered trastuzumab geneblock
(CH1 CH2) design 7
(SEQ ID NO: 88)
atgccattgtaACCGGTGACGGTGTCGTGGAACTCAGGCGCCCTGACCAG
CGGCGTGCACACCTTCCCGGCTGTCCTACAGTCCTCAGGACTCTACTCCC
TCAGCAGCGTGGTGACCGTGCCCTCCAGCAGCTTGGGCACCCAGACCTAC
ATCTGCAACGTGAATCACAAGCCCAGCAACACCAAGGTGGACAAGAGAGT
TGAGCCCAAATCTTGTGACAAAACTCACACATGCCCACCGTGCCCAGCAC
TCTCCGGTCGATCCGACAACCACTTCCTCTTCCCCCCAAAACCCAAGGAC
ACCCTCATGATCTCCCGGACCCCTGAGGTCACATGCGTGGTGGTGGACGT
GAGCCACGAAGACCCTGAGGTCAAGTTCAACTGGTACGTGGACGGCGTGG
AGGTGCATAATGCCAAGACAAAGCCGCGGGAGGAGCAGTACAACAGCACG
TACCGTGTGGTCAGCGTCCTCACCGTCCTGCACCAGGACTGGCTGAATGG
CAAGGAGTACAAGTGCAAGGTCTCCAACAAAGCCCTCCCAGCCCCCATCG
AGAAAACCATCTCCAAAGCCAAAGGGCAGCCCCGAGAACCACAGGTGTAC
ACCCTGCCCCCATCCCGGGAGGAGATGACCAAGAACCAGGTattcgcaat
c

The 5′ restriction enzyme as AgeI and 3′ was BsrGI. The destination vector was trastuzumab with Engineered trastuzumab antibody design 1 (SEQ ID NO: 1).

The geneblocks were cut with AgeI-HF (New England Biolabs) and BsrGI (New England Biolabs), as was the vector trastuzumab in the pCREA vector. The samples were left to digest overnight at 37° C. The geneblocks were purified by a PCR purification kit, but the vector was run on a 1% agarose gel in Tris-acetate-EDTA (TAE) buffer to separate the insert from the backbone. The larger backbone fragment was band extracted. Ligations were set up in a 3:1 molar ratio of insert to backbone of the cut geneblocks and cut backbone. Ligations were with 400 units of T4 ligase (New England Biolabs) and were left overnight at room temperature. Ligations were transformed into NEB 5 alpha cells (New England Biolabs) and spread onto LB agar plates with 100 μg/ml of ampicillin. 5 colonies were checked for the presence of the correct insert by mini prep followed by Sanger sequencing. For large scale preparation of DNA for transfection, the correct constructs were prepped using the endotoxin free maxi prep kit (Qiagen).

### Example 3: Antibody Expression and Purification

### CHO-K1 Cell Lines

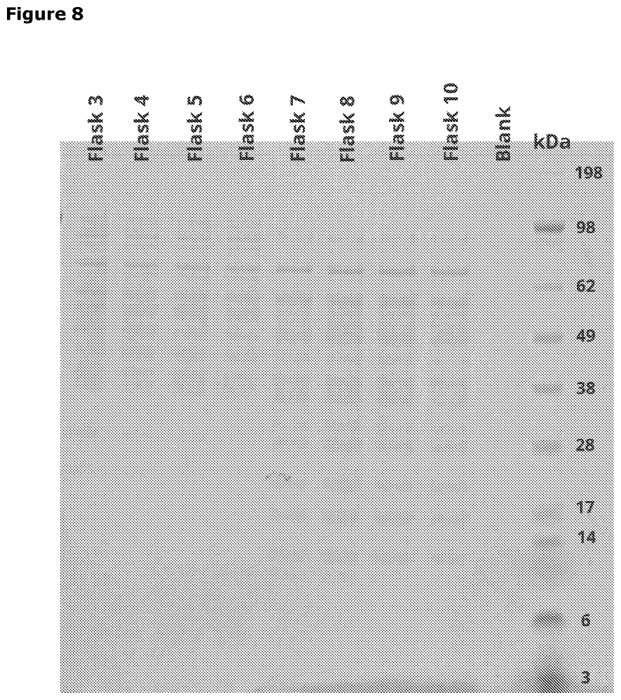

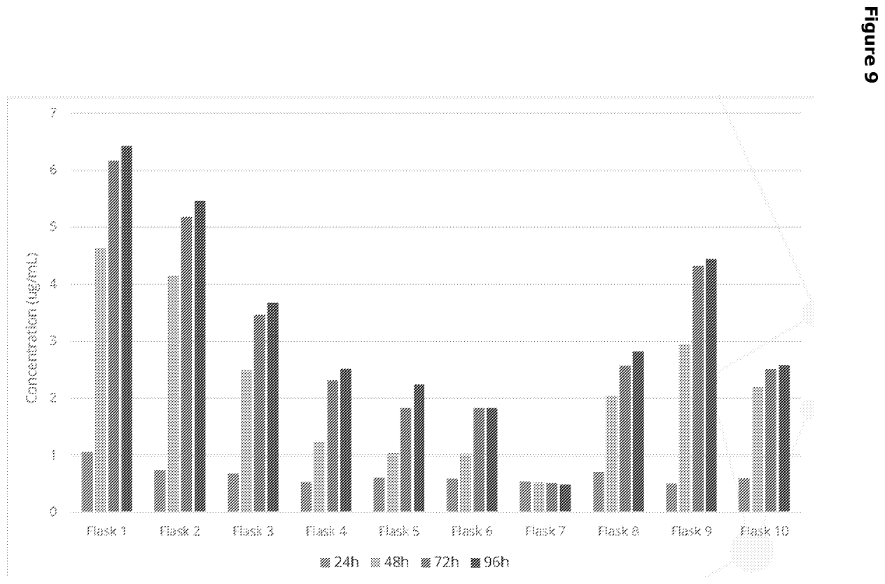

Trastuzumab in the pCREA antibody mammalian expression vector, alongside the engineered trastuzumab antibody design 1 in the pCREA antibody mammalian expression vector were transfected into CHO-K1 cells by either polyethylenimine (PEI) or expifectamine in a 3:1 ratio of DNA to PEI only in 30 ml shake flasks. Cells were grown for up to 7 days (96 hours post-transfection), with monitoring of cell health and titre from days 1-4. Comparison of cell health over the first 4 days shows that the engineered trastuzumab antibody design 1 (D1) is indistinguishable from trastuzumab monoclonal antibody. Assessment of antibody concentration by Protein A Octet revealed that there are no differences between the monoclonal antibody and design 1 equivalent for trastuzumab (as shown in Table 1 and FIG. 7). At 96 hours post-transfection, expression levels were between 0.891 and 3.02 mg/L. SDS-PAGE was used to look for overexpression, however as expression levels were overall low, this could not be detected (as shown in FIG. 8).

TABLE 1
Summary of antibody titre

| | Transfection | | 96h Octet |
|---|---|---|---|
| Flask no. | Method | Condition | Conc. (μg/mL) |
| Flask 3 | PEI | Control mAb | 3.42 |
| Flask 4 | PEI | Control mAb | 3.67 |
| Flask 5 | PEI | Crea-mAb | 3.02 |
| Flask 6 | PEI | Crea-mAb | 3.01 |
| Flask 7 | Expifectamine | Control mAb | 0.894 |
| Flask 8 | Expifectamine | Control mAb | 0.891 |
| Flask 9 | Expifectamine | Crea-mAb | 0.924 |
| Flask 10 | Expifectamine | Crea-mAb | 0.964 |

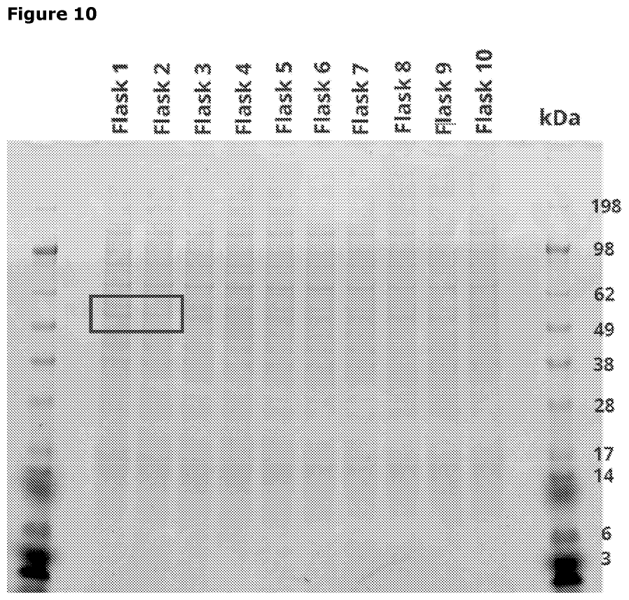

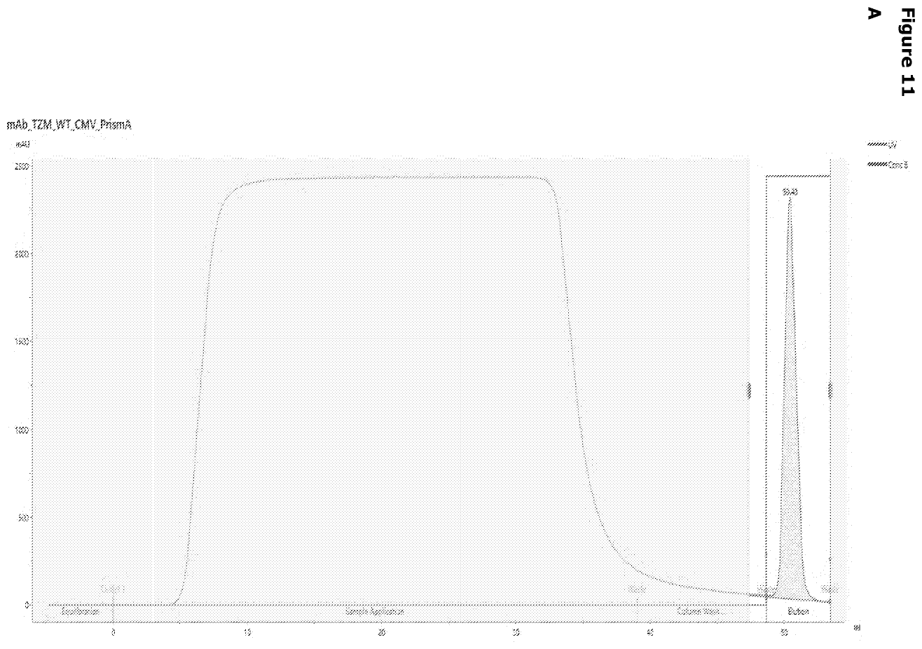

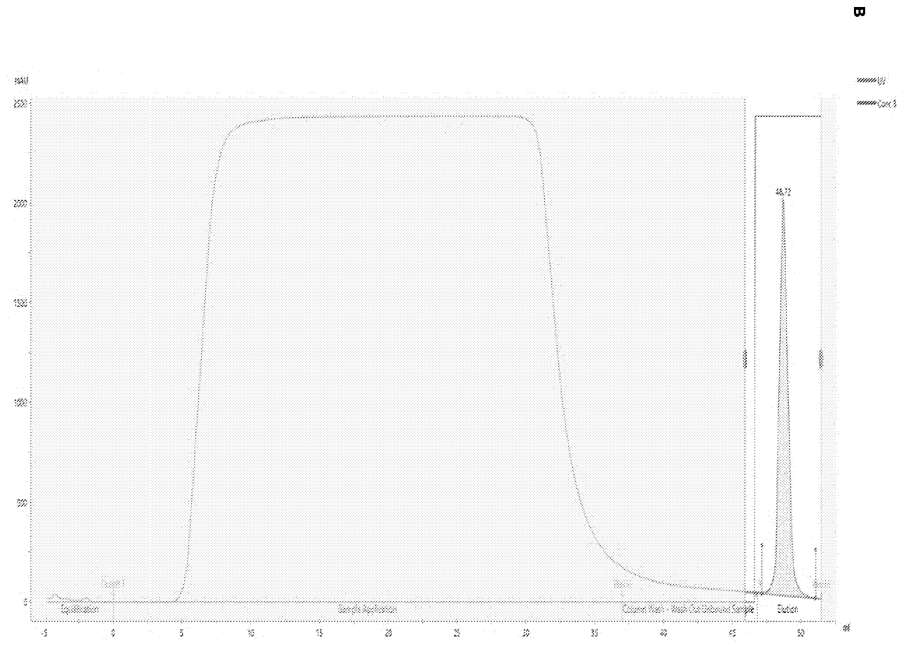

Transfection conditions were optimised, and cells were grown for up to 7 days, with monitoring of cell health and titre from days 1-4. Again, comparison of cell health over the first 4 days shows that the engineered trastuzumab antibody design 1 is indistinguishable from trastuzumab monoclonal antibody. Assessment of antibody concentration by Protein A Octet revealed that there are no differences between the monoclonal antibody and design 1 equivalent for trastuzumab (as shown in Table 2 and FIG. 9). At 96 hours post-transfection, expression levels were between 1.83 and 5.46 mg/L. SDS-PAGE was used to look for overexpression and overexpression of the antibody heavy chains could be detected (see box in FIG. 10).

TABLE 2
Summary of antibody titre in optimised experiment

| | | | | 48 h | 72 h | 96 h |
|---|---|---|---|---|---|---|
| | Trans- | | 24 h | Octet | Octet | Octet |
| | fection | | Conc. | Conc. | Conc. | Conc. |
| Flask no. | Method | Condition | (ug/mL) | (μg/mL) | (μg/mL) | (μg/mL) |
| Flask 1 | PEI | Control | 1.06 | 4.63 | 6.16 | 6.42 |
| | | mAb | | | | |
| Flask 2 | PEI | Crea-mAb | 0.74 | 4.15 | 5.18 | 5.46 |
| Flask 3 | PEI | Control | 0.68 | 2.49 | 3.46 | 3.67 |
| | | mAb | | | | |
| Flask 4 | PEI | Crea-mAb | 0.53 | 1.24 | 2.31 | 2.51 |
| Flask 5 | PEI | Control | 0.60 | 1.04 | 1.83 | 2.24 |
| | | mAb | | | | |
| Flask 6 | PEI | Crea-mAb | 0.59 | 1.02 | 1.83 | 1.83 |
| Flask 7 | PEI | Control | 0.54 | 0.53 | 0.51 | 0.49 |
| | | mAb | | | | |
| Flask 8 | PEI | Crea-mAb | 0.70 | 2.04 | 2.57 | 2.81 |
| Flask 9 | PEI | Control | 0.50 | 2.94 | 4.32 | 4.44 |
| | | mAb | | | | |
| Flask 10 | PEI | Crea-mAb | 0.59 | 2.19 | 2.51 | 2.58 |

### Expi-CHO Cell Lines

After cloning, the trastuzumab in the pCREA antibody mammalian expression vector, alongside engineered trastuzumab antibody designs 1 to 7 (D1 to D7) in the pCREA antibody mammalian expression vector, were diluted to a concentration of 1 μg/μl and then transfected into Expi-CHO cell lines (Invitrogen), by Optipro+ ExpiFectamine in shake flasks and incubated at 37° C. overnight. The cultures were fed and then the cells were grown for 9 days.

### Purification

On day 9 the cells were harvested by spinning them down at 3000×g for 30 minutes and filtered through a 0.2 μm filter to recover the overexpressed antibody from the cell supernatants. The supernatants were purified on MabSelect PrismA 1 ml prepacked columns using the AKTA Go Instrument (Cytiva). Elution was carried out at pH 2.7 in HiTrap® MabSelect Binding Buffer (NaPO₄) and HiTrap® MabSelect Elution Buffer (Glycine). The relevant fractions were pooled according to chromatograms and the buffer exchanged in 1×DPBS using Vivaspin20 concentrators at 3800×g for 20 minutes (as shown in FIG. 11 for D1). The absorbance was measured using a Nanodrop (ThermoScientific), and the yield and concentration was calculated.

Trastuzumab and engineered antibodies_D1-7 have similar expression levels and yields (as shown in Table 3).

TABLE 3
Summary of antibody expression level and yield

| | Conc. | Conc. | Volume | Yield | Expression |
|---|---|---|---|---|---|
| Sample | (μM) | (mg/ml) | (ml) | (mg) | Level (mg/L) |
| mAb | 9.47 | 1.38 | 4.8 | 6.64 | 265.6 |
| D1 | 5.53 | 0.81 | 5.0 | 4.04 | 161.6 |
| D2 | 14.90 | 2.18 | 4.9 | 10.67 | 426.9 |
| D3 | 13.68 | 2.00 | 5.0 | 10.00 | 400.0 |
| D4 | 15.75 | 2.30 | 5.4 | 12.43 | 497.1 |
| D5 | 12.84 | 1.88 | 5.4 | 10.13 | 405.4 |
| D6 | 16.45 | 2.40 | 4.2 | 10.10 | 403.9 |
| D7 | 6.09 | 0.89 | 6.0 | 5.34 | 213.7 |

### SDS-Page Analysis of Purified Control and D1-D7

The purified antibodies were heated at 85° C. for 5 minutes, then 10 μl of which were loaded onto a NuPAGE 4-12% Bis-Tris Gel inserted in a mini Gel Tank filled with 1×MES SDS running buffer, with a protein marker loaded as molecular weight standard. The gel was run for 25 minutes at 200 V, then stained with InstantBlue® protein stain and subsequently destained with H₂O.

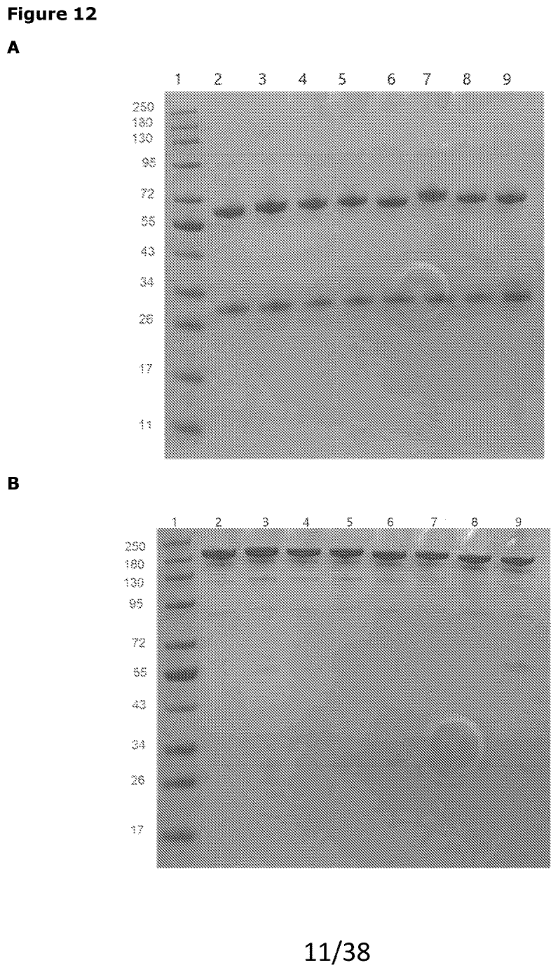

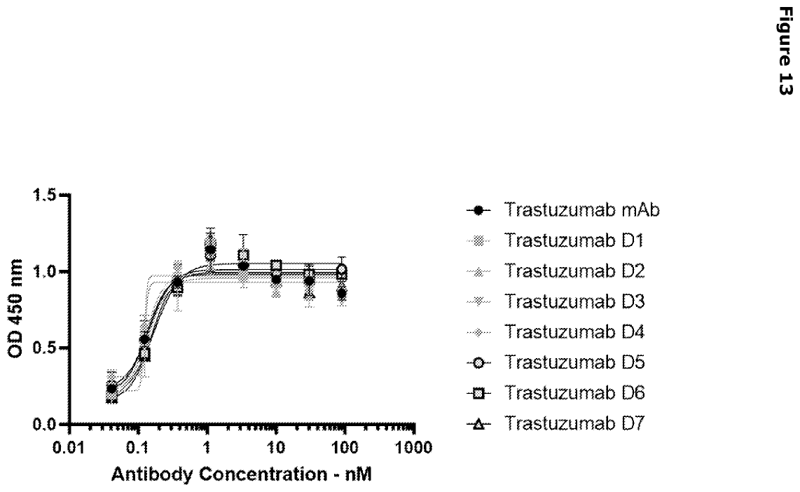

Both the reduced and non-reduced SDS-PAGE gels showed mAb-like antibody profiles (as shown in FIG. 12). All molecules appeared as expected, with D5 being the biggest variant as it has a higher molecular weight due to the engineered glycosylation site. These results are consistent with the results observed in the size exclusion (SEC) high-performance liquid chromatography (HPLC) assays and Tₘ and T_{agg} determination assays analysis below.

### SEC-HPLC Assay of Purified Engineered Antibodies

10 μg of the purified antibody samples were injected into a MAbPac SEC-1 (4×300 mm) column with precolumn MabPac SEC-1 (4×50 mm) and a SECHPLC assay was performed to determine the percentage monomer to higher order oligomers present.

The SEC-HPLC assay demonstrated good solution state profiles of D1-7 (as shown in Table 4) showing that they all had acceptable levels of purity comparative to the control, trastuzumab.

TABLE 4
Thermal Stability Analysis of mAb Control and D1-7

| | | Monomer | DSF | | SLS |
|---|---|---|---|---|---|
| Sample | | relative area | T_{M1} | T_{M2} | T _{AGG} |
| ID | Buffer | (%) | (° C.) | (° C.) | (° C.) |
| mAb | DPBS | 64.1 | 69.8 | — | 67.1 |
| control | | | | | |
| D1 | | 39.8 | 69.3 | — | 70.5 |
| D2 | | 69.7 | 70.1 | — | 71.1 |
| D3 | | 96.9 | 70.7 | — | 70.7 |
| D4 | | 96.4 | 70.8 | — | 67.8 |
| D5 | | 89.6 | 69.9 | — | 74.5 |
| D6 | | 99.3 | 68.4 | — | 67.8 |
| D7 | | 52.0 | 47.4 | 76.1 | 68.3 |

### Thermal Stability of Purified Engineered Antibodies

The samples were diluted to 1 mg/mL in DPBS (Dulbecco's Phosphate Buffered Saline), then 9 μL of each sample was loaded in triplicate into the UNCLE instrument (Unchained Labs) and the melting temperature (Tₘ) and aggregation temperature (T_{agg}) were determined.

All antibodies showed high T_{M} and T_{agg} values (Table 4). All antibodies presented T_{M} values above 65.0° C., therefore meeting the usual requirement for CMC development, except D7 which showed the lowest TM of 47.4° C. (as shown in Table 4). D7 was also the only sample to exhibit a second T_{M} (76.1° C.). D4 presented the highest T_{M1} value (70.8° C.), while D7 showed the lowest (47.4° C.). The highest T_{agg} was observed for D5 (74.5° C.), and the lowest for the trastuzumab control (67.1° C.).

### Conclusion

Considering the analysed biophysical parameters, engineered D1-D7 have been shown to maintain the biophysical and biochemical properties required of trastuzumab for functioning as an antibody and such properties have not been negatively impacted by the engineering of adding the cleavable site.

### Example 4: Antibody Characterisation

Binding of purified engineered antibodies to HER2 (assessed by ELISA) Maxisorp™ ELISA plates were prepared by adding 50 μl of either phosphate buffered saline (PBS), bovine serum albumin (BSA—1 ug/ml) or hHER2 (1 ug/ml) to each well and placed in the fridge overnight. The plates were washed three times with PBS+0.1% Tween20 and three times in PBS and left to block with 200 μl of PBS+6% dried milk for one hour at room temperature before being washed again.

The purified trastuzumab mAb and _D1-7 antibodies were diluted to 50 μl per well in PBS+3% dried milk, then added to the plates and incubated for 1 hour at room temperature, after which the plates were washed again. Antibodies were added at a top concentration of 150-50 nM and diluted 1 in 2, or 1 in 3 over an 8 or 12 point dilution series. 50 μl of a 1 in 5000 dilution in PBS+3% dried milk of the anti Vk antibody-HRP conjugated was added to each well, then left to bind to the samples at room temperature for 1 hour. The plates were washed again, and 50 μl of 3,3′,5,5′-Tetramethylbenzidine (TMB) peroxidase substrate was added to develop the colour. The signal was quenched with 50 μl of 1M HCl and the plates were read for absorbance at 450 nm.

D1-D7 show identical binding to hHER2 by ELISA (as shown in Table 5 and FIG. 13).

TABLE 5
IC₅₀ values of trastuzumab control and D1-D7

| Antibody | IC₅₀ |
|---|---|
| Trastuzumab mAb | 0.1325 |
| Trastuzumab D1 | 0.1282 |
| Trastuzumab D2 | 0.1239 |
| Trastuzumab D3 | 0.1395 |
| Trastuzumab D4 | 0.1235 |
| Trastuzumab D5 | 0.1423 |
| Trastuzumab D6 | 0.1679 |
| Trastuzumab D7 | 0.1548 |

### Binding of Purified Engineered Antibodies to HER2 (Assessed by BIAcore)

A CM5 chip was coated with anti-human IgG with 12,500 RU units, using conditions of Cytiva® HBS-EP (0.01 M HEPES pH 7.4, 0.15 M NaCl, 3 mM EDTA, 0.005% v/v Surfactant P20)+running buffer and regeneration with 0.1 M glycine pH 2.0. The hHER2 protein did not show binding to the blank CM5 chip.

Anti-Human IgG Fc antibody was then immobilised to 10,215.8 RU units on flow cell (Fc) 2 of a CM5 chip using amine coupling chemistry, using HBS-EP+ running buffer. HER2 protein was then shown not to bind to the immobilised anti-human IgG Fc antibody.

Capture optimisation of trastuzumab (at 0.1, 0.5 and 1 μg/ml) using hHER2 (at 1, 10 and 100 nM) was performed. Trastuzumab showed stable capture levels and capture concentrations, and its capture baseline did not vary much between cycles which implied efficient regeneration using 0.1M glycine HCl pH 2.0.

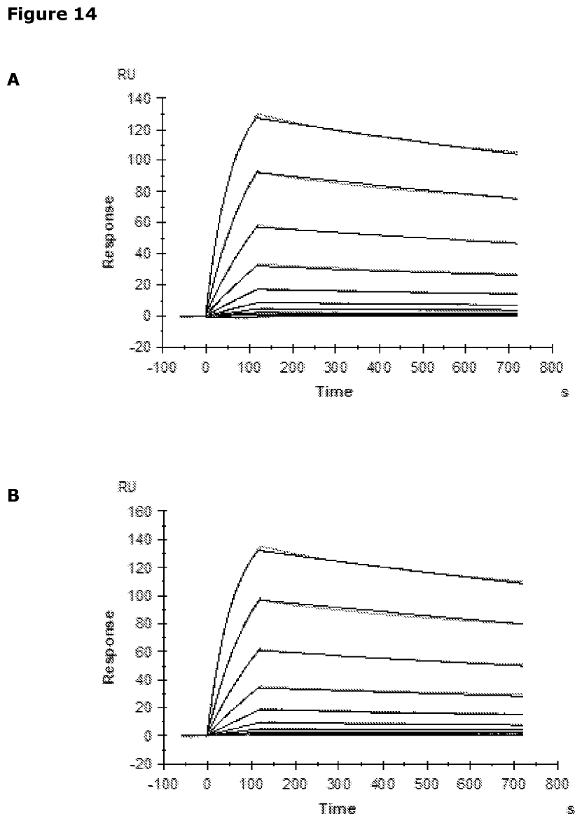

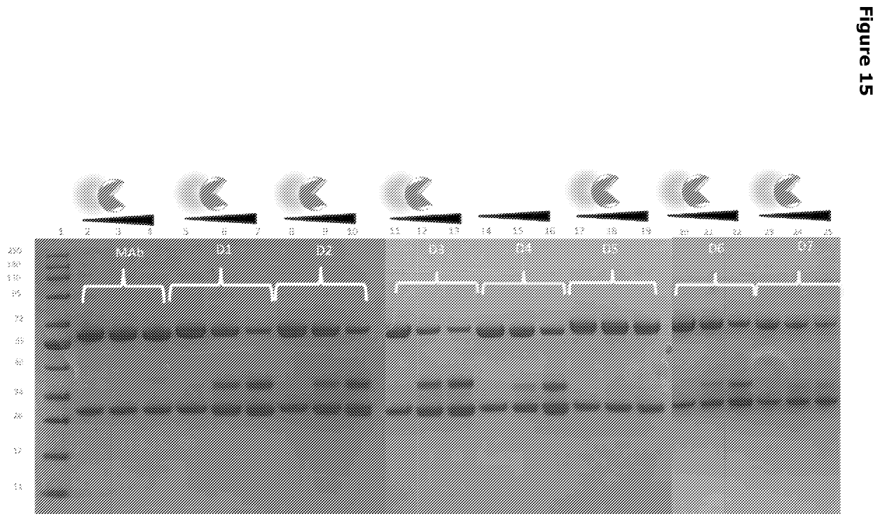

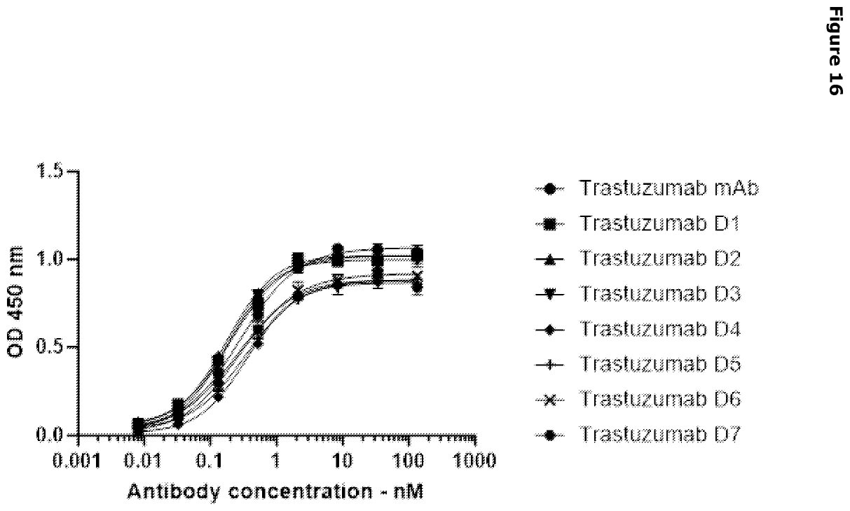

A 12-point dilution kinetics measurement of trastuzumab and D1-D7 was conducted using HBS-EP+running buffer with regeneration with 0.1 M glycine bound to hHER2-HIS with a similar KD and the samples were assayed at room temperature. All D1-D7 showed identical binding to HER2 by BIAcore (as shown in Table 6 and FIG. 14). This shows that the engineered cleavable domain has not reduced or otherwise negatively impacted the antibody's ability to bind to HER2.

TABLE 6
Binding of D1-7 and control to HER2
HER2-trastuzumab Kinetic parameters (1:1 binding)

| Trastuzumab | | | | | |
|---|---|---|---|---|---|
| variants | ka (1/Ms) | kd (1/s) | K_{D} (nM) | Rmax (RU) | Chi² (RU²) |
| D1 | 1.72E+05 | 3.24E−04 | 1.88 | 153.1 | 0.386 |
| D2 | 1.72E+05 | 3.05E−04 | 1.78 | 154.2 | 0.566 |
| D3 | 1.69E+05 | 3.14E−04 | 1.86 | 165.2 | 0.540 |
| D4 | 1.85E+05 | 3.13E−04 | 1.69 | 150.6 | 0.204 |
| D5 | 1.75E+05 | 3.10E−04 | 1.77 | 143.6 | 0.391 |
| D6 | 1.72E+05 | 3.18E−04 | 1.85 | 162.5 | 0.618 |
| D7 | 1.69E+05 | 3.67E−04 | 2.17 | 116.2 | 0.409 |
| Control | 1.63E+05 | 3.38E−04 | 2.07 | 150.3 | 0.284 |

### Protease Assay of Purified Antibodies

Conditions for the protease assay were optimised and protease buffer (50 nM Tris-HCl and 0.01% Tween-20 pH 8.0) was used to dilute the purified D1-7 antibodies and control trastuzumab. Each sample was digested with 0 nM, 23.5 nM and 235 nM of uPA enzyme and incubated at 37° C. for 24 hours. 5 μl of NuPAGE™ LDS Sample Buffer (Thermo Fisher Scientific®) loading dye with 10 nM of dithiothreitol (DDT) was added to each sample and the samples were boiled at 95° C. for 10 minutes, then cooled to 10° C. in a PCR machine. The entire contents were loaded onto a 4-12% SDS PAGE gel and run for 30 minutes at 200° C. The gel was left to stain for a few hours at room temperature in InstantBlue® stain, then left to destain in water over a few days.

It was found that the uPA protease liberates Fab or F(ab′)2 fragments from D1-D4 and D6-D7 (as shown in FIG. 15). The uPA enzyme did not digest the trastuzumab control. This experiment showed that the engineered D1-D4 and D6-D7 with protease cleavage sites were successfully cleaved, whereas the controls, D5 and trastuzumab, without protease cleavage sites, did not cleave.

Binding of uPA-Treated Cleaved Engineered Antibodies to HER2 (Assessed by ELISA)

4 ELISA plates were set up with hHER2-HIS (50 μl) at 1 μg/ml in PBS and 2 ELISA plates were set up with PBS (50 μl) to test the binding of the cleaved therapeutic domain to its binding target. The plates were covered and left at 4° C. overnight.

The plates were removed from the fridge and washed (as described above), then serially diluted 1 in 4. Each antibody was assayed in triplicate, one set each for the PBS plate, hHER2-HIS plate (Vk detection) and hHER2-HIS plate (Fc detection). The antibodies were left to incubate for 1 hour at room temperature. The plates were washed again, then the antibodies were added to the plates. The anti-Vk antibody was diluted as described above and added to 1 set of hHER2-HIS plates and the PBS plates. 50 μl was added to each well. The plates were left to incubate for 1 hour at room temperature, then washed again. The plate was developed, then the signal was quenched, and the plate was read for its absorbance at 450 nm.

It was found that uPA treatment does not affect antibody binding to hHER2 (as shown in Table 7 and FIG. 16).

TABLE 7
IC₅₀ values of uPA-treated_
trastuzumab control and D1-D7

| Antibody | IC₅₀ |
|---|---|
| Trastuzumab mAb | 0.2843 |
| Trastuzumab D1 | 0.2 |
| Trastuzumab D2 | 0.1794 |
| Trastuzumab D3 | 0.1848 |
| Trastuzumab D4 | 0.3604 |
| Trastuzumab D5 | 0.3466 |
| Trastuzumab D6 | 0.2688 |
| Trastuzumab D7 | 0.2623 |

Binding of uPA-Treated Cleaved Engineered Antibodies to Fc Gamma Receptors (Assessed by ELISA)

ELISA plates were made up to test the binding of the stabilisation domain to Fc gamma receptors (I, IIa, IIb, IIIa). Fifty μl of 1 μl/ml of Fc gamma RI (CD64), Fc gamma RIIa (CD32a), Fc gamma RIIb (CD32b), Fc gamma RIIIa (CD16a V176) and 50 μl of PBS were added to each well. The plates left at 4° C. overnight. The plates were washed, left at room temperature for 1 hour to block, then washed again.

The antibodies were tested in triplicate over an 8-point dilution with a 1 in 3 dilution factor. Fifty μl of PBS+3% dried milk was added to each well. The samples were left for 1 hour at room temperature for binding and the plates were washed. Anti-kappa IgG was diluted 1 in 5000 in PBS+3% dried milk. Samples were left to incubate for 1 hour at room temperature and the plates were washed again. The plate was developed, then the signal was quenched, and the plate was read for its absorbance at 450 nm.

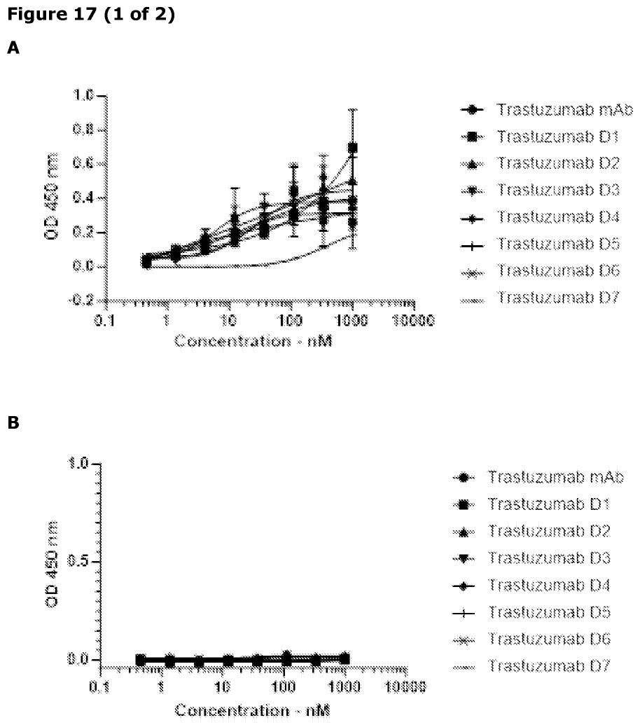

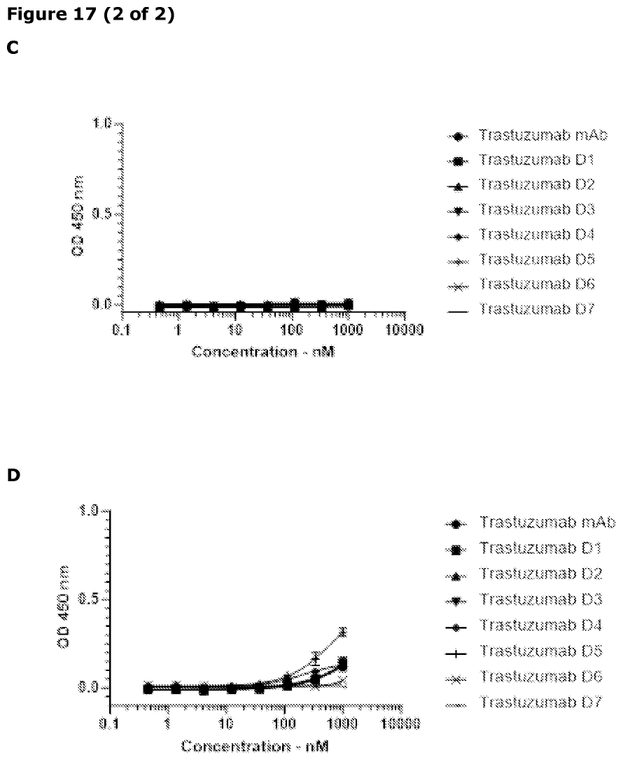

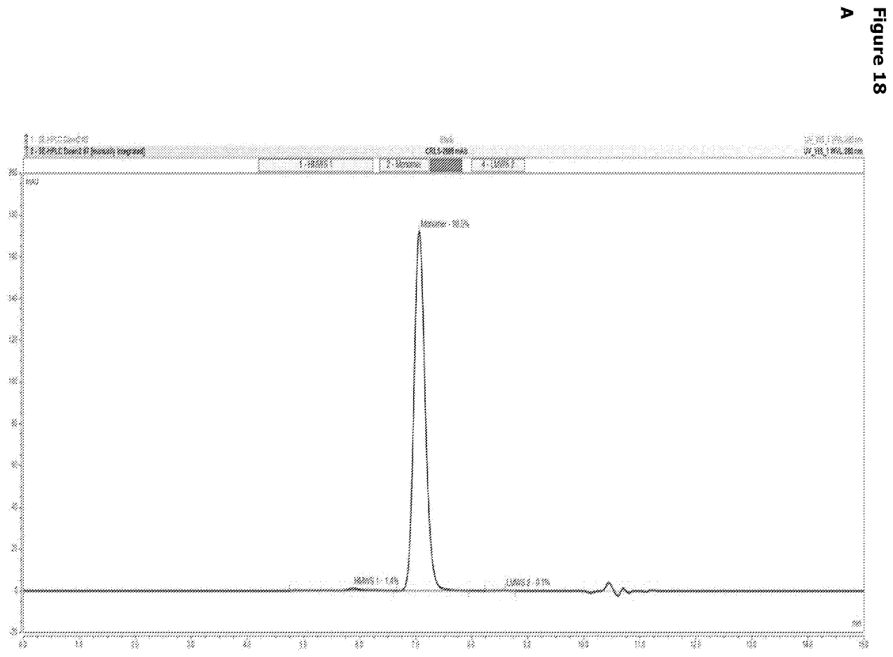

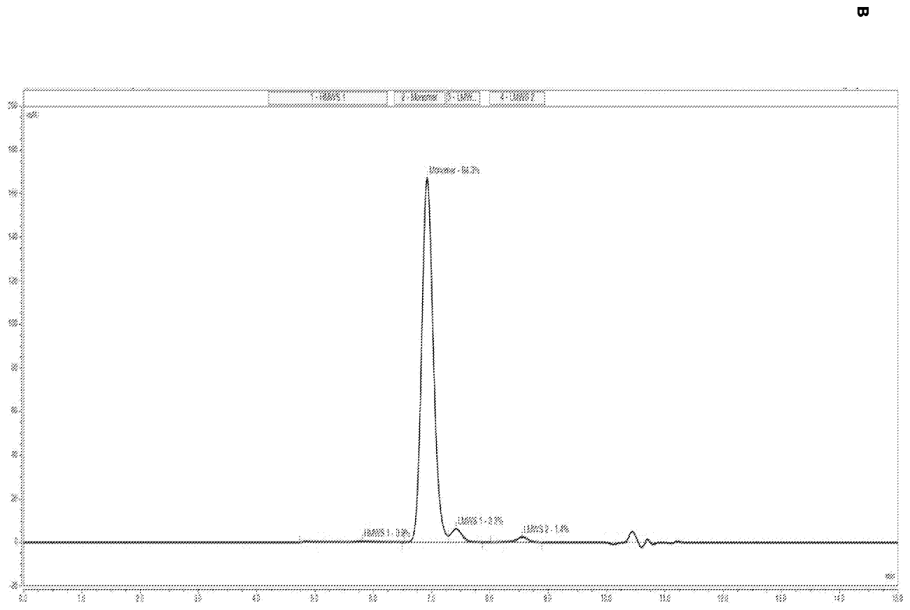

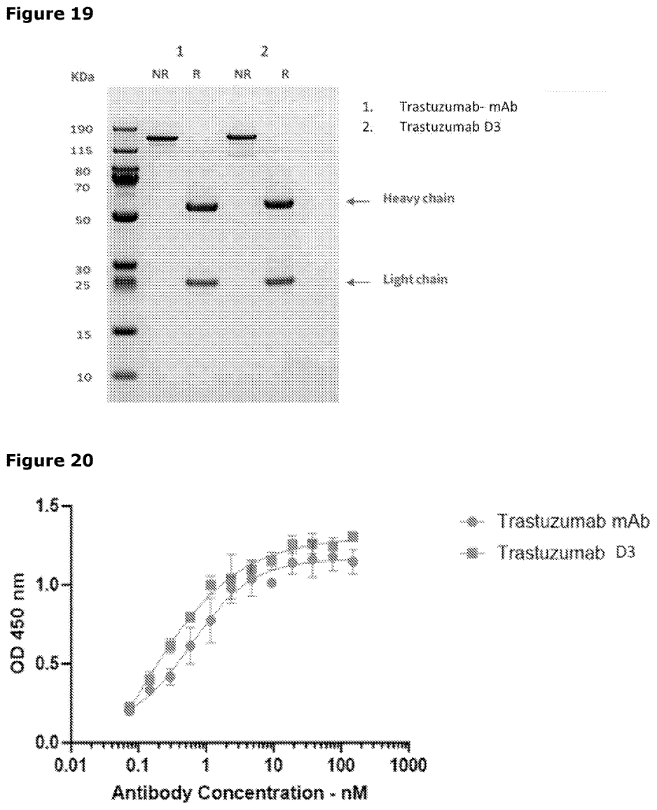

It was found that the engineered antibodies do not affect Fc gamma receptor Ia and IIa binding (as shown in FIGS. 17A and 17B). All of D1-D7 bound to Fc gamma receptor Ia strongly. D7 binding to Fc gamma receptor Ia was impacted by the protease site, which is expected to be due to the crystal structure of how the Fc region interacts with the Fc gamma receptors. There was no observed binding to Fc gamma receptor IIb (as shown in FIG. 17C). Binding to Fc gamma receptor IIIa was assessed using the 176V variant; both D6 and D7 showed a slightly decreased binding, whereas D5 showed increased binding (FIG. 17D) which is thought to be due to the glycosylation. All other engineered trastuzumab variants (D1-D4) showed similar binding to Fc gamma receptor IIIa as the trastuzumab mAb control.

### Conclusion

The engineering of a cleavable domain into an antibody-derived fragment has minimal effects of the ability of the stabilisation domain to bind to Fc gamma receptors.

### Example 5: Manufacturability of Engineered Agents

### Transfection of Trastuzumab and D3

Trastuzumab and D3 in pCREA antibody mammalian expression vectors were diluted to a concentration of 1 μg/μl and then the transfected into Expi-CHO cell lines (Invitrogen), by Optipro+ ExpiFectamine in shake flasks and incubated at 37° C. overnight. The cultures were fed and then the cells were grown for 10 days.

On day 10, the cultures were split in 500 ml conical tubes and spun at 3900×g for 30 minutes. The supernatants were then filtered through a 20 μm filter. 30 ml was transferred to a 50 ml Falcon tube and filtered, and 250 ml was purified to obtain large scale purified preparations.

### Purification of Trastuzumab and D3

The harvested trastuzumab and D3 were purified on MabSelect PrismA™ 1 mL columns using the AKTA Go Instrument (Cytiva). The relevant fractions were pooled according to chromatograms and the buffer exchanged in 1×DPBS using Vivaspin20 concentrators at 3800×g for 20 minutes. The absorbance was measured using a Nanodrop (ThermoScientific), and the concentration was determined (as shown in Table 8).

TABLE 8
Concentration of Trastuzumab and
D3 samples after Protein A purification

| | | Approximate | |
|---|---|---|---|
| | | Total | Expression |
| | Concentration | Yields | Level |
| Sample | (mg/ml) | (mg) | (mg/L) |
| Trastuzumab mAb | 6.6 | 48 | 240 |
| Trastuzumab D3 | 5.1 | 36 | 180 |

### Quality Control (QC) Analyses of Trastuzumab and D3

1 mg/ml of each sample was analysed on an isocratic run at 0.35 ml/min for 15 minutes, using a Dionex UltiMate 3000 UHPLC system (Thermo Scientific). SEC-HPLC analysis of trastuzumab showed a 98.5% monomer purity and D3 a 94.3% monomer purity (as shown in FIG. 18).

1.5 μg of each sample was analysed on a 4-12% NuPAGE SDS gel and showed good purity of D3 (as shown in FIG. 19). The samples were filtered and aliquoted into 2 ml tubes (as shown in Table 9).

TABLE 9
Final Concentration of Trastuzumab
and D3 samples after filtration

| | Concentration | Purity (% | Endotoxin |
|---|---|---|---|
| Sample | (mg/ml) | monomer) | (EU/mg/ml) |
| Trastuzumab mAb | 1.05 | 98.5 | <0.5 |
| Trastuzumab D3 | 1.04 | 94.3 | <0.5 |

### Binding of Large-Scale Preparations of Trastuzumab and D3 to Her2 (Assessed by ELISA)

An ELISA plate was prepared with 1 μg/ml of hHER2-HIS diluted in PBS, a second ELISA plate was prepared with 1 μg/ml of BSA and a third ELISA plate was prepared with PBS alone. The plates were left in the fridge overnight. The plates were then washed, left for 1 hour at room temperature to block and then washed again, as described in Example 4.

150 μl of the large-scale purified preparations (trastuzumab and D3 antibodies) were transferred into 150 μl of PBS+3% dried milk, over a 12-point dilution. 50 μl was added to each well and incubated for 1 hour at room temperature. The plates were washed, the colour was developed and the signal quenched as described above. The plate was read for absorbance at 450 nm.

Trastuzumab and D3 from large-scale expression preparations retained their ability to bind to HER2. Both trastuzumab and D3 were found to be active. Trastuzumab bound hHER2 with an IC₅₀ of 0.6095 nM and D3 bound hHER2 with an IC₅₀ of 0.1035 nM (as shown in Table 10 and FIG. 20).

TABLE 10
IC₅₀ values of Trastuzumab and D3 from ELISA

| | Trastuzumab mAb | Trastuzumab D3 |
|---|---|---|
| IC₅₀ (nM) | 0.6095 | 0.1035 |

### Purification of Trastuzumab and D1

The trastuzumab control and D1 were expressed from the transfected CHO-K1 cells, as described in Example 3. The samples were spun down at 3900×g for 20 minutes and filtered through a 0.22 μm filter, then loaded onto a MabSelect PrismA 1 ml prepacked column and purified using the AKTA Go Instrument (Cytiva). The relevant fractions were pooled, and buffer was exchanged in DPBS using a Vivaspin20, 30 kDa MWCO and spinning in a benchtop centrifuge at 3800×g for 20 minutes. The absorbance was measured using a Nanodrop (ThermoScientific), and the yield and concentration was calculated. It was found that expression levels for both trastuzumab and D1 were very low, likely due to a plasmid/cell line lack of optimisation (as shown in Table 11). However, the expression levels between the two were comparable showing the engineered agent maintains the purity of trastuzumab.

TABLE 11
Expression levels of
CHO-K1 Trastuzumab and D1

| | Expression |
|---|---|
| Sample | Levels (mg/L) |
| Trastuzumab | 6.5 |
| (CHO-K1) | |
| D1 | 5.5 |

### SDS-PAGE Analysis of Purified Trastuzumab mAb and D1

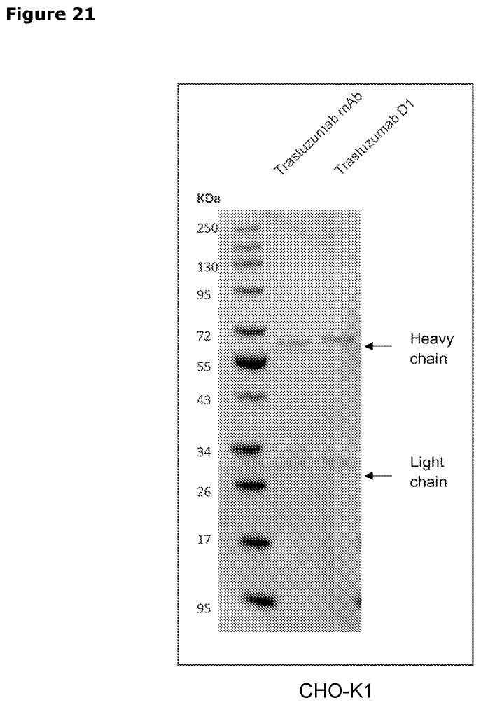

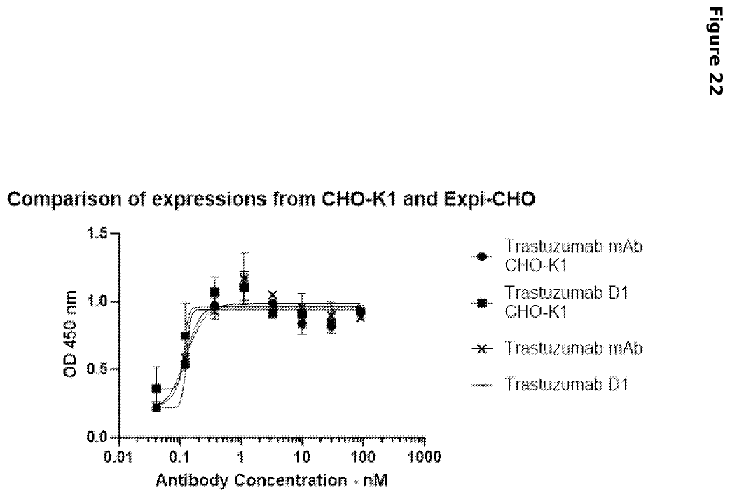

The purified trastuzumab and D1 samples were boiled at 85° C. for 5 minutes, then separated by SDS-PAGE. The gel showed intact antibody for both trastuzumab and D1 (as shown in FIG. 21).

Binding of Trastuzumab and D1 Expressed from Expi-CHO and CHO-K1 Cells to HER2 (Assessed by ELISA)

Three Maxisorb ELISA plates for each antigen were prepared by adding either 50 μl of PBS, 1 μg/ml of BSA or 1 μg/ml of hHER2 in each well and placed into the fridge overnight. The plates were washed and left to block at room temperature for 1 hour, then washed again. The antibody samples were purified according to Examples 3 and 6 above, were diluted 1 in 3 to a final volume of 50 μl per well, and each antibody was assayed in triplicate over an 8-point dilution. The samples were diluted in PBS+3% dried milk and incubated for 1 hour at room temperature. The plates were prepared with the anti-Vk antibody as described above. The samples were left to bind at room temperature for 1 hour. The plates were washed as described above. The colour was developed, the signal quenched and the plate was read for absorbance at 450 nm.

Trastuzumab and D1 expressed from Expi-CHO or CHO-K1 cells were identical as judged by ELISA and the potency of trastuzumab was not compromised by insertion of an exogenous cleavable domain (as shown in FIG. 22 and Table 12).

TABLE 12
IC₅₀ values of expression of trastuzumab
and D1 from CHO-K1 and Expi-CHO

| Antibodies | IC₅₀ |
|---|---|
| Trastuzumab mAb (CHO-K1) | 0.1262 |
| Trastuzumab D1 (CHO-K1) | 0.1171 |
| Trastuzumab mAb (Expi-CHO) | 0.1251 |
| Trastuzumab D1 (Expi-CHO) | 0.1270 |

### Example 6: Different Protease Cleavage Sites are Amenable to the Engineered Agent Design

To demonstrate that this technology can be used with different types of cancer specific protease cleavage sites, different agents were designed with an ADAM10 site (D9). This protease was selected because it belongs to different protease family than uPA (which is a serine proteases). ADAM10 is a metalloprotease Napsin-A. The designs were based on the engineered trastuzumab antibody design 3.

### Cloning

The constructs incorporating the different protease cleavage site (ADAM10 site: PRAEALKGG (SEQ ID NO: 89, underlined) was designed and expressed. The constructs were based on the engineered trastuzumab antibody design 3.

Engineered Antibody with ADAM10 Site

In the engineered trastuzumab antibody design 9 (D9) GSPRAEALKGGGSAS (SEQ ID NO: 90) was added after the cysteine residue that ends the CH1 domain and prior to the DKTHT (SEQ ID NO: 78) hinge region (italicised). This adds an additional 2 amino acids on the C and N terminal end and an additional 4 amino acids on the N terminal end to the protease cleavage site for greater accessibility.

Engineered trastuzumab antibody design 9, D9
(SEQ ID NO: 8):
EVQLVESGGGLVQPGGSLRLSCAASGFNIKDTYIHWVRQAPGKGLEWVAR
IYPTNGYTRYADSVKGRFTISADTSKNTAYLQMNSLRAEDTAVYYCSRWG
GDGFYAMDYWGQGTLVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKKVEPKSCGSPRAEALKGGGSASDKTHTCPPCPAP
ELLGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPAPI
EKTISKAKGQPREPQVYTLPPSREEMTKNQVSLTCLVKGFYPSDIAVEWE
SNGQPENNYKTTPPVLDSDGSFFLYSKLTVDKSRWQQGNVFSCSVMHEAL
HNHYTQKSLSLSPGK
A geneblock of D9 was synthesised (Integrated
DNA Technologies), as given below:
(SEQ ID NO: 91)
attaagctcttccctggccGAGGTTCAACTGGTTGAGAGTGGAGGGGGAT
TGGTGCAGCCAGGCGGCAGTCTGCGGTTGAGCTGCGCAGCCTCTGGATTC
AATATCAAAGATACTTACATACACTGGGTCCGACAGGCACCTGGCAAGGG
GCTTGAGTGGGTTGCCCGTATTTATCCCACTAACGGTTATACACGGTACG
CCGACTCTGTTAAAGGTCGATTCACAATAAGTGCAGATACCTCCAAAAAC
ACAGCTTATCTGCAAATGAACAGCCTTCGCGCAGAAGATACTGCAGTTTA
TTATTGCTCACGCTGGGGTGGGGATGGCTTCTACGCTATGGACTATTGGG
GGCAGGGCACCCTGGTCACTGTATCCTCTGCCTCTACAAAAGGTCCATCC
GTATTCCCATTGGCTCCAAGCTCTAAGAGTACTTCTGGTGGTACAGCCGC
CCTTGGATGTCTCGTAAAAGATTACTTCCCTGAACCTGTGACCGTCTCCT
GGAATTCAGGGGCACTGACAAGCGGCGTTCATACTTTTCCTGCCGTTCTT
CAGTCAAGTGGCCTTTACAGTCTGAGTTCCGTCGTAACAGTCCCTAGCTC
AAGTCTTGGGACACAGACTTATATCTGCAACGTAAATCACAAACCCTCTA
ATACTAAGGTAGACAAGAAAGTGGAGCCCAAATCCTGTGGGTCTCCACGA
GGCTGGGATGCTCATACTGGGTCTgctggaagagcgg

100 ng of each geneblock was mixed with 100 ng of pCREA high throughput mammalian Fc vectors along with 5 units of SapI restriction enzyme (New England Biolabs) and 200 units of T4 DNA ligase (New England Biolabs). Ligations were transformed into NEB 5 alpha cells (New England Biolabs) and plated on LB agar plates containing 100 μg/ml of ampicillin. Plates were incubated at 37° C. overnight. 3 colonies were checked for the presence of the correct insert by mini prep followed by Sanger sequencing. For large scale preparation of DNA for transfection, the correct constructs were prepped using the endotoxin free maxi prep kit (Qiagen).

### Purification of D9

Prior to purification, D9 was produced by transfecting Expi-CHO cells, wherein expression was carried out at a 25 ml scale for 7-10 days. Purification buffers were filtered through a 20 μm filter. The supernatant was loaded on MabSelect PrismA resin and samples were run on the AKTA Go instrument. The relevant fractions were pooled and buffer exchanged in 1×PBS pH 7.2 using a Vivaspin20, 30 kDa MWCO spinning in a benchtop centrifuge at 3800×g for 20 minutes. The absorbance was measured using a Nanodrop (ThermoScientific) and the yield and concentration were calculated (see Table 14 below).

TABLE 14
Yield and Concentration of D9

| Sample | A280 | Ext. Coeff. | Conc (mg/ml) | Vol (ml) | Yield (mg) |
|---|---|---|---|---|---|
| D9 | 4.97 | 1.441 | 3.4 | 0.7 | 2.4 |

### SDS-PAGE Analysis of D9

The samples were boiled at 85° C. for 5 minutes, then 10 μl of each were loaded on the precast NuPAGE 4-12% Bis-Tris Gel inserted in a mini Gel Tank filled with 1×MES SDS running buffer, with a protein marker. The gel was run for 25 minutes at 200 V. The gel was then stained using InstantBlue® protein stain and was subsequently destained with H₂O.

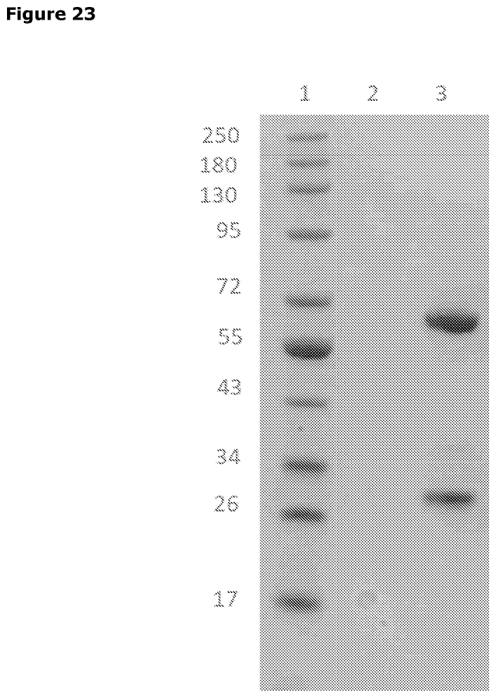

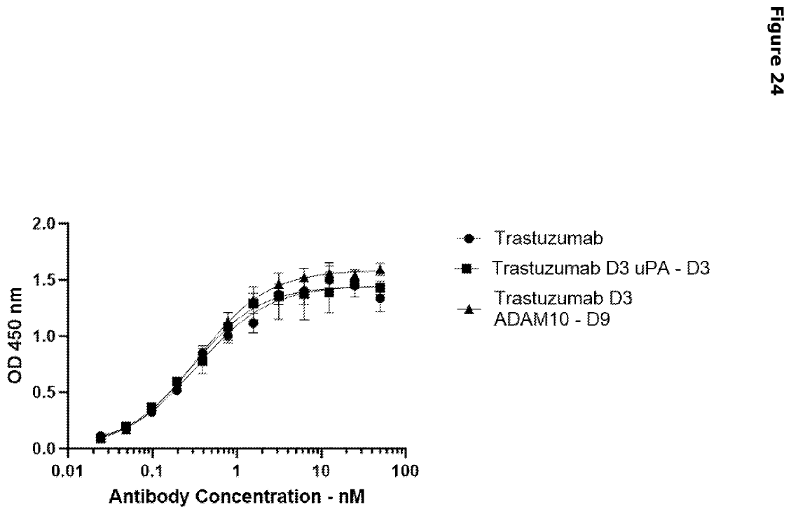

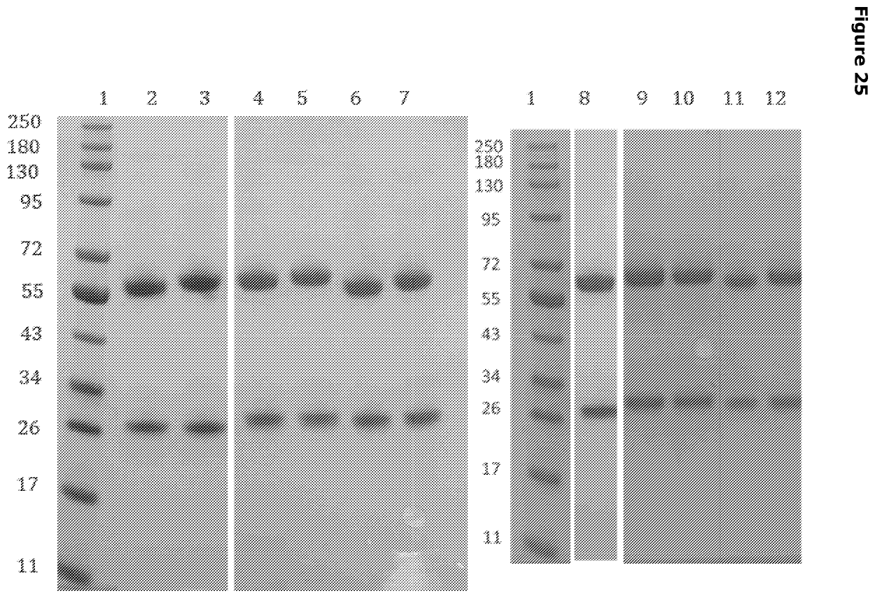

All molecules looked as expected on an SDS-PAGE, and the D9 showed a comparable band size and purity to the other samples (as shown in FIG. 23).

These results show that replacing the uPA cleavage site with another cleavage site does not impact the expression of the trastuzumab engineered antibodies in the D3 position.

### Protease Digestions

The protease ADAM10 was diluted 1:10, and the digestion was set up so that 5 μg of D9 was digested with a single concentration of protease at a 1:1 molar ratio of antibody to protease. The sample was prepared in 1.5 ml Eppendorf tubes and incubated at 37° C. for 24 hours.

The cleaved engineered antibodies were boiled at 85° C. for 5 minutes, then separated by SDS-PAGE.

D9 was efficiently cut by ADAM10 (see FIG. 23), and thus all designs are amenable to be cleaved by their respective protease.

Binding of D9 to hHER2 (Assessed by ELISA)

Plates were set up as follows: 1 plate of hHER2-HIS diluted to 1 ug/ml, 1 plate of BSA diluted to 1 μg/ml, and 1 plates of PBS blank for D3 (control) and D9. 50 μl of each antigen was added to each well and were diluted in PBS. The plates were washed, then left for 1 hour at room temperature to block, before being washed again.

150 μl of trastuzumab, D3 and D9 was transferred into 150 μl of PBS+3% dried milk making a 1 in 2 dilution. 50 μl was added to each well and the proteins were incubated for 1 hour at room temperature. The plates were prepared with the anti-Vk antibody and samples as described above. The colour was developed, the signal quenched and the plate was read for absorbance at 450 nm.

D9 was found to retain potency and binding towards HER2 (see FIG. 24 and Table 20).

TABLE 20
IC₅₀ values of trastuzumab control, D3 and D9

| Antibody | IC₅₀ |
|---|---|
| Trastuzumab mAb | 0.3363 |
| Trastuzumab D9 | 0.3389 |

This example shows that cancer-specific protease sites from a diverse protease families can be amenable to this technology.

### Example 7: Different Antibody-Derived Fragments are Amenable to the Engineered Agent Design

To demonstrate that this technology can be used with different types of antibody-derived fragments, different agents were designed with fragments derived from pembrolizumab, cetuximab, bevacizumab, M5A and ipilimumab. These specific antibody-derived fragments were selected because they each bind to a different target and a used to treat diverse types of cancers. The designs were based on the engineered trastuzumab antibody design 3.

### Cloning

Other clinical antibodies relevant in oncology were assessed for their amenability for hinge engineering. The antibodies ipilimumab (as described in US20150283234), cetuximab, pembrolizumab, bevacizumab and an anti-CEA antibody (M5A as described in U.S. Pat. No. 7,273,608 B2; Yazaki et al, 2004. Humanization of the anti-CEA T84.66 antibody based on crystal structure data. Protein Engineering, Design and Selection. 17(5): 481-489) were selected. The designs, were based on the engineered trastuzumab antibody D3, which adds GS-[protease site]-GS after the cysteine in CH1 (upper hinge). The cleavable domain is shown in bold. The uPA cleavage site is underlined.

The VRC01 antibody was used as a negative control because the target of VRC01 is a non-mammalian antigen, namely the human immunodeficiency virus (HIV).

VRC01 control design
(SEQ ID NO: 92)
QVRLVQSGPQIKTPGASVTISCGTSGYDFMESLINWVRQDIGKGPEWMGW
INPRGGGVNYGRRFQGKVTMTRDVSSGTAYLTLRGLTSDDTAKYYCVRGK
SCCGGRRYCNGADCFNWDFEHWGQGTLVIVSSASTKGPSVFPLAPSSKST
SGGTAALGCLVKDYFPEPVTVSWNSGALTSGVHTFPAVLQSSGLYSLSSV
VTVPSSSLGTQTYICNVNHKPSNTKVDKKVEPKSCGSLSGRSDNHGSASD
KTHTCPPCPAPELLGGPSVFLFPPKPKDTLMISRTPEVTCVVVDVSHEDP
EVKFNWYVDGVEVHNAKTKPREEQYNSTYRVVSVLTVLHQDWLNGKEYKC
KVSNKALPAPIEKTISKAKGQPREPQVYTLPPSREEMTKNQVSLTCLVKG
FYPSDIAVEWESNGQPENNYKTTPPVLDSDGSFFLYSKLTVDKSRWQQGN
VFSCSVMHEALHNHYTQKSLSLSPGK
Cetuximab D12
(SEQ ID NO: 11)
QVQLKQSGPGLVQPSQSLSITCTVSGFSLTNYGVHWVRQSPGKGLEWLGV
IWSGGNTDYNTPFTSRLSINKDNSKSQVFFKMNSLQSNDTAIYYCARALT
YYDYEFAYWGQGTLVTVSAASTKGPSVFPLAPSSKSTSGGTAALGCLVKD
YFPEPVTVSWNSGALTSGVHTFPAVLQSSGLYSLSSVVTVPSSSLGTQTY
ICNVNHKPSNTKVDKKVEPKSCGSLSGRSDNHGSASDKTHTCPPCPAPEL
LGGPSVFLFPPKPKDTLMISRTPEVTCVVVDVSHEDPEVKFNWYVDGVEV
HNAKTKPREEQYNSTYRVVSVLTVLHQDWLNGKEYKCKVSNKALPAPIEK
TISKAKGQPREPQVYTLPPSREEMTKNQVSLTCLVKGFYPSDIAVEWESN
GQPENNYKTTPPVLDSDGSFFLYSKLTVDKSRWQQGNVFSCSVMHEALHN
HYTQKSLSLSPGK
Bevacizumab D13
(SEQ ID NO: 12)
EVQLVESGGGLVQPGGSLRLSCAASGYTFTNYGMNWVRQAPGKGLEWVGW
INTYTGEPTYAADFKRRFTFSLDTSKSTAYLQMNSLRAEDTAVYYCAKYP
HYYGSSHWYFDVWGQGTLVTVSSASTKGPSVFPLAPSSKSTSGGTAALGC
LVKDYFPEPVTVSWNSGALTSGVHTFPAVLQSSGLYSLSSVVTVPSSSLG
TQTYICNVNHKPSNTKVDKKVEPKSCGSLSGRSDNHGSASDKTHTCPPCP
APELLGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALPA
PIEKTISKAKGQPREPQVYTLPPSREEMTKNQVSLTCLVKGFYPSDIAVE
WESNGQPENNYKTTPPVLDSDGSFFLYSKLTVDKSRWQQGNVFSCSVMHE
ALHNHYTQKSLSLSPGK
Pembrolizumab D14a
(SEQ ID NO: 13)
QVQLVQSGVEVKKPGASVKVSCKASGYTFTNYYMYWVRQAPGQGLEWMGG
INPSNGGTNFNEKFKNRVTLTTDSSTTTAYMELKSLQFDDTAVYYCARRD
YRFDMGFDYWGQGTTVTVSSASTKGPSVFPLAPCSRSTSESTAALGCLVK
DYFPEPVTVSWNSGALTSGVHTFPAVLQSSGLYSLSSVVTVPSSSLGTKT
YTCNVDHKPSNTKVDKRVESKYGSLSGRSDNHGSGPPCPPCPAPEFLGGP
SVFLFPPKPKDTLMISRTPEVTCVVVDVSQEDPEVQFNWYVDGVEVHNAK
TKPREEQFNSTYRVVSVLTVLHQDWLNGKEYKCKVSNKGLPSSIEKTISK
AKGQPREPQVYTLPPSQEEMTKNQVSLTCLVKGFYPSDIAVEWESNGQPE
NNYKTTPPVLDSDGSFFLYSRLTVDKSRWQEGNVFSCSVMHEALHNHYTQ
KSLSLSLGK
Pembrolizumab D14b
(SEQ ID NO: 14)
QVQLVQSGVEVKKPGASVKVSCKASGYTFTNYYMYWVRQAPGQGLEWMGG
INPSNGGTNFNEKFKNRVTLTTDSSTTTAYMELKSLQFDDTAVYYCARRD
YRFDMGFDYWGQGTTVTVSSASTKGPSVFPLAPCSRSTSESTAALGCLVK
DYFPEPVTVSWNSGALTSGVHTFPAVLQSSGLYSLSSVVTVPSSSLGTKT
YTCNVDHKPSNTKVDKRVESKYGPPGSLSGRSDNHGSCPPCPAPEFLGGP
SVFLFPPKPKDTLMISRTPEVTCVVVDVSQEDPEVQFNWYVDGVEVHNAK
TKPREEQFNSTYRVVSVLTVLHQDWLNGKEYKCKVSNKGLPSSIEKTISK
AKGQPREPQVYTLPPSQEEMTKNQVSLTCLVKGFYPSDIAVEWESNGQPE
NNYKTTPPVLDSDGSFFLYSRLTVDKSRWQEGNVFSCSVMHEALHNHYTQ
KSLSLSLGK

In the engineered trastuzumab antibody designs 14 (D14a and D14b) an additional 3 residues GPP (italicised) were either added either to the C-terminal of the cleavage site (D14a) or to the N-terminal of the cleavage site within the cleavable domain. This helped with the expression and purification of the engineered antibody.

M5A D15
(SEQ ID NO: 15)
EVQLVESGGGLVQPGGSLRLSCAASGFNIKDTYMHWVRQAPGKGLEWVAR
IDPANGNSKYADSVKGRFTISADTSKNTAYLQMNSLRAEDTAVYYCAPFG
YYVSDYAMAYWGQGTLVTVSSASTKGPSVFPLAPSSKSTSGGTAALGCLV
KDYFPEPVTVSWNSGALTSGVHTFPAVLQSSGLYSLSSVVTVPSSSLGTQ
TYICNVNHKPSNTKVDKKVEPKSCGSLSGRSDNHGSASDKTHTCPPCPAP
ELLGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPAPI
EKTISKAKGQPREPQVYTLPPSREEMTKNQVSLTCLVKGFYPSDIAVEWE
SNGQPENNYKTTPPVLDSDGSFFLYSKLTVDKSRWQQGNVFSCSVMHEAL
HNHYTQKSLSLSPGK
Ipilimumab D16
(SEQ ID NO: 16)
QVQLVESGGGVVQPGRSLRLSCAASGFTFSSYTMHWVRQAPGKGLEWVTF
ISYDGNNKYYADSVKGRFTISRDNSKNTLYLQMNSLRAEDTAIYYCARTG
WLGPFDYWGQGTLVTVSSASTKGPSVFPLAPSSKSTSGGTAALGCLVKDY
FPEPVTVSWNSGALTSGVHTFPAVLQSSGLYSLSSVVTVPSSSLGTQTYI
CNVNHKPSNTKVDKRVEPKSCGSLSGRSDNHGSASDKTHTCPPCPAPELL
GGPSVFLFPPKPKDTLMISRTPEVTCVVVDVSHEDPEVKFNWYVDGVEVH
NAKTKPREEQYNSTYRVVSVLTVLHQDWLNGKEYKCKVSNKALPAPIEKT
ISKAKGQPREPQVYTLPPSREEMTKNQVSLTCLVKGFYPSDIAVEWESNG
QPENNYKTTPPVLDSDGSFFLYSKLTVDKSRWQQGNVFSCSVMHEALHNH
YTQKSLSLSPGK

### Transfection

All plasmids (vector: pCrea) intended for transfection were diluted to a concentration of 1 μg/μl and then the transfected into Expi-CHO cell lines (Invitrogen), by Optipro+ ExpiFectamine in shake flasks and incubated at 37° C. overnight. The cultures were fed and then the cells were grown for 11 days. After which, the cultures were spun at 3900×g for 30 minutes, then the supernatants were the filtered through a 20 μm filter, then stored at 4° C.

### Purification and Buffer Exchange

Purification buffers were prepared then filtered through a 20 μm filter. The cell culture supernatants were purified using MabSelect PrismA (Cytiva) and formulated in 1×PBS pH 7.2, as described above. The relevant fractions were pooled and buffer exchanged in DPBS using a Vivaspin20, 30 kDa MWCO spinning in a benchtop centrifuge at 3800×g for around 60 minutes, as described above. The buffer exchanged molecules were sterile-filtered through a 20 μm filter.

TABLE 15
Expression levels of different antibodies
expressed with and without cleavable domain

| | | | | Expression |
|---|---|---|---|---|
| | Volume | Concentration | Yield | Level |
| Construct | (ml) | (mg/ml) | (mg) | (mg/L) |
| Bevacizumab | 2.5 | 4.55 | 11.39 | 379.1 |
| D13 | 2.5 | 4.94 | 12.35 | 418.7 |
| Pembrolizumab | 2.5 | 0.7 | 1.8 | 72.9 |
| D14a | 2.5 | 0.73 | 1.8 | 72.9 |
| D14b | 3.0 | 0.28 | 0.8 | 33.4 |
| Cetuximab | 3.5 | 2.61 | 9.13 | 337.7 |
| D12 | 2.3 | 3.93 | 9.05 | 341.0 |
| M5A | 3.0 | 2.57 | 7.70 | 310.6 |
| D15 | 2.5 | 2.81 | 7.02 | 286.6 |
| Ipilumimab | 3.5 | 1.2 | 4.1 | 165.8 |
| D16 | 3.0 | 3.6 | 10.7 | 427.2 |

### SDS-PAGE Analysis

1 μg aliquots of samples were prepared according to Table 16.

TABLE 16
Purified antibody preparation for SDS-PAGE analysis

| | | mAb | engineered | engineered |
|---|---|---|---|---|
| μl | mAb R | Non-R | Ab R | Ab Non-R |
| Sample | 2.3 | 2.3 | 2.8 | 2.8 |
| H₂O | 11.7 | 12.7 | 11.2 | 12.2 |
| SDS Loading | 5 | 5 | 5 | 5 |
| Buffer (4X) | | | | |
| 1M DTT (20X) | 1 | 0 | 1 | 0 |
| Total volume | 20 | 20 | 20 | 20 |

The purified antibodies were boiled at 85° C. for 5 minutes, 10 μl of each was loaded on a NuPAGE 4-12%/Bis-Tris Gel inserted in a mini Gel Tank filled with 1×MES SDS running buffer, with a protein marker. The gel was run for 25 minutes at 200V, then stained using InstantBlue® protein stain and was subsequently destained with H₂O, as described above.

Both the reduced and non-reduced SDS PAGE showed expected expression and purity profiles for all loaded antibodies (FIG. 25).

### Protease Digestions of Antibody Variants

The protocol described above (see Example 4 and 6) was used to test whether the variants of the other clinical antibodies relevant in oncology prepared as described above were amenable to digestion.

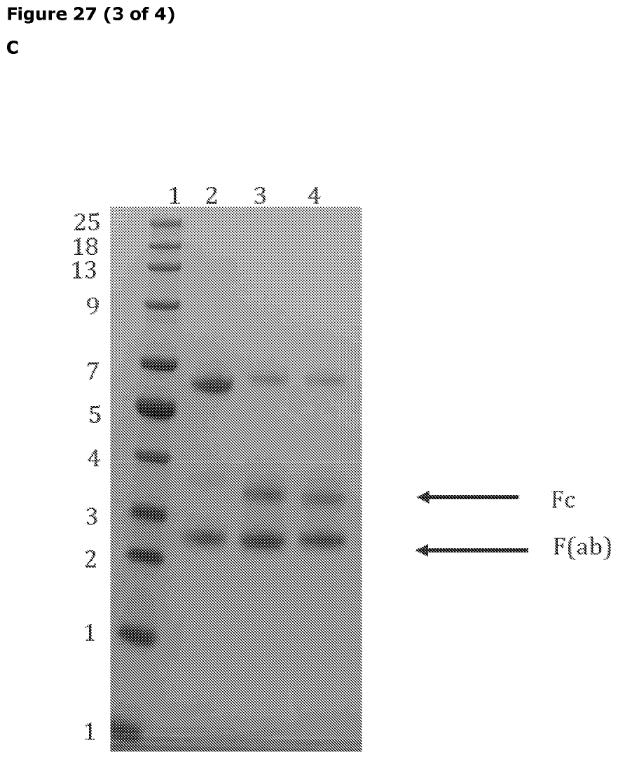

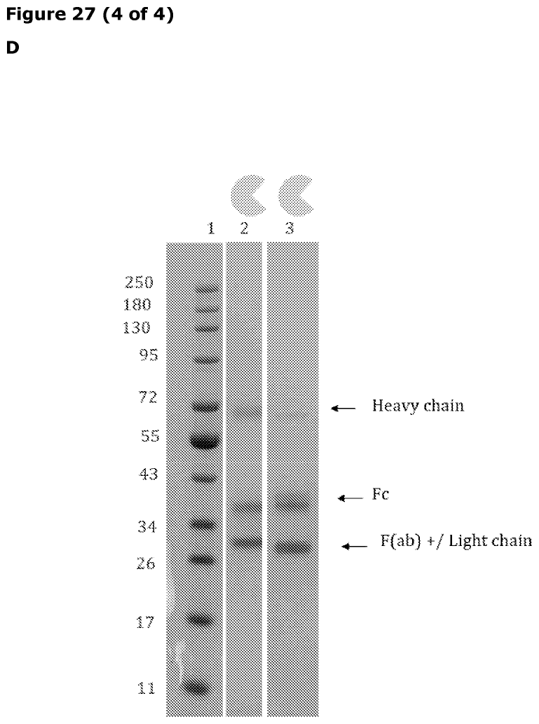

It was found that uPA cleaved the antibody variants and generated F(ab) fragments in a similar manner as it did for Trastuzumab D3 (as shown in FIGS. 27A, 27B, 27C and 27D).

Binding of D12-D16 to their Respective Targets (Assessed by ELISA)

ELISA plates were set up according to Table 17.

TABLE 17
ELISA plate set up for D12-D16

| Number | | |
|---|---|---|
| of plates | Reagent | Dilution |
| 1 | hEGFR-HIS | 1 μg/ml |
| 1 | hVEGF-HIS | 1 μg/ml |
| 1 | hPD1-HIS | 1 μg/ml |
| 1 | hCEACAM-5 | 1 μg/ml |
| 1 | hCTLA-4 | 1 μg/ml |
| 2 | BSA | 1 μg/ml |
| 2 | PBS | Blank |

50 μl was added to each well. All antigens were diluted in PBS. The plates, anti Vk antibody and samples were prepared as described above. The colour was developed, the signal sequenced and the plate read for absorbance at 450 nm.

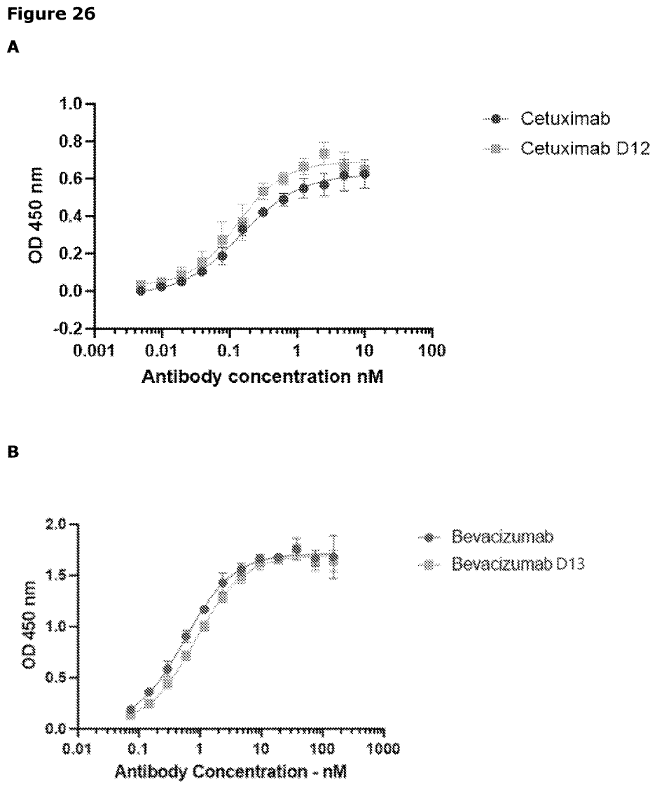

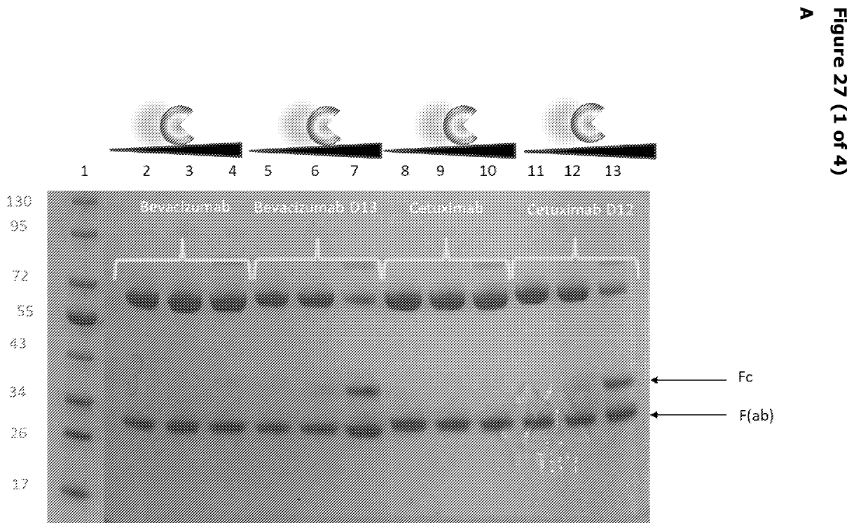

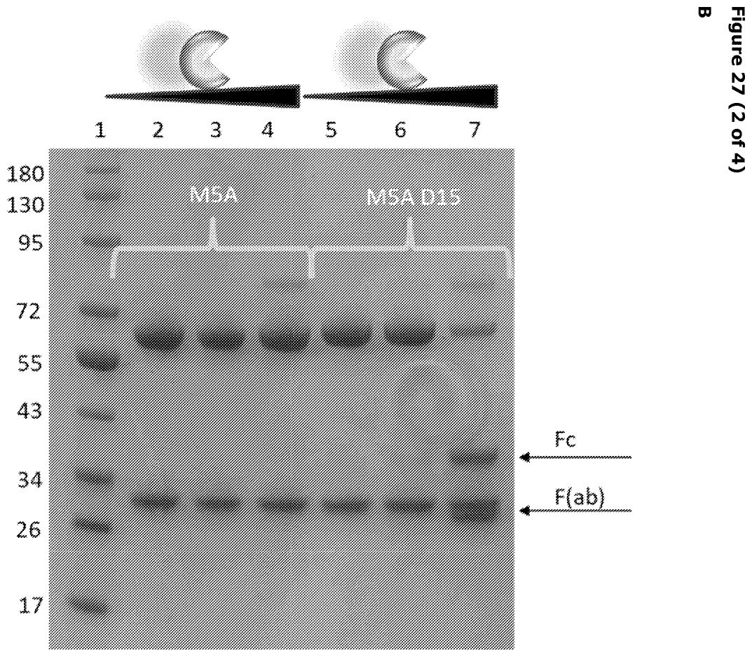

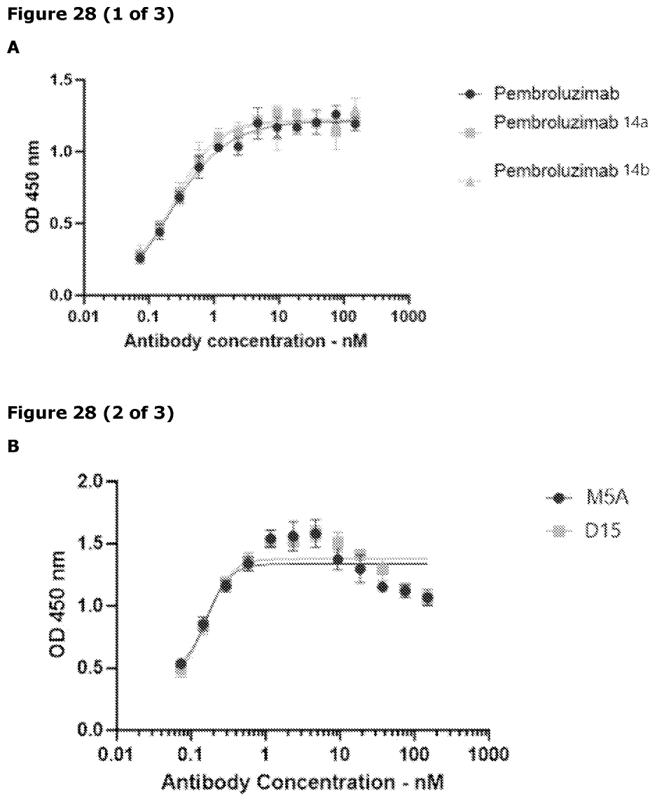

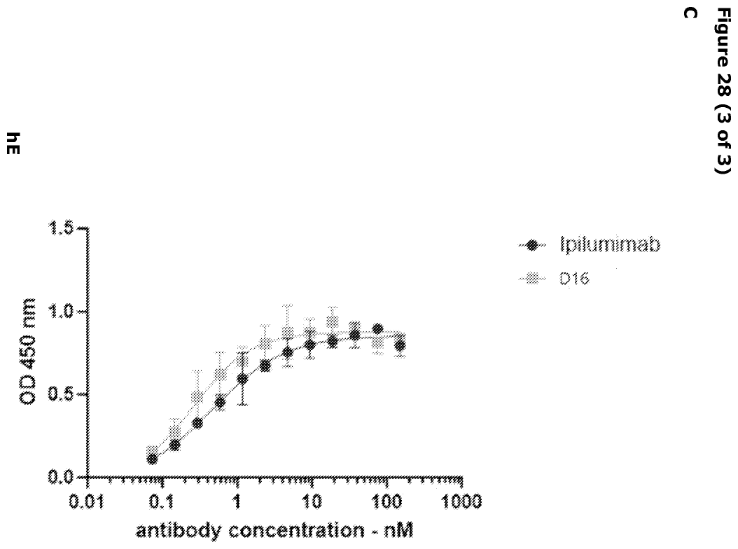

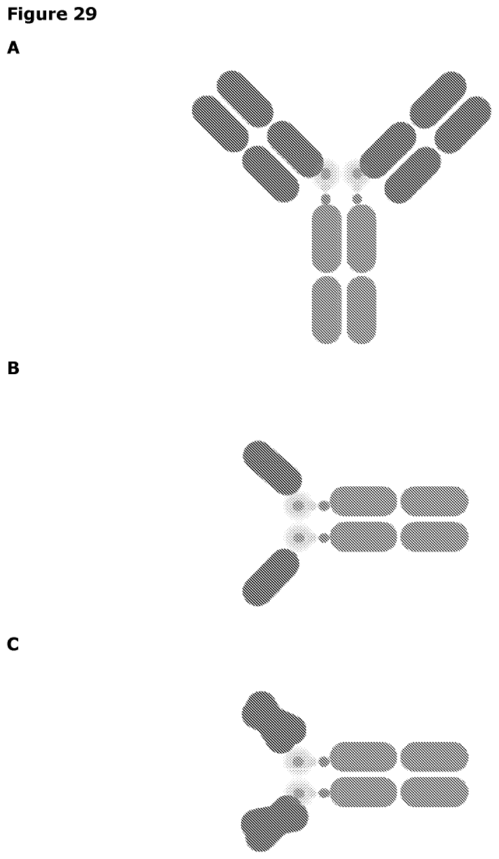

All of D12-D16 retained similar binding profiles to their respective targets (hEGFR, hVEGF, hPD-1, hCEACAM-5 and hCTLA-4) as compared to the non-engineered antibodies (as shown in FIGS. 26A, 26B, 28A, 28B and 28C, and Table 18).

TABLE 18
IC₅₀ values for non-engineered and engineered antibodies D13-D16

| | | Engineered | |
|---|---|---|---|
| WT mAb | IC₅₀ | antibody | IC₅₀ |
| Cetuximab | 0.1486 | D12 | 0.13 |
| Bevacizumab | 0.5640 | D13 | 0.8450 |
| Pembrolizumab | 0.1781 | D14a | 0.2428 |
| | | D14b | 0.2885 |
| M5A | 0.1603 | D15 | 0.1584 |
| Ipilimumab | 0.4610 | D16 | 0.2645 |

D12-D16 were amenable to cleavage and found to retain potency and binding towards their respective targets.

This example shows that different antibody-derived fragments which bind to a diverse range of targets (and whose mechanism of action in cancer may differ) can be amenable to this technology.

### Example 9: Different Types of Therapeutic Domains are Amenable to the Engineered Agent Design

To demonstrate that this technology can be used with different types of therapeutic domains, different agents were designed. In anti-EGFR VHH design 17 (D17), an anti-EGFR V_{H}H (llama 7D12) domain was used as a therapeutic domain. In Trastuzumab designs 18 (D18) and 19 (D19), a ScFv domain was used as therapeutic domain. D19 differs from D18 in that the stabilisation domain contains a longer linker. The designs were based on the engineered trastuzumab antibody design 3.

### Cloning

### VHH as a Therapeutic Domain

The llama 7D12 VHH for an anti-EGFR antibody sequence was taken from the crystal structure of EGFR with VHH nanobodies from Schmitz K R, Bagchi A, Roovers R C, van Bergen en Henegouwen P M, Ferguson K M. Structural evaluation of EGFR inhibition mechanisms for nanobodies/VHH domains. Structure. 2013 Jul. 2; 21(7):1214-24. doi: 10.1016/j.str.2013.05.008. Epub 2013 Jun. 20. PMID: 23791944; PMCID: PMC3733345.

Geneblock of anti-EGFR VHH design 17 (D17)
(SEQ ID NO: 93)
attaagctcttccctggccCAAGTCCAACTGCAGGAGAGCGGGGGTGGCC
TGGTACAGCCAGGAGGTAGCCTTCGGCTGTCATGTGCAGCCAGCGGACGC
ACATTTTCCAGTTACGCAATGGGTTGGTTTAGGCAAGCCCCTGGTAAACA
GAGAGAATTCGTCGCTGCCATTCGGTGGAGTGGAGGTTATACATACTACA
CAGATAGTGTTAAGGGTCGTTTTACTATTTCTCGGGACAATGCTAAAACC
ACAGTTTATCTCCAGATGAATAGTTTGAAGCCCGAGGACACAGCCGTATA
TTATTGTGCCGCCACTTATCTTTCTAGCGACTACTCCCGTTACGCCTTGC
CACAACGTCCTCTTGACTATGACTATTGGGGACAAGGTACTCAAGTAACC
GTTTCCTCATCTCTCTCCGGTCGATCCGACAACCACGGGTCTgctggaag
agcgg

The ScFvs and VHH were cloned into the pET49b+ based vector for Escherichia coli expression using restriction sites NdeI and PacI (New England Biolabs). They also contained a PeIB or DSB leader sequence to allow for export of the ScFv and VHH into the cell supernatant.

The design was based on the engineered trastuzumab antibody D3. The cleavable domain is shown in bold, and the cleavage site is underlined.

anti-EGFR VHH D17
(SEQ ID NO: 9)
QVQLQESGGGLVQPGGSLRLSCAASGRTFSSYAMGWFRQAPGKQREFVAA
IRWSGGYTYYTDSVKGRFTISRDNAKTTVYLQMNSLKPEDTAVYYCAATY
LSSDYSRYALPQRPLDYDYWGQGTQVTVSSSLSGRSDNHGSASDKTHTCP
PCPAPELLGGPSVFLFPPKPKDTLMISRTPEVTCVVVDVSHEDPEVKFNW
YVDGVEVHNAKTKPREEQYNSTYRVVSVLTVLHQDWLNGKEYKCKVSNKA
LPAPIEKTISKAKGQPREPQVYTLPPSREEMTKNQVSLTCLVKGFYPSDI
AVEWESNGQPENNYKTTPPVLDSDGSFFLYSKLTVDKSRWQQGNVFSCSV
MHEALHNHYTQKSLSLSPGK

### ScFvs as a Therapeutic Domain

An ScFv of trastuzumab was made. Two trastuzumab ScFvs were designed, as follows.

Engineered trastuzumab ScFv 1 (LH)
(SEQ ID NO: 94)
DIQMTQSPSSLSASVGDRVTITCRASQDVNTAVAWYQQKPGKAPKLLIYS
ASFLYSGVPSRFSGSRSGTDFTLTISSLQPEDFATYYCQQHYTTPPTFGQ
GTKVEIKGGGGSGGGGSGGGGSEVQLVESGGGLVQPGGSLRLSCAASGFN
IKDTYIHWVRQAPGKGLEWVARIYPTNGYTRYADSVKGRFTISADTSKNT
AYLQMNSLRAEDTAVYYCSRWGGDGFYAMDYWGQGTLVTVSSGGGSGGGS
LSGRSDNHGGGSHHHHHHHH
Engineered trastuzumab ScFv (HL)
(SEQ ID NO: 95)
EVQLVESGGGLVQPGGSLRLSCAASGFNIKDTYIHWVRQAPGKGLEWVAR
IYPTNGYTRYADSVKGRFTISADTSKNTAYLQMNSLRAEDTAVYYCSRWG
GDGFYAMDYWGQGTLVTVSSGGGGSGGGGSGGGGSDIQMTQSPSSLSASV
GDRVTITCRASQDVNTAVAWYQQKPGKAPKLLIYSASFLYSGVPSRFSGS
RSGTDFTLTISSLQPEDFATYYCQQHYTTPPTFGQGTKVEIKGGGSGGGS
LSGRSDNHGGGSHHHHHHHH

The design was based on the engineered trastuzumab antibody D3. The cleavable domain is shown in bold, and the cleavage site is underlined.

Trastuzumab D18
(SEQ ID NO: 10)
DIQMTQSPSSLSASVGDRVTITCRASQDVNTAVAWYQQKPGKAPKLLIYS
ASFLYSGVPSRFSGSRSGTDFTLTISSLQPEDFATYYCQQHYTTPPTFGQ
GTKVEIKGGGGSGGGGSGGGGSEVQLVESGGGLVQPGGSLRLSCAASGFN
IKDTYIHWVRQAPGKGLEWVARIYPTNGYTRYADSVKGRFTISADTSKNT
AYLQMNSLRAEDTAVYYCSRWGGDGFYAMDYWGQGTLVTVSSGGGSGGGS
LSGRSDNHGSASDKTHTCPPCPAPELLGGPSVFLFPPKPKDTLMISRTPE
VTCVVVDVSHEDPEVKFNWYVDGVEVHNAKTKPREEQYNSTYRVVSVLTV
LHQDWLNGKEYKCKVSNKALPAPIEKTISKAKGQPREPQVYTLPPSREEM
TKNQVSLTCLVKGFYPSDIAVEWESNGQPENNYKTTPPVLDSDGSFFLYS
KLTVDKSRWQQGNVFSCSVMHEALHNHYTQKSLSLSPGK

A control ScFv of trastuzumab was also made. This design contained a longer linker instead of the cleavable domain. A different light chain was designed, as follows:

Engineered trastuzumab ScFv control (LH)
(SEQ ID NO: 96)
DIQMTQSPSSLSASVGDRVTITCRASQDVNTAVAWYQQKPGKAPKLLIYS
ASFLYSGVPSRFSGSRSGTDFTLTISSLQPEDFATYYCQQHYTTPPTFGQ
GTKVEIKGGGGSGGGGSGGGGSEVQLVESGGGLVQPGGSLRLSCAASGFN
IKDTYIHWVRQAPGKGLEWVARIYPTNGYTRYADSVKGRFTISADTSKNT
AYLQMNSLRAEDTAVYYCSRWGGDGFYAMDYWGQGTLVTVSSGGGSGGGS
GGGSHHHHHHHH

This was combined with the heavy chain Engineered trastuzumab ScFv (HL) to create the engineered trastuzumab ScFv control (long linked).

### Transformation

The ScFvs were expressed in BL21(DE3) E. coli cell lines where they were induced with isopropyl β-D-1-thiogalactopyranoside (IPTG) when the cells are in the log growth phase. The cells will grow to express protein at 30° C. for 16 hours. The bacteria were then harvested, and the cells discarded. The ScFvs were purified using nickel affinity chromatography via the HIS-tag and eluted with phosphate-buffered saline (PBS) buffer containing high concentrations of imidazole. The ScFvs were dialysed against PBS to remove traces of imidazole, and the purity assessed by SDS-PAGE. The ScFv-FC constructs were transformed into EXP-CHO cell lines as described above.

### Purification, Buffer Exchange and SDS-PAGE Analysis

All ScFv samples were purified on Nickle columns (Cytiva) and formulated in 1×PBS pH 7.2. PelB leader sequences gave higher yield of ScFvs, and it didn't matter if the ScFv was in the LH or HL orientation. The engineered agents comprising VHH and both varieties of ScFvs gave good expression levels. ScFv-Fc samples were purified in an identical manner to a monoclonal antibody, as described above.

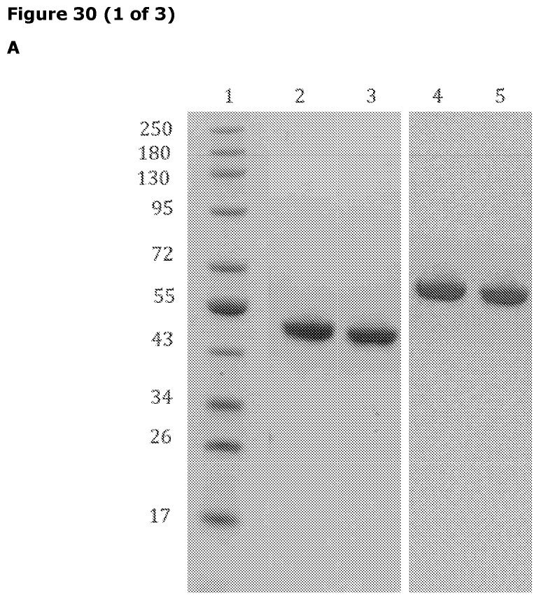

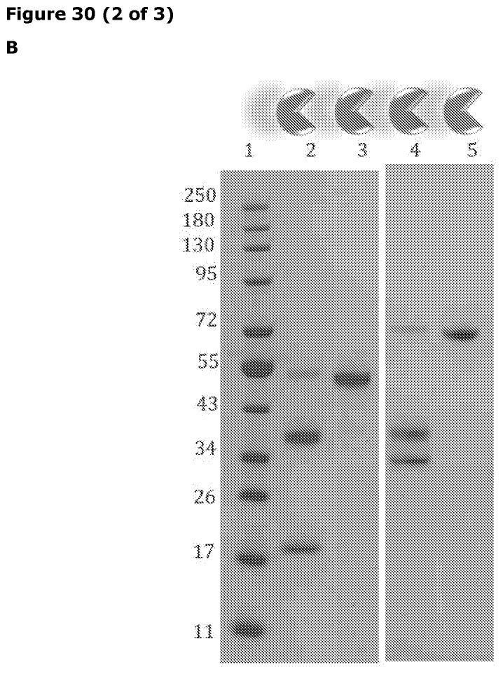

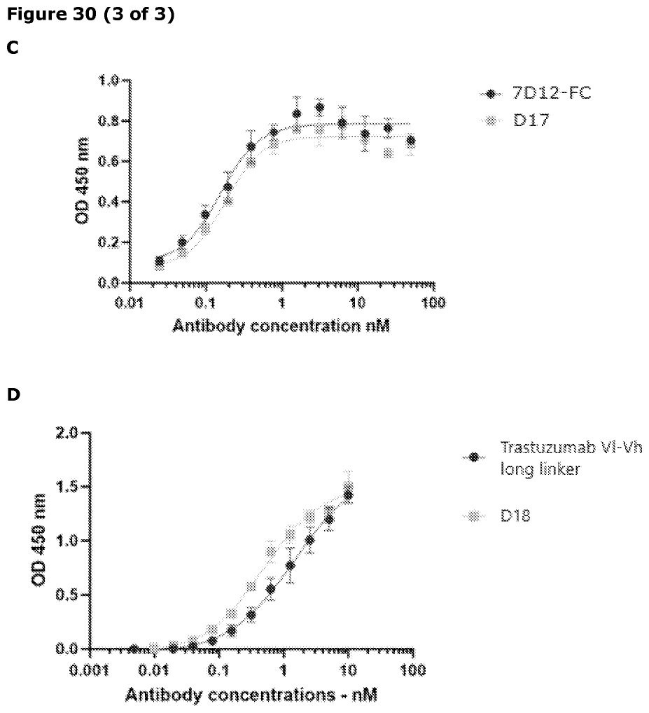

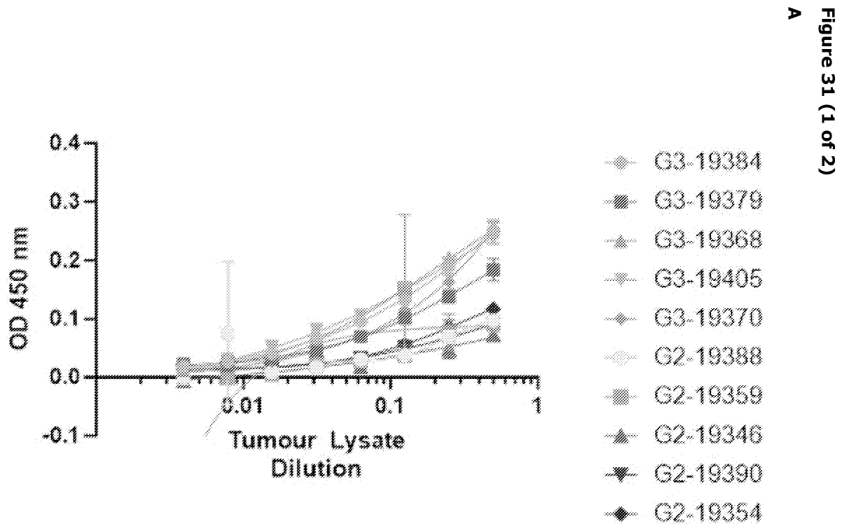

The samples were quality controlled by SDS-PAGE, as described above (see Examples 4 and 6-8). SDS PAGE showed expected expression and purity profiles for all loaded agent (FIG. 30A).

### Protease Digestions of Antibody Variants

The protocol described above (see Example 4 and 6-8) was used to test whether the variants prepared as described above were amenable to digestion.

It was found that uPA cleaved the antibody variants and generated F(ab) fragments in a similar manner as it did for the other designs (FIG. 30B).

Binding of D17 and D18 to their Respective Targets (Assessed by ELISA)

ELISA binding to Her2 was used to determine whether the ScFv retain their binding properties. ELISA binding to hEGFR was used to determine whether the VHH retain their binding properties.

ELISAs were performed as described above in Examples 4, and 6-8, however the antigen used to coat the plate was hHer2-Fc, and after addition of the ScFvs, detection was performed with an anti-HIS-HRP antibody rather than an anti-Vk. Again, the LH or HL orientations in the ScFvs did not matter, and the introduction of the protease site did not affect expression. D18 and D19 were amenable to cleavage and found to retain potency and binding towards hEGFR and hHer2, respectively (as shown in FIGS. 30C, 30D and Table 19).

TABLE 19
IC₅₀ values for controls and engineered agents D7 and D18

| Control | IC₅₀ | Engineered agent | IC₅₀ |
|---|---|---|---|
| 7D12C-FC | 0.1563 | D17 | 0.1819 |
| Trastuzumab Vl-Vh | 1.438 | D18 | 0.4754 |
| long linker | | | |

This example shows that different therapeutic domains can be amenable to this technology.

### Example 9: In Vivo Validation

To gain in vivo validation that protease specific cleavage sites do lead to improved antibody penetration, Trastuzumab and Trastuzumab D3 were expressed at large scale at endo-toxin free grade and quality controlled to ensure they still bind hHer2 at similar levels but that Trastuzumab D3 cuts in the presence of uPA.

A human SVOK-3 ovarian cancer cell line was grown and engrafted into a Balb/C nude immunodeficient mouse in the right upper flank region. 1×10⁷ cells were engrafted in PBS with 1:1 matrigel. Dosing commenced when the tumour size reached 300 mm³, and 5 mg/kg of either Trastuzumab (Group 2 or G2) or Trastuzumab D3 (Group 3 or G3) was added to 5 mice per group (10 mice in total). The study was terminated 24 hours, only a single dose was given. Tumours were harvested, as were serum samples and flash frozen for analysis.

### Binding of Tumour Samples to Human Her2 Using ELISA

Plates were set up as follows: 3 plates of hHer2-HIS diluted to 1 μg/ml, 3 plates of BSA diluted to 1 μg/ml, and 3 plates of PBS blank. 50 μl of each antigen was added to each well and were diluted in PBS. Plates were left to coat overnight at 4° C. The plates were washed, then left for 1 hour at room temperature to block, before being washed again.

10 tumour samples thawed on ice and 150 μl was diluted in 150 μl of PBS+3% dried milk making a 1 in 1 dilution. These were then serially diluted 1 in a 2 dilution over 8 wells. 50 μl was added to each well and the proteins were incubated for 1 hour at room temperature. The plates were prepared with the anti-Vk antibody and samples as described above. The colour was developed, the signal quenched and the plate was read for absorbance at 450 nm.

### Results and Conclusions

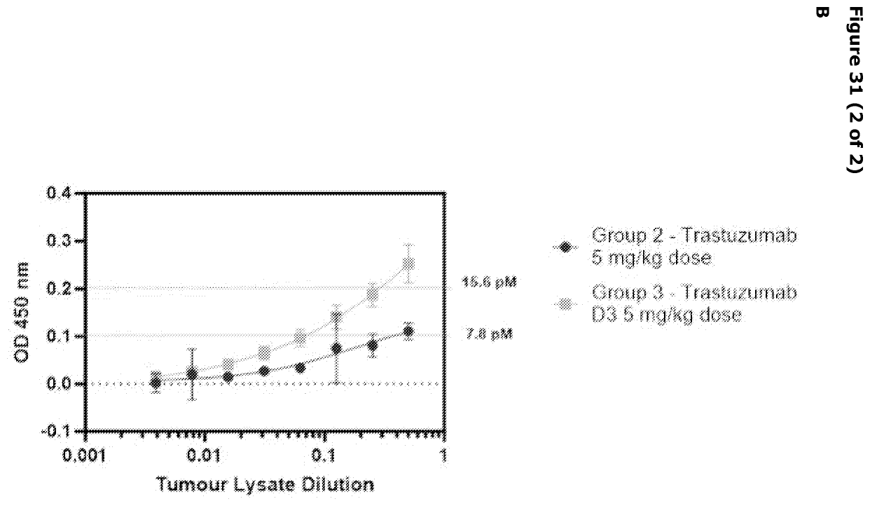

Binding analysis showed that tumours from mice in Group 3 has an increased accumulation of drug in comparison to tumours from mice in Group 2 (see FIG. 31A). Amalgamation of data was performed (see FIG. 31b). Tumours had more than 2-times more engineered agent compared to non-engineered agent, and this difference was significant even after only 24 hours of a single dose of 5 mg/kg.

This example shows that engineering of the agent can improve tumour penetration. It also demonstrates that the levels on endogenous cancer-specific proteases are sufficient for effective cleavage of the engineered agent.

### EXAMPLE EMBODIMENTS

1. An engineered agent comprising:
i. a therapeutic domain;
ii. a cleavable domain; and
iii. a stabilisation domain,
   wherein the cleavable domain is positioned between the therapeutic domain and the stabilising domain.

2. The agent according to Embodiment 1 wherein the agent comprises amino acids.

3. The agent according to any of the preceding embodiments wherein the agent is a protein, peptide, bicyclic peptide, tricyclic peptide or a polypeptide.

4. The agent according to any of the preceding embodiments wherein the agent comprises non-natural isomers or amino acids.

5. The agent according to any of the preceding embodiments wherein the agent has a size which prevents it from penetrating the glomerular filtration barrier.

6. The agent according to any of the preceding embodiments wherein the agent has a size of at least 6 nanometres (nm).

7. The agent according to any of the preceding embodiments wherein the agent has a molecular weight of at least 40 kilodaltons (kDa), at least 50 kDa, at least 70 kDa, at least 100 kDa.

8. The agent according to any one of the preceding embodiments, wherein the half-life of the agent in a biological system is at least 10 hours.

9. The agent according to any of the preceding embodiments wherein the half-life of the agent in a biological system is at least 1.2, 1.4, 1.6, 1.8, 2, 2.2, 2.4, 2.6, 2.8, 3, 4, 5, 6, 7, 8, 9, 10, 11, 12, 13, 14, or at least 15 times longer than the half-life of the therapeutic domain when present in isolation from a) the cleavable domain and the stabilisation domain; or b) the stabilisation domain in said biological system.

10. The agent according to any of the preceding embodiments wherein the therapeutic domain is a domain that, when present in isolation from the cleavable domain and the stabilisation domain, in a biological system, has a half-life of less than 10 hours, less than 9 hours, less than 8 hours, less than 7 hours, less than 6 hours, or less than 5 hours.

11. The agent according to any of the preceding embodiments wherein the ability of the agent to penetrate a solid tumour is decreased relative to the ability of the therapeutic domain when present in isolation from a) the cleavable domain and the stabilisation domain; or b) the stabilisation domain.

12. The agent according to any of the preceding embodiments wherein the ability of the therapeutic domain when present in isolation from a) the cleavable domain and the stabilisation domain; or b) the stabilisation domain, to penetrate a solid tumour is increased relative to the ability of the agent.

13. The agent according to Embodiment 12, wherein the increase is at least a 5-fold increase, at least a 10-fold increase, at least a 15-fold increase, at least a 20-fold increase.

14. The agent according to any of the preceding embodiments wherein the tumour uptake level of the therapeutic domain when present in isolation from a) the cleavable domain and the stabilisation domain; or b) the stabilisation domain is at least 10% ID/g.

15. The agent according to any one of the preceding embodiments wherein the therapeutic domain is selected from any one or more of:
i. an antigen-binding domain;
ii. a Fab region;
iii. a F(ab′)2 region;
iv. a scFv region;
v. a tandem scFv region;
vi. a domain antibody, preferably a single domain antibody (sdAb);
vii. a nanobody;
viii. a monoclonal antibody;
ix. a polyclonal antibody;
x. diabody;
xi. triabody;
xii. tetrabody;
xiii. pentabody;
xiv. hexabody;
xv. an antibody drug conjugate;
xvi. a bispecific peptide, such as a bispecific antibody;
xvii. a multispecific peptide, such as a multispecific antibody;
xviii. a bicyclic peptide; and/or
xix. a tricyclic peptide;
xx. a T-cell receptor (TCR) domain;
xxi. a receptor domain;
xxi. a receptor mimic domain;
xxiii. a cytokine;
xxiv. a hormone;
xxv. a growth factor;
xxvi. a peptide; and/or
xxvii. a derivative of a peptide
   preferably i. an antigen-binding domain;
ii. a Fab region;
iii. a F(ab′)2 region;
iv. a scFv region;
v. a tandem scFv region;
vi. a sdAb.

16. The agent according to any one of the preceding embodiments, wherein the agent is:
i. a monoclonal antibody;
ii. a polyclonal antibody;
iii. diabody;
iv. triabody;
v. tetrabody;
vi. pentabody;
vii. hexabody;
viii. an antibody drug conjugate;
ix. a bispecific peptide, such as a bispecific antibody; and/or
x. a multispecific peptide, such as a multispecific antibody;
preferably i. a monoclonal antibody.

17. The agent according to any one of the preceding embodiments, wherein the therapeutic domain is monovalent, bivalent, trivalent or multivalent.

18. The agent according to any one of the preceding embodiments, wherein the therapeutic domain is a specific binding partner of an entity expressed by a target cell or a target tissue.

19. The agent according to any one of the preceding embodiments, wherein the therapeutic domain is a specific binding partner of an entity expressed by and/or associated with unwanted cells.

20. The agent according to any one of the preceding embodiments, wherein the therapeutic domain is:
i. a T-cell receptor (TCR) domain; or
ii. a receptor domain; or
iii. a receptor mimic domain; or
iv. a cytokine; or
v. a hormone; or
vi. a growth factor; or
vii. a peptide; or
viii. a derivative of a peptide.

21. The agent according to any one of the preceding embodiments, wherein the therapeutic domain binds to an antigen selected from a list comprising Her2, VEGF, PD-1, PMSA, CEA, CTLA-4 or EGFR.

22. The agent according to any one of the preceding embodiments, wherein the cleavable domain does not interfere with the binding of the therapeutic domain to its binding partner.

23. The agent according to any one of the preceding embodiments, wherein the therapeutic domain binds to a binding partner when the cleavable domain has or has not been cleaved.

24. The agent according to any one of the preceding embodiments, wherein the therapeutic domain reduces or inhibits the proliferation of unwanted cells or the growth of a tumour.

25. The agent according to any one of the preceding embodiments, wherein the therapeutic domain stimulates immune cells.

26. The agent according to any one of the preceding embodiments, wherein the therapeutic domain blocks immune cells.

27. The agent according to Embodiments 24-26, wherein the tumour is a solid tumour.

28. The agent according to Embodiments 24-27, wherein the tumour is an in vitro model.

29. The agent according to any one of the preceding embodiments, wherein the stabilisation domain is a protein-based domain or a polymer.

30. The agent according to any one of the preceding embodiments, wherein the stabilisation domain is:
i. a Fc region, optionally wherein the Fc region is an IgG, IgE, IgM, IgD or IgA family Fc region or a bispecific Fc region; or
ii. a PEGylated domain; or
iii. a PASylation domain; or
iv. a XTENylated domain; or
v. a HESylated domain; or
vi. a lipidated domain; or
vii. a glycosylated domain; or
viii. the Human Serum Albumin (HSA) protein or fragment thereof; or
ix. a HSA binding protein; or
x. a protein binding to a blood circulating cell; or
xi. a Fab domain, optionally wherein the Fab domain is directed to HSA or directed to the same antigen as the antigen-binding domain;
preferably i. a Fc region, optionally wherein the Fc region is an IgG, IgE, IgM, IgD or IgA family Fc region or a bispecific Fc region.

31. The agent according to any one of the preceding embodiments, wherein the stabilisation domain displays one or more N-glycosylation motifs.

32. The agent according to any one of the preceding embodiments, wherein the stabilisation domain is selected from the sequences according to SEQ ID Nos: 27-29.

33. The agent according to any one of the preceding embodiments, wherein selective cleavage of the cleavable domain enables the release of the therapeutic domain from the stabilisation domain.

34. The agent according to any one of the preceding embodiments, wherein the cleavable domain comprises at least one cleavage site for an enzyme, optionally wherein the enzyme is a protease.

35. The agent according to Embodiment 34, wherein the protease is a protease that is expressed by and/or accumulates in the vicinity of unwanted cells or a tumour.

36. The agent according to Embodiment 34 or 35 wherein the protease is a tumour-specific protease such as u plasminogen activator (uPA), Napsin A, ADAM10, FAP, matriptase, legumain, MT-SP1, cysteine proteases, serine proteases, metalloproteases, or combination thereof.

37. The agent according to any one of the preceding embodiments, wherein the cleavable domain comprises one or more linker domains present between the therapeutic domain and the stabilisation domain.

38. The agent according to any one of the preceding embodiments wherein the cleavable domain overlaps with the therapeutic domain and/or the stabilisation domain.

39. The agent according to any one of the preceding embodiments, wherein the at least one cleavage site is 4 to 20 amino acids in length.

40. The agent according to of any one of the preceding embodiments wherein the cleavable domain is located in a hinge region, optionally wherein the cleavable domain is located in the upper hinge or in the lower hinge.

41. The agent according to any one of the preceding embodiments, wherein the agent is a protein or polypeptide, the stabilisation domain comprises a Fc region and a hinge region and the cleavable domain is located
i. N-terminal to the hinge region and after the therapeutic domain; or
ii. C-terminal to the hinge region and before the CH2 domain of the Fc region.

42. The agent according to any of Embodiments 1-41 for use as a medicament, such as for use in therapy.

43. The agent according to any of Embodiment 1-41 for use in the treatment or prevention of a tumour, optionally of a cancer.

44. The agent for use according to Embodiment 43 wherein the vicinity of unwanted cells or the tumour comprises a high titre and/or a high activity of tumour-specific proteases.

45. The agent according to Embodiment 43 or 44, wherein the tumour optionally the cancer is in a subject, optionally a human subject.

46. The agent according to any of Embodiment 1-41 for use in a method for the treatment or prevention of a tumour, optionally of cancer, wherein the method comprises:
a) obtaining a sample of the tumour and/or the vicinity of the tumour;
b) determining one or more proteases expressed by the tumour and/or present in the vicinity of the tumour;
c) administering to the subject an agent according to any of Embodiments 1-41 wherein the cleavable domain is cleavable by the one or more proteases determined to be expressed by said tumour and/or present in said vicinity of the tumour.

47. A method of treating or preventing cancer, wherein the method comprises administering one or more agents according to any of Embodiments 1-41.

48. A pharmaceutical composition, comprising an agent according to any of Embodiments 1-41, and optionally a pharmaceutically acceptable carrier, diluent or excipient.

49. The agent according to any of Embodiments 1-41 for use in preventing or treating a condition characterised by the presence of unwanted cells, optionally wherein the condition is cancer.

50. A method of improving the penetrability of a therapeutic domain into a tissue or tumour, wherein the method comprises engineering an agent comprising:
i. a therapeutic domain;
ii. a cleavable domain; and
iii. a stabilisation domain.

51. Use of the agent according to any of Embodiments 1-41 in the manufacture of a medicament for the treatment or prevention of a tumour, optionally for the treatment or prevention of cancer.

52. A method of improving the efficacy of an agent, the method comprising engineering the agent to comprise
i. a therapeutic domain;
ii. a cleavable domain; and
iii. a stabilisation domain,
   wherein the cleavable domain is positioned between the therapeutic domain and the stabilising domain.

53. A method of improving the specificity of an agent, the method comprising engineering the agent to comprise
i. a therapeutic domain;
ii. a cleavable domain; and
iii. a stabilisation domain,
   wherein the cleavable domain is positioned between the therapeutic domain and the stabilising domain.

54. The method according to Embodiment 53, wherein the improved specificity of the agent is manifest by a higher titre of the therapeutic domain in diseased tissue compared to the titre of the therapeutic domain in non-diseased tissue.

55. The method according to any one of Embodiments 50 to 54, wherein the method further comprises:
a) applying the agent according to any of Embodiments 1-41;
b) the cleavable domain being cleaved at the tumour; and
c) the therapeutic domain separating from the stabilisation domain.

56. Use of an agent comprising:
i. a therapeutic domain;
ii. a cleavable domain; and
iii. a stabilisation domain
   to improve the penetrability of the therapeutic domain into a tumour.

57. Use of an agent comprising:
i. therapeutic domain;
ii. a cleavable domain; and
iii. a stabilisation domain
   to reduce the size of the agent at a tumour site.

58. Use of an agent comprising:
i. a therapeutic domain;
ii. a cleavable domain; and
iii. a stabilisation domain,
   wherein the cleavable domain is positioned between the therapeutic domain and the stabilising domain,
   to improve the efficacy of the agent.

59. Use of an agent comprising:
i. a therapeutic domain;
ii. a cleavable domain; and
iii. a stabilisation domain,
   wherein the cleavable domain is positioned between the therapeutic domain and the stabilising domain,
   to improve the specificity of the agent.

60. Use of a cleavable domain to improve the penetrability of a therapeutic domain into a tumour, the use comprising engineering an agent to comprise:
i. a therapeutic domain;
ii. a cleavable domain; and
iii. a stabilisation domain,
   wherein the cleavable domain is positioned between the therapeutic domain and the stabilising domain.

61. Use of a cleavable domain to reduce the size of the agent at a tumour site, the use comprising engineering an agent to comprise:
i. a therapeutic domain;
ii. a cleavable domain; and
iii. a stabilisation domain,
   wherein the cleavable domain is positioned between the therapeutic domain and the stabilising domain.

62. Use of a cleavable domain to improve the efficacy of an agent, the use comprising engineering the agent to comprise:
i. a therapeutic domain;
ii. a cleavable domain; and
iii. a stabilisation domain,
   wherein the cleavable domain is positioned between the therapeutic domain and the stabilising domain.

63. Use of a cleavable domain to improve the specificity of an agent, the use comprising engineering the agent to comprise:
i. a therapeutic domain;
ii. a cleavable domain; and
iii. a stabilisation domain,
   wherein the cleavable domain is positioned between the therapeutic domain and the stabilising domain.

64. The use according to Embodiment 59 or 63, wherein the improved specificity of the agent is manifest by a higher titre of the therapeutic domain in diseased tissue compared to the titre of the therapeutic domain in non-diseased tissue.

65. The use according to Embodiments 57-64, wherein the agent is the agent according to any of Embodiments 1-41.

66. The agent according to any of Embodiments 1-41 for use in diagnosing a disease or disorder.

67. The agent according to any of Embodiments 1-41 for use according to Embodiment 66, wherein the use comprises:
i. administering the agent to a subject;
ii. the cleavable domain being cleaved at the tumour; and
iii. the therapeutic domain separating from the stabilisation domain;
iv. increased accessibility to the cells for binding of the therapeutic domain compared to if cleavage and separation according to steps (ii) and (iii) had not occurred.

68. The agent according to any one of the preceding embodiments, wherein selective cleavage of the cleavable domain enables the release of the therapeutic domain from the stabilisation domain.

69. The agent according to any one of the preceding embodiments, wherein selective cleavage of the cleavable domain enables the release of the therapeutic domain at or near to the cell surface of the unwanted cells.

70. The agent according to any one of the preceding embodiments, wherein the cleaved therapeutic domain has a molecular weight of no more than 50 kDa, no more than 40 kDa, no more than 30 kDa, no more than 25 kDa, no more than 20 kDa, no more than 20 kDa, no more than 15 kDa.

71. The agent according to any one of the preceding embodiments, wherein the agent or the therapeutic domain is a Trastuzumab-derived fragment, a Bevacizumab-derived fragment, a Pembrolizumab-derived fragment, a Cetuximab-derived fragment, a M5A-derived fragment, or an Ipilimumab-derived fragment.

72. The agent according to any one of the preceding embodiments, wherein the therapeutic domain binds its target with a half maximal inhibitory concentration (IC₅₀) of from 0.2 to 1.4 nM, such as from 0.22 to 0.36 nM.

73. The agent according to any one of the preceding embodiments, wherein the therapeutic domain has a dissociation constant (K_{D}) of from 3 to 4.5 nanomolar (nM).

74. The agent according to any one of the preceding embodiments, wherein the cleavable domain does not interfere with the thermal stability of the agent.

75. The agent according to any one of the preceding embodiments, wherein the agent has a melting temperature (Tₘ) of from 65° C. to 75° C., such as from 69° C. to 71° C.

76. The agent according to any one of the preceding embodiments, wherein the agent has an aggregation temperature (T_{agg}) of from 65° C. to 80° C., such as from 66° C. to 76° C.

77. The agent according to any one of the preceding embodiments, wherein the cleavable domain does not interfere with the binding of the stabilisation domain to Fc gamma receptors, optionally wherein the Fc gamma receptors is Fc gamma receptor Ia and/or Fc gamma receptor IIa.

1: An engineered agent comprising:
i. a therapeutic domain;
ii. a cleavable domain; and
iii. a stabilisation domain
wherein the cleavable domain is located in a hinge region and is positioned between the therapeutic domain and the stabilisation domain;
wherein the stabilisation domain is a Fc region;
wherein the cleavable domain comprises a tumour-specific protease cleavage site; and
wherein the therapeutic domain is selected from any one or more of:
i. an antigen-binding domain;
ii. a Fab region;
iii. a F(ab′)2 region;
iv. a scFv region;
v. a tandem scFv region;
vi. a domain antibody;
vii. a nanobody;
viii. a diabody;
ix. a monoclonal antibody;
x. a polyclonal antibody;
xi. triabody;
xii. tetrabody;
xiii. pentabody;
xiv. hexabody;
xv. an antibody drug conjugate;
xvi. a bispecific peptide; and
xvii. a multispecific peptide.

2: The agent according to claim 1 wherein the agent has a size which prevents it from penetrating the glomerular filtration barrier.

3: The agent according to claim 1 wherein the agent has:
i. a size of at least 6 nanometres (nm); and/or
ii. a molecular weight of at least 40 kilodaltons (kDa), at least 50 kDa, at least 70 kDa, at least 100 kDa.

4: The agent according to claim 1, wherein the half-life of the agent in a biological system is:
i. at least 10 hours; and/or
ii. at least 1.2, 1.4, 1.6, 1.8, 2, 2.2, 2.4, 2.6, 2.8, 3, 4, 5, 6, 7, 8, 9, 10, 11, 12, 13, 14, or at least 15 times longer than the half-life of the therapeutic domain when present in isolation from a) the cleavable domain and the stabilisation domain; or b) the stabilisation domain in said biological system.

5: The agent according to claim 1 wherein the therapeutic domain is a domain that, when present in isolation from the cleavable domain and the stabilisation domain, in a biological system, has a half-life of less than 10 hours, less than 9 hours, less than 8 hours, less than 7 hours, less than 6 hours, or less than 5 hours.

6: The agent according to claim 1 wherein the ability of:
i. the agent to penetrate a solid tumour is decreased relative to the ability of the therapeutic domain when present in isolation from a) the cleavable domain and the stabilisation domain; or b) the stabilisation domain; and/or
ii. the therapeutic domain when present in isolation from a) the cleavable domain and the stabilisation domain; or b) the stabilisation domain, to penetrate a solid tumour is increased relative to the ability of the agent, optionally wherein the increase is at least a 5-fold increase, at least a 10-fold increase, at least a 15-fold increase, at least a 20-fold increase.

7: The agent according to claim 1 wherein the tumour uptake level of the therapeutic domain when present in isolation from a) the cleavable domain and the stabilisation domain; or b) the stabilisation domain is at least 10% ID/g.

8: The agent according to claim 1 wherein the domain antibody is a single domain antibody (sdAb); and/or
the bispecific peptide is a bispecific antibody; and/or
the multispecific peptide is a multispecific antibody.

9: The agent according to claim 1, wherein the cleaved therapeutic domain has a molecular weight of no more than 50 kDa, no more than 40 kDa, no more than 30 kDa, no more than 25 kDa, no more than 20 kDa, no more than 20 kDa, no more than 15 kDa.

10: The agent according to claim 1, wherein the agent is:
i. a monoclonal antibody;
ii. a polyclonal antibody;
iii. diabody;
iv. triabody;
v. tetrabody;
vi. pentabody;
vii. hexabody;
viii. an antibody drug conjugate;
ix. a bispecific peptide, such as a bispecific antibody; and/or
x. a multispecific peptide, such as a multispecific antibody,
preferably i. a monoclonal antibody.

11: The agent according to claim 1, wherein the therapeutic domain is a specific binding partner of an entity:
i. expressed by a target cell or a target tissue; and/or
ii. expressed by and/or associated with unwanted cells.

12: The agent according to claim 1, wherein the therapeutic domain binds to an antigen selected from a list comprising Her2, VEGF, PD-1, PMSA, CEA, CTLA-4, EGFR or PDL1; and/or wherein the agent or the therapeutic domain is a Trastuzumab-derived fragment, a Bevacizumab-derived fragment, a Pembrolizumab-derived fragment, a Cetuximab-derived fragment, a M5A-derived fragment, or an Ipilimumab-derived fragment.

13. (canceled)

14: The agent according to claim 1, wherein the cleavable domain does not interfere with the binding of the therapeutic domain to its binding partner.

15. (canceled)

16: The agent according to claim 1, wherein the therapeutic domain binds its target with an IC₅₀ of from 0.2 to 1.4 nM, such as from 0.22 to 0.36 nM and/or has a KD of from 3 to 4.5 nM.

17. (canceled)

18. (canceled)

19: The agent according to claim 1, wherein the Fc region is an IgG, IgE, IgM, IgD or IgA family Fc region or a bispecific Fc region.

20: The agent according to claim 1, wherein the tumour-specific protease site is cleavable by u plasminogen activator (upA), Napsin A, ADAM10, fibroblast activation protein (FAP), matriptase, legumain, MT-SP1, cysteine proteases, serine proteases, metalloproteases, or a combination thereof.

21. (canceled)

22. (canceled)

23: The agent according to claim 1 wherein the cleavable domain is located in the upper hinge or in the lower hinge, preferably wherein the cleavable domain is located in the upper hinge.

24: The agent according to claim 1, wherein where the agent is a protein or polypeptide, the stabilisation domain comprises a Fc region and a hinge region and the cleavable domain is located:
i. N-terminal to the hinge region and after the therapeutic domain; or
ii. C-terminal to the hinge region and before the CH2 domain of the Fc region.

25. (canceled)

26. (canceled)

27: A method of treating or preventing cancer, wherein the method comprises administering one or more agents according to claim 1.

28. (canceled)

29. (canceled)

30: A method of improving the penetrability of a therapeutic domain into a tissue or tumour, wherein the method comprises engineering an agent comprising:
i) a therapeutic domain;
ii) a cleavable domain; and
iii) a stabilisation domain;
wherein the cleavable domain is located in a hinge region and is positioned between the therapeutic domain and the stabilisation domain;
wherein the stabilisation domain is a Fc region;
wherein the cleavable domain comprises a tumour-specific protease cleavage site; and
wherein the therapeutic domain is selected from any one or more of:
i. an antigen-binding domain;
ii. a Fab region;
iii. a F(ab′)2 region;
iv. a scFv region;
v. a tandem scFv region;
vi. a domain antibody;
vii. a nanobody;
viii. a diabody;
ix. a monoclonal antibody;
x. a polyclonal antibody;
xi. triabody;
xii. tetrabody;
xiii. pentabody;
xiv. hexabody;
xv. an antibody drug conjugate;
xvi. a bispecific peptide; and
xvii. a multispecific peptide.

31. (canceled)


SEQUENCE LISTING (96 sequences)

SEQ ID NO: 1 - protein, 460 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGSLSGRSDNHDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 458 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCLSGRSDNHDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 462 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGSLSGRSDNHGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 453 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCLSGRSDNHCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 454 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCLSGRSDNHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 458 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPLSGRSDNHELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 450 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPLSGRSDNHFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 465 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGSPRAEALKGGGSASDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 370 aa, synthetic construct
QVQLQESGGGLVQPGGSLRLSCAASGRTFSSYAMGWFRQAPGKQREFVAAIRWSGGYTYYTDSVKGRFTISRDNAKTTVYLQMNSLKPEDTAVYYCAATYLSSDYSRYALPQRPLDYDYWGQGTQVTVSSSLSGRSDNHGSASDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 489 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGGGGSGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGSGGGSLSGRSDNHGSASDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 463 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGSLSGRSDNHGSASDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 467 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGSLSGRSDNHGSASDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 459 aa, synthetic construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGSLSGRSDNHGSGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 14 - protein, 459 aa, synthetic construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPGSLSGRSDNHGSCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 15 - protein, 465 aa, synthetic construct
EVQLVESGGGLVQPGGSLRLSCAASGFNIKDTYMHWVRQAPGKGLEWVARIDPANGNSKYADSVKGRFTISADTSKNTAYLQMNSLRAEDTAVYYCAPFGYYVSDYAMAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGSLSGRSDNHGSASDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 462 aa, synthetic construct
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCGSLSGRSDNHGSASDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 223 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 18 - protein, 235 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAP

SEQ ID NO: 19 - protein, 131 aa, synthetic construct
QVQLQESGGGLVQPGGSLRLSCAASGRTFSSYAMGWFRQAPGKQREFVAAIRWSGGYTYYTDSVKGRFTISRDNAKTTVYLQMNSLKPEDTAVYYCAATYLSSDYSRYALPQRPLDYDYWGQGTQVTVSSS

SEQ ID NO: 20 - protein, 242 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGGGGSGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSS

SEQ ID NO: 21 - protein, 222 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 22 - protein, 226 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 23 - protein, 222 aa, synthetic construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKY

SEQ ID NO: 24 - protein, 224 aa, synthetic construct
EVQLVESGGGLVQPGGSLRLSCAASGFNIKDTYMHWVRQAPGKGLEWVARIDPANGNSKYADSVKGRFTISADTSKNTAYLQMNSLRAEDTAVYYCAPFGYYVSDYAMAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 25 - protein, 221 aa, synthetic construct
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 26 - protein, 5 aa, synthetic construct
YNSTY

SEQ ID NO: 27 - protein, 215 aa, synthetic construct
ELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - protein, 207 aa, synthetic construct
FLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 29 - protein, 215 aa, synthetic construct
EFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 30 - protein, 22 aa, synthetic construct
GSLSGRSDNHDKTHTCPPCPAP

SEQ ID NO: 31 - protein, 20 aa, synthetic construct
LSGRSDNHDKTHTCPPCPAP

SEQ ID NO: 32 - protein, 24 aa, synthetic construct
GSLSGRSDNHGSDKTHTCPPCPAP

SEQ ID NO: 33 - protein, 15 aa, synthetic construct
LSGRSDNHCPPCPAP

SEQ ID NO: 34 - protein, 16 aa, synthetic construct
LSGRSDNHTCPPCPAP

SEQ ID NO: 35 - protein, 20 aa, synthetic construct
DKTHTCPPCPAPLSGRSDNH

SEQ ID NO: 36 - protein, 27 aa, synthetic construct
GSPRAEALKGGGSASDKTHTCPPCPAP

SEQ ID NO: 37 - protein, 26 aa, synthetic construct
GSLSGRSDNHGSASDKTHTCPPCPAP

SEQ ID NO: 38 - protein, 22 aa, synthetic construct
GSLSGRSDNHGSGPPCPPCPAP

SEQ ID NO: 39 - protein, 22 aa, synthetic construct
GPPGSLSGRSDNHGSCPPCPAP

SEQ ID NO: 40 - protein, 24 aa, synthetic construct
LSGRSDNHGSASDKTHTCPPCPAP

SEQ ID NO: 41 - protein, 32 aa, synthetic construct
GGGSGGGSLSGRSDNHGSASDKTHTCPPCPAP

SEQ ID NO: 42 - protein, 5 aa, unidentified
GGNGT

SEQ ID NO: 43 - protein, 5 aa, synthetic construct
YGNGT

SEQ ID NO: 44 - protein, 4 aa, synthetic construct
GGGS

SEQ ID NO: 45 - protein, 8 aa, synthetic construct
GGGSGGGS

SEQ ID NO: 46 - protein, 5 aa, synthetic construct
GPPGS

SEQ ID NO: 47 - protein, 5 aa, synthetic construct
GSGPP

SEQ ID NO: 48 - protein, 16 aa, synthetic construct
AKTTPKLEEGEFSEAR

SEQ ID NO: 49 - protein, 17 aa, synthetic construct
AKTTPKLEEGEFSEARV

SEQ ID NO: 50 - protein, 9 aa, synthetic construct
AKTTPKLGG

SEQ ID NO: 51 - protein, 10 aa, synthetic construct
SAKTTPKLGG

SEQ ID NO: 52 - protein, 17 aa, synthetic construct
AKTTPKLEEGEFSEARV

SEQ ID NO: 53 - protein, 6 aa, synthetic construct
SAKTTP

SEQ ID NO: 54 - protein, 10 aa, synthetic construct
SAKTTPKLGG

SEQ ID NO: 55 - protein, 6 aa, synthetic construct
RADAAP

SEQ ID NO: 56 - protein, 9 aa, synthetic construct
RADAAPTVS

SEQ ID NO: 57 - protein, 12 aa, synthetic construct
RADAAAAGGPGS

SEQ ID NO: 58 - protein, 27 aa, synthetic construct
RADAAAAGGGGSGGGGSGGGGSGGGGS

SEQ ID NO: 59 - protein, 6 aa, synthetic construct
SAKTTP

SEQ ID NO: 60 - protein, 10 aa, synthetic construct
SAKTTPKLGG

SEQ ID NO: 61 - protein, 18 aa, synthetic construct
SAKTTPKLEEGEFSEARV

SEQ ID NO: 62 - protein, 5 aa, synthetic construct
ADAAP

SEQ ID NO: 63 - protein, 12 aa, synthetic construct
ADAAPTVSIFPP

SEQ ID NO: 64 - protein, 5 aa, synthetic construct
TVAAP

SEQ ID NO: 65 - protein, 12 aa, synthetic construct
TVAAPSVFIFPP

SEQ ID NO: 66 - protein, 6 aa, synthetic construct
QPKAAP

SEQ ID NO: 67 - protein, 13 aa, synthetic construct
QPKAAPSVTLFPP

SEQ ID NO: 68 - protein, 6 aa, synthetic construct
AKTTPP

SEQ ID NO: 69 - protein, 13 aa, synthetic construct
AKTTPPSVTPLAP

SEQ ID NO: 70 - protein, 6 aa, synthetic construct
AKTTAP

SEQ ID NO: 71 - protein, 13 aa, synthetic construct
AKTTAPSVYPLAP

SEQ ID NO: 72 - protein, 6 aa, synthetic construct
ASTKGP

SEQ ID NO: 73 - protein, 13 aa, synthetic construct
ASTKGPSVFPLAP

SEQ ID NO: 74 - protein, 15 aa, synthetic construct
GENKVEYAPALMALS

SEQ ID NO: 75 - protein, 15 aa, synthetic construct
GPAKELTPLKEAKVS

SEQ ID NO: 76 - protein, 15 aa, synthetic construct
GHEAAAVMQVQYPAS

SEQ ID NO: 77 - protein, 8 aa, synthetic construct
LSGRSDNH

SEQ ID NO: 78 - protein, 5 aa, synthetic construct
DKTHT

SEQ ID NO: 79 - protein, 4 aa, synthetic construct
CPPC

SEQ ID NO: 80 - protein, 9 aa, synthetic construct
LSGRSDNHT

SEQ ID NO: 81 - protein, 8 aa, synthetic construct
ELLGGPSV

SEQ ID NO: 82 - DNA, 731 bp, synthetic construct
  source 1..731: mol_type = other DNA
attaagctcttccctggccgaggttcaactggttgagagtggagggggattggtgcagccaggcggcagtctgcggttgagctgcgcagcctctggattcaatatcaaagatacttacatacactgggtccgacaggcacctggcaaggggcttgagtgggttgcccgtatttatcccactaacggttatacacggtacgccgactctgttaaaggtcgattcacaataagtgcagatacctccaaaaacacagcttatctgcaaatgaacagccttcgcgcagaagatactgcagtttattattgctcacgctggggtggggatggcttctacgctatggactattgggggcagggcaccctggtcactgtatcctctgcctctacaaaaggtccatccgtattcccattggctccaagctctaagagtacttctggtggtacagccgcccttggatgtctcgtaaaagattacttccctgaacctgtgaccgtctcctggaattcaggggcactgacaagcggcgttcatacttttcctgccgttcttcagtcaagtggcctttacagtctgagttccgtcgtaacagtccctagctcaagtcttgggacacagacttatatctgcaacgtaaatcacaaaccctctaatactaaggtagacaagaaagtggagcccaaatcctgtgggtctctctccggtcgatccgacaaccacgctggaagagcgg

SEQ ID NO: 83 - DNA, 725 bp, synthetic construct
  source 1..725: mol_type = other DNA
attaagctcttccctggccgaggttcaactggttgagagtggagggggattggtgcagccaggcggcagtctgcggttgagctgcgcagcctctggattcaatatcaaagatacttacatacactgggtccgacaggcacctggcaaggggcttgagtgggttgcccgtatttatcccactaacggttatacacggtacgccgactctgttaaaggtcgattcacaataagtgcagatacctccaaaaacacagcttatctgcaaatgaacagccttcgcgcagaagatactgcagtttattattgctcacgctggggtggggatggcttctacgctatggactattgggggcagggcaccctggtcactgtatcctctgcctctacaaaaggtccatccgtattcccattggctccaagctctaagagtacttctggtggtacagccgcccttggatgtctcgtaaaagattacttccctgaacctgtgaccgtctcctggaattcaggggcactgacaagcggcgttcatacttttcctgccgttcttcagtcaagtggcctttacagtctgagttccgtcgtaacagtccctagctcaagtcttgggacacagacttatatctgcaacgtaaatcacaaaccctctaatactaaggtagacaagaaagtggagcccaaatcctgtctctccggtcgatccgacaaccacgctggaagagcgg

SEQ ID NO: 84 - DNA, 737 bp, synthetic construct
  source 1..737: mol_type = other DNA
attaagctcttccctggccgaggttcaactggttgagagtggagggggattggtgcagccaggcggcagtctgcggttgagctgcgcagcctctggattcaatatcaaagatacttacatacactgggtccgacaggcacctggcaaggggcttgagtgggttgcccgtatttatcccactaacggttatacacggtacgccgactctgttaaaggtcgattcacaataagtgcagatacctccaaaaacacagcttatctgcaaatgaacagccttcgcgcagaagatactgcagtttattattgctcacgctggggtggggatggcttctacgctatggactattgggggcagggcaccctggtcactgtatcctctgcctctacaaaaggtccatccgtattcccattggctccaagctctaagagtacttctggtggtacagccgcccttggatgtctcgtaaaagattacttccctgaacctgtgaccgtctcctggaattcaggggcactgacaagcggcgttcatacttttcctgccgttcttcagtcaagtggcctttacagtctgagttccgtcgtaacagtccctagctcaagtcttgggacacagacttatatctgcaacgtaaatcacaaaccctctaatactaaggtagacaagaaagtggagcccaaatcctgtgggtctctctccggtcgatccgacaaccacgggtctgctggaagagcgg

SEQ ID NO: 85 - DNA, 663 bp, synthetic construct
  source 1..663: mol_type = other DNA
atgccattgtaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtctctccggtcgatccgacaaccactgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtattcgcaatc

SEQ ID NO: 86 - DNA, 668 bp, synthetic construct
  source 1..668: mol_type = other DNA
atgccattgtaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtctctccggtcgatccgacaaccacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtattcgcaatctc

SEQ ID NO: 87 - DNA, 678 bp, synthetic construct
  source 1..678: mol_type = other DNA
atgccattgtaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcactctccggtcgatccgacaaccaccctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtattcgcaatc

SEQ ID NO: 88 - DNA, 651 bp, synthetic construct
  source 1..651: mol_type = other DNA
atgccattgtaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcactctccggtcgatccgacaaccacttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtattcgcaatc

SEQ ID NO: 89 - protein, 9 aa, synthetic construct
PRAEALKGG

SEQ ID NO: 90 - protein, 15 aa, synthetic construct
GSPRAEALKGGGSAS

SEQ ID NO: 91 - DNA, 737 bp, synthetic construct
  source 1..737: mol_type = other DNA
attaagctcttccctggccgaggttcaactggttgagagtggagggggattggtgcagccaggcggcagtctgcggttgagctgcgcagcctctggattcaatatcaaagatacttacatacactgggtccgacaggcacctggcaaggggcttgagtgggttgcccgtatttatcccactaacggttatacacggtacgccgactctgttaaaggtcgattcacaataagtgcagatacctccaaaaacacagcttatctgcaaatgaacagccttcgcgcagaagatactgcagtttattattgctcacgctggggtggggatggcttctacgctatggactattgggggcagggcaccctggtcactgtatcctctgcctctacaaaaggtccatccgtattcccattggctccaagctctaagagtacttctggtggtacagccgcccttggatgtctcgtaaaagattacttccctgaacctgtgaccgtctcctggaattcaggggcactgacaagcggcgttcatacttttcctgccgttcttcagtcaagtggcctttacagtctgagttccgtcgtaacagtccctagctcaagtcttgggacacagacttatatctgcaacgtaaatcacaaaccctctaatactaaggtagacaagaaagtggagcccaaatcctgtgggtctccacgaggctgggatgctcatactgggtctgctggaagagcgg

SEQ ID NO: 92 - protein, 476 aa, synthetic construct
QVRLVQSGPQIKTPGASVTISCGTSGYDFMESLINWVRQDIGKGPEWMGWINPRGGGVNYGRRFQGKVTMTRDVSSGTAYLTLRGLTSDDTAKYYCVRGKSCCGGRRYCNGADCFNWDFEHWGQGTLVIVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGSLSGRSDNHGSASDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 93 - DNA, 455 bp, synthetic construct
  source 1..455: mol_type = other DNA
attaagctcttccctggcccaagtccaactgcaggagagcgggggtggcctggtacagccaggaggtagccttcggctgtcatgtgcagccagcggacgcacattttccagttacgcaatgggttggtttaggcaagcccctggtaaacagagagaattcgtcgctgccattcggtggagtggaggttatacatactacacagatagtgttaagggtcgttttactatttctcgggacaatgctaaaaccacagtttatctccagatgaatagtttgaagcccgaggacacagccgtatattattgtgccgccacttatctttctagcgactactcccgttacgccttgccacaacgtcctcttgactatgactattggggacaaggtactcaagtaaccgtttcctcatctctctccggtcgatccgacaaccacgggtctgctggaagagcgg

SEQ ID NO: 94 - protein, 270 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGGGGSGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGSGGGSLSGRSDNHGGGSHHHHHHHH

SEQ ID NO: 95 - protein, 270 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGGSGGGGSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGGGSGGGSLSGRSDNHGGGSHHHHHHHH

SEQ ID NO: 96 - protein, 262 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGGGGSGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGSGGGSGGGSHHHHHHHH